(19) United States
(12) Patent Application Publication
(10) Pub. No.: US 2007/0207093 A1
(43) Pub. Date: Sep. 6, 2007

(54) Hydrazone derivatives and uses thereof

(75) Inventors: Robert W. Bryant, Princeton, NJ (US); Roy Kyle Palmer, Cranbury, NJ (US); Rok Cerne, Plainsboro, NJ (US); Karnail S. Atwal, Pennington, NJ (US); Seunghun Paul Lee, Newtown, PA (US); Anita B. Atwal, Pennington, NJ (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Linquagen Corp.

(21) Appl. No.: 11/592,228

(22) Filed: Nov. 3, 2006

Related U.S. Application Data
(60) Provisional application No. 60/732,634, filed on Nov. 3, 2005.

(51) Int. Cl. A61K 8/49; A61K 8/40

(52) U.S. Cl. 424/49; 514/313; 514/408; 514/357; 514/419; 514/367; 514/394; 514/418; 514/639; 514/237.5

(57) ABSTRACT

The present invention is directed to the use of a compound having the formula 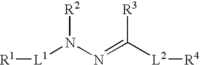
wherein R¹, R², R³, R⁴, L¹, and L² are defined herein. The compounds of the present invention are useful as inhibitors of certain taste perceptions and functions. The invention is also directed to compositions comprising a compound according to the above formula.

The application claims the benefit of U.S. Provisional Application No. 60/732,634, filed Nov. 3, 2005, which is herein incorporated by reference in its entirety.

### BACKGROUND OF THE INVENTION

1. Field of the Invention

The present invention relates to the use of compounds of Formula I for inhibiting certain taste functions and perceptions and related uses. The invention is also directed to, among other things, compositions comprising a compound of Formula I that can be used in pharmaceutical, food, and other products to inhibit certain taste functions and perceptions.

2. Background Art

Taste perception plays a critical role in both the nutritional status of human beings and the basic survival of animals. Margolskee, R. F., J. Biol. Chem. 277:1-4 (2002); Avenet, P. and Lindemann, B., J Membrane Biol. 112:1-8 (1989). The task of taste perception is carried out by taste receptor cells (TRCs). TRCs have the ability to perceive the multitude of compounds that are associated with a given taste and then convert that perception to a signal that is deciphered by the brain, resulting in the sensation of sweet, bitter, sour, salty, or umami (savory) taste.

TRCs are polarized epithelial cells, meaning that they have specialized apical and basolateral membranes. A taste bud contains approximately 60 to 100 TRCs. Each TRC has a portion of its membrane exposed on the mucosal surface of the tongue. Kinnamon, S. C., TINS 11:491-496 (1988). Sensory transduction is initiated by sapid molecules, or “tastants,” that interact with microvillar processes on the apical membrane of TRCs. The tastants bind specific membrane receptors, resulting in a voltage change across the cell membrane. In turn, this depolarizes, or changes the electric potential, of the cell, causing transmitter release and excitation of primary gustatory nerve fibers.

One recently discovered transmembrane protein, TRPM5, has been shown to be essential for taste transduction. Perez et al., Nature Neuroscience 5:1169-1176 (2002); Zhang et al., Cell 112:293-301 (2003). This protein is a member of the transient receptor potential (TRP) family of ion channels, forms a channel through the membrane of the taste receptor cell, and is believed to be activated by stimulation of a receptor pathway coupled to phospholipase C and by IP₃-mediated Ca²⁺ release. The opening of this channel is dependent on a rise in Ca²⁺ levels. Hofmann et al., Current Biol. 13:1153-1158 (2003). The activation of this channel leads to depolarization of the TRC, which in turn leads to transmitter release and excitation of primary gustatory nerve fibers.

Because TRPM5 is a necessary part of the taste-perception machinery, its inhibition prevents an animal from sensing particular tastes. Although taste perception is a vital function, the inhibition of undesirable tastes is beneficial under certain circumstances. For example, many active pharmaceutical ingredients of medicines produce undesirable tastes, such as a bitter taste. Inhibition of the bitter taste produced by the medicine may lead to improved acceptance by the patient.

Traditionally, sweeteners and flavorants have been used to mask the bitter taste of pharmaceuticals. The sweetener or flavorant is known to activate other taste pathways and at sufficiently high concentration this serves to mask the bitter taste of the pharmaceutical. However, this approach has proved ineffective at masking the taste of very bitter compounds. Microencapsulation in a cellulose derivative has also been used to mask the bitter taste of pharmaceuticals. However, this approach prevents rapid oral absorption of the pharmaceutical.

A number of other methods have been suggested to inhibit, alter, or mask unwanted tastes, including the use of 5′-adenosine carboxylic acid (AMP) and 5′-inosine carboxylic acid (IMP) as potential bitterness inhibitors. See U.S. Pat. No. 6,540,978. However, the presently available compounds are lacking in desirable characteristics.

Another aspect of taste is its role in food intake. Studies have shown increased food intake as palatability increased. Sorensen, et al., Int. J. Obes. Relat. Metab. Disord. 27(10):1152-66 (2003). For instance, certain drugs, such as antihypertensives and antihyperlipidemics, have been reported to produce untoward alterations in taste and may result in decreased food intake. Doty, et al., J Hypertens. 21(10):1805-13 (2003). Taste impairment has also been associated with radiation treatments for head and neck cancer and this taste impairment has been considered to be one of the factors associated with reduces appetite and altered patterns of food intake. Vissink, et al., Crit. Rev. Oral Biol. Med. 14(3):213-25 (2003). Decreased food consumption has also been correlated with loss of taste sensations in the elderly. Shiffman, S. S., J. Am. Med. Ass'n 278(16):1357-1362 (1997).

At present, while there are a number of agents that are or have been on the market to reduce appetite and food intake, such as amphetamine derivatives and fenfluramine, many have serious side effects. More selective approaches, e.g., neuro-regulation via peptide mimetics/antagonists, are still in developmental phases.

Therefore, there exists a need for compounds that can effectively inhibit an unwanted taste without exhibiting one or more of the side effects of the prior art taste masking agents.

### SUMMARY OF THE INVENTION

A first aspect of the present invention is directed to a method of inhibiting a taste modulating protein, said method comprising contacting said protein with a compound of Formula I or a physiologically acceptable salt thereof.

An additional aspect of the present invention is directed to a method of inhibiting the depolarization of a taste receptor cell, said method comprising contacting said cell with a compound of Formula I or a physiologically acceptable salt thereof.

An additional aspect of the present invention is directed to a method of inhibiting the taste of a pharmaceutical, comprising administering one or more compounds of Formula I, or a physiologically acceptable salt thereof, in conjunction with the administration of said pharmaceutical to a subject.

An additional aspect of the present invention is directed to a method of inhibiting the taste of a food product, comprising administering one or more compounds of Formula I, or a physiologically acceptable salt thereof, in conjunction with the administration of said pharmaceutical to a subject.

An additional aspect of the present invention is directed to a pharmaceutical composition comprising an active agent, optionally one or more pharmaceutically acceptable carriers, and one or more compounds of Formula I or a physiologically acceptable salt thereof.

An additional aspect of the present invention is directed to a food product comprising one or more compounds according to Formula I or a physiologically acceptable salt thereof.

An additional aspect of the present invention is directed to a method of decreasing the palatability of food and its intake comprising administering one or more compounds of Formula I to a subject in need of such treatment.

These and additional aspects of the present invention are described in detail below.

### BRIEF DESCRIPTION OF THE DRAWINGS/FIGURES

The accompanying drawings, which are incorporated herein and form a part of the specification, serve to explain the principles of the invention and to enable a person skilled in the pertinent art to make and use the invention.

FIG. 1 illustrates the generation of the TRPM5 FLIPR response.

FIG. 2 illustrates eletrophysiology results of inhibiting TRPM5 with the compound of Example 3, as described in Example 24.

FIG. 3 illustrates a summary of 14 experiments demonstrating the inhibition of TRPM5 Ca²⁺ activated current by the compound of Example 3.

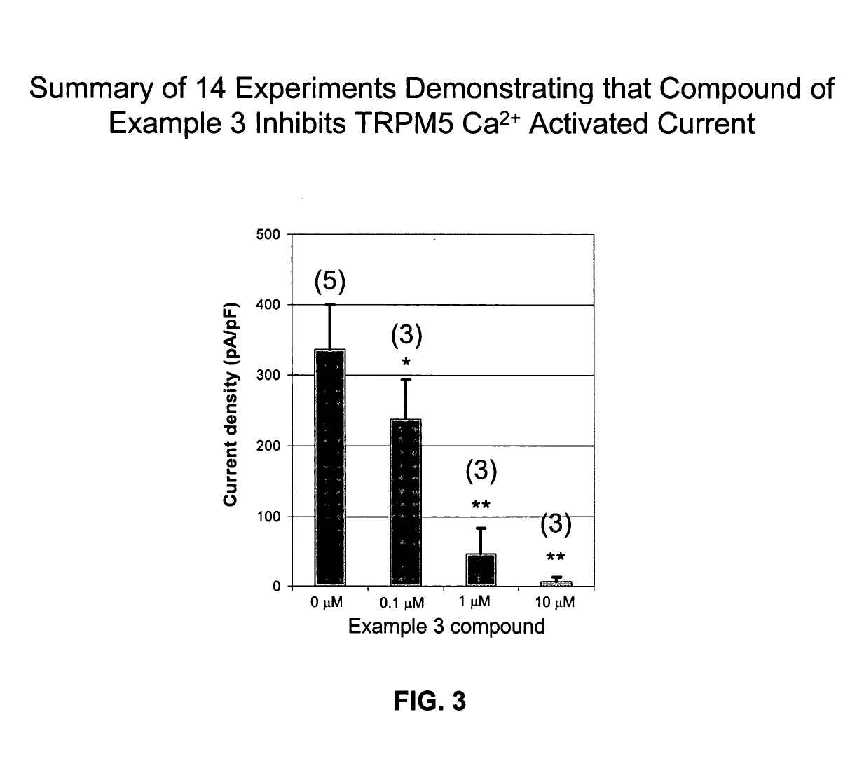

FIGS. 4A and 4B illustrate the TRPM5-dependent fluorescent signal in HEK293 cells, as explained in Example 67.

### DETAILED DESCRIPTION OF THE INVENTION

The present invention provides compounds and compositions that are useful, for example, for inhibiting the activity of a taste modulating protein. Other aspects of the present invention are described in detail herein.

Methods of Use

A first aspect of the present invention is directed to a method of inhibiting a taste modulating protein, said method comprising contacting said protein with a compound of Formula I: 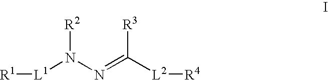
or a physiologically acceptable salt thereof, wherein

R¹ is C₆₋₁₄ aryl, 5-14 membered heteroaryl, C₃₋₁₄ cycloalkyl, C₃₋₁₄ cycloalkenyl, 3-14 membered cycloheteroalkyl, 3-14 membered cycloheteroalkenyl, and C₁₋₆ alkyl, each of which is optionally substituted;

R² is H, C₁₋₆ alkyl, C₆₋₁₀ aryl, or C₆₋₁₀ aryl(C₁₋₆)alkyl;

R³ is H, C₁₋₆ alkyl, C₆₋₁₀ aryl, or cyano;

R⁴ is C₁₋₆ alkyl, C₆₋₁₄ aryl, 5-14 membered heteroaryl, C₃₋₁₄ cycloalkyl, C₃₋₁₄ cycloalkenyl, 3-14 membered cycloheteroalkyl, or 3-14 membered cycloheteroalkenyl, each of which is optionally substituted, or is cyano;

L¹ is absent, or is a linker containing 1-10 carbon and/or heteroatoms and which is optionally substituted;

L² is absent, or is a linker containing 1-10 carbon and/or heteroatoms and which is optionally substituted; or

R³, R⁴, and L², together with the carbon atom to which L² and R³ are attached, form a group selected from C₆₋₁₄ aryl, 5-14 membered heteroaryl, C₃₋₁₄ cycloalkyl, C₃₋₁₄ cycloalkenyl, 3-14 membered cycloheteroalkyl, 3-14 membered cycloheteroalkenyl, each of which is optionally substituted.

In one embodiment, R¹ is optionally substituted C₆₋₁₀ aryl, such as phenyl or naphthyl. In another embodiment, R¹ is optionally substituted 5-10 membered, or preferably 5-7 membered, heteroaryl, such as but not limited to pyridyl, pyrimidinyl, imidazolyl, tetrazolyl, furanyl, thienyl, indolyl, azaindolyl, quinolinyl, pyrrolyl, benzimidazolyl, and benzothiazolyl, each of which is optionally substituted. In other instances, the heteroaryl group is a nitrogen containing heteroaryl or an oxygen containing heteroaryl.

Another subset of R¹ includes a substituted aryl, preferably C₆₋₁₀ aryl, or heteroaryl group having 1-3 substituents independently selected from the group consisting of amino, hydroxy, nitro, halogen, cyano, thiol, C₁₋₆ alkyl, C₂₋₆ alkenyl, C₁₋₆ haloalkyl, C₁₋₆ alkoxy, C₃₋₆ alkenyloxy, C₁₋₆ alkylenedioxy, C₁₋₆ alkoxy(C₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, C₂₋₆ carboxyalkoxy, and C₂₋₆ carboxyalkyl. Another preferred heteroaryl group is carbazolyl, which is optionally substituted.

In another embodiment, R¹ is optionally substituted C₃₋₁₀ cycloalkyl, or optionally substituted C₃₋₁₀ cycloalkenyl. In another embodiment, R¹ is optionally substituted 3-10 membered cycloheteroalkyl or optionally substituted 3-10 membered cycloheteroalkenyl. Suitable R¹ groups include, but are not limited to, cyclopropyl, cyclopentyl, cyclohexyl, cyclopentenyl, cyclohexenyl, and the like. Cycloalkyl groups also include bicycloalkyl and polycycloalkyl groups, preferably having 7-10 carbon atoms, such as bicyclo[4.1.0]heptanyl and adamantyl.

Another subset of R¹ includes a substituted C₃₋₁₀ cycloalkyl or C₃₋₁₀ cycloalkenyl having 1-3 substituents independently selected from the group consisting of amino, hydroxy, nitro, halogen, cyano, thiol, C₁₋₆ alkyl, C₂₋₆ alkenyl, C₁₋₆ haloalkyl, C₁₋₆ alkoxy, C₃₋₆ alkenyloxy, C₁₋₆ alkylenedioxy, C₁₋₆ alkoxy(C₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, C₂₋₆ carboxyalkoxy, and C₂₋₆ carboxyalkyl, each of which is optionally substituted.

In yet a further embodiment, R¹ is optionally substituted C₁₋₆ alkyl, such as methyl, ethyl and propyl. R¹ may be a straight-chain or branched alkyl group. Suitable substituted alkyls include haloalkyl, hydroxyalkyl, aminoalkyl, and the like.

Suitable groups for R¹ include 2-benzo[d]thiazol-2-yl, 1-naphthalenyl, 4-methoxyphenyl, 2-carboxyphenyl, 3-methylphenyl, 3-bromobenzyl, bicyclo[4.1.0]heptanyl, 4-nitrophenyl, 4-(trifluoromethylthio)phenyl, tricyclo[3.3.1.1^{3,7}]decanyl, N-ethyl-N-2-hydroxyethylaminophenyl, 5-Chloro-3-(trifluoromethyl)pyridini-2-yl, 3,4-dimethylphenyl, 2-nitro-5-(pyrrolidin-1-yl)phenyl, 3-cyclohexenyl, and 1H-benzo[d]imidazol-2-yl.

Other suitable groups for R¹ include 4-(dimethylamino)phenyl, 4-(diethylamino)phenyl, 1-hydroxycyclopentyl, 4-nitrophenyl, 2-bromo-4-methoxyphenyl, 1H-indol-3-yl, 4-t-butyl-2-methylphenyl, 4-chlorophenyl, 3-chlorophenyl, 2-chlorophenyl, 4-fluorophenyl, 3-fluorophenyl, 2-fluorophenyl, 8-dimethylquinolin-2-yl, and 9H-carbazol-9-yl.

In another embodiment, R² is H. Alternatively, R² is C₁₋₆ alkyl, such as methyl, ethyl, or propyl. R² may be a straight-chain or branched alkyl group. In other embodiments, R² is a C₆₋₁₀ aryl(C₁₋₆)alkyl, such as benzyl, phenethyl, or phenylpropyl groups. Preferably, R² is a C₆₋₁₀ aryl(C₁₋₄)alkyl.

In a further embodiment, R³ is H. Alternatively, R³ is C₁₋₆ alkyl, such as methyl, ethyl, or propyl. R³ may be a straight-chain or branched alkyl group. In yet another embodiment, R³ is cyano (—CN).

In another embodiment, R⁴ is optionally substituted C₆₋₁₀ aryl, such as phenyl or naphthyl. In another embodiment, R⁴ is optionally substituted 5-10 membered, or preferably 5-7 membered, heteroaryl, such as but not limited to pyridyl, pyrimidinyl, imidazolyl, tetrazolyl, furanyl, thienyl, indolyl, azaindolyl, quinolinyl, pyrrolyl, benzimidazolyl, and benzothiazolyl, each of which is optionally substituted. In other instances, the heteroaryl group is a nitrogen containing heteroaryl. In other instances, the heteroaryl group is an oxygen containing heteroaryl. Another preferred heteroaryl group is carbazolyl, which is optionally substituted.

Another subset of R⁴ includes a substituted aryl or heteroaryl group having 1-3 substituents independently selected from the group consisting of amino, hydroxy, nitro, halogen, cyano, thiol, C₁₋₆ alkyl, C₂₋₆ alkenyl, C₁₋₆ haloalkyl, C₁₋₆ alkoxy, C₃₋₆ alkenyloxy, C₁₋₆ alkylenedioxy, C₁₋₆ alkoxy(C₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, C₂₋₆ carboxyalkoxy, and C₂₋₆ carboxyalkyl.

In another embodiment, R⁴ is optionally substituted C₃₋₁₀ cycloalkyl, or optionally substituted C₃₋₁₀ cycloalkenyl. In another embodiment, R⁴ is optionally substituted 3-10 membered cycloheteroalkyl or optionally substituted 3-10 membered cycloheteroalkenyl. Suitable R⁴ groups include, but are not limited to, cyclopropyl, cyclopentyl, cyclohexyl, cyclopentenyl, cyclohexenyl, and the like. Cycloalkyl groups also include bicycloalkyl groups, such as bicyclo[4.1.0]heptanyl.

In yet a further embodiment, R⁴ is optionally substituted C₁₋₆ alkyl, such as methyl, ethyl, and propyl. R⁴ may be a straight-chain or branched alkyl group. Suitable substituted alkyls include haloalkyl, hydroxyalkyl, aminoalkyl, and the like.

In another embodiment, R4 is a phenyl substituted with 1-4 groups independently selected from the groups consisting of halo, C1-4 alkoxy such as methoxy, and C₁₋₄ alkylthio.

Other suitable R⁴ groups include 6-bromobenzo[d][1,3]dioxol-5-yl, 4-hydroxy-3-iodo-5-methoxybenzylidene, 4-hydroxy-3-methoxyphenyl, 3,4,5-trimethoxyphenyl, 4-(diethylamino)-2-hydroxyphenyl, 5-bromo-2-oxoindolin-3-ylidene, 2-oxoindolin-3-ylidene, 3,4-dimethoxyphenyl, and 3-trifluoromethylphenyl.

Additional suitable groups for R⁴ include 4-methoxyphenyl, 4-(allyloxy)-3-methoxyphenyl, 4-isopropylphenyl, 1,3,3,-indolinylidene, 4-(diethylamino)-2-hydroxyphenyl, 1,5-dimethyl-2-oxoindolin-3-ylidene, 1-butyl-1H-indol-3-yl, 4-pyridinyl, 1H-pyrrol-2-yl, 2,4-dihydroxyphenyl, 4-(4-morpholino)-3-nitrophenyl, quinuclidinylidene, and 2-hydroxy-4-diethylaminophenyl.

In one embodiment, L¹ is absent. Thus, according to this embodiment, R¹ is bonded directly to the nitrogen atom by a single bond.

In another embodiment, L¹ is a linker containing 1-10, preferably 1-7, carbon and/or heteroatoms and which is optionally substituted. The linker is a divalent moiety that connects R¹ to the nitrogen. The linker can be any suitable divalent moiety that contains 1-10 carbon and/or heteroatoms. Suitable linkers will contain, for example, 1, 2, 3, 4, 5, or 6 carbon and/or heteroatoms.

For example, the linker can be a divalent carbon linker with 1-10, preferably 1-7, carbon atoms, such as but not limited to, methylene (—CH₂—), ethylene (—CH₂—CH₂—), propylene (e.g., —CH₂—CH₂—CH₂—), butylene, and the like. Alternatively, L¹ can be a C₃₋₁₀ cycloalkylene linker, such as methylenecyclopropylene. A divalent carbon linker can be substituted with suitable substituents as described herein. In another subset, a preferred group of substituents includes amino, hydroxy, halogen, cyano, thiol, oxo, C₁₋₆ alkyl, C₂₋₆ alkenyl, C₁₋₆ haloalkyl, C₁₋₆ alkoxy, C₃₋₆ alkenyloxy, C₁₋₆ alkoxy(C₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, carboxy, aminocarbonyl, and C₂₋₆ carboxyalkyl.

L¹ can also be a divalent linker that contains 2-10, preferably 2-6, carbon and heteroatoms. Such linkers include, by way of nonlimiting examples, alkyleneoxy, alkyleneamino, alkylenethio, alkylenedioxy. Other suitable examples include —CH₂CH₂C(O)—, —OCH₂—, —NHCH₂—, —OCH₂CH₂—, —NHCH₂CH₂—, and —OCH₂CH₂CH₂—. It is understood that a preferred linker containing both carbon and heteroatoms will be one in which a heteroatom is not directly attached to the nitrogen atom of Formula I.

The linker L¹ can also be contain 1-10 heteroatoms, preferably 1, 2, or 3 heteroatoms. Suitable heteroatom linkers include —O—, —S—, —NH—, —N═N—, and the like. For example, a suitable L¹ group is —SCH₂C(O)—.

In other embodiments, the linker L¹ is a 1-6 membered alkylene, alkenylene, or alkynylene moiety. In other embodiments, the linker L¹ is a 1-6 membered heteroalkylene, heteroalkenylene, or heteroalkynylene moiety.

The linker L¹ can be substituted as described herein. In one embodiment, linker L¹ is a divalent moiety containing 1-6 carbon atoms and substituted with 1, 2, or 3 substituents selected from the group consisting of amino, hydroxy, nitro, halogen, cyano, thiol, oxo, C₁₋₆ alkyl, C₂₋₆ alkenyl, C₁₋₆ haloalkyl, C₁₋₆ alkoxy, C₃₋₆ alkenyloxy, C₁₋₆ alkylenedioxy, C₁₋₆ alkoxy(C₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, C₂₋₆ carboxyalkoxy, benzamido, and C₂₋₆ carboxyalkyl.

In another embodiment, L¹ is a linker selected from the group consisting of

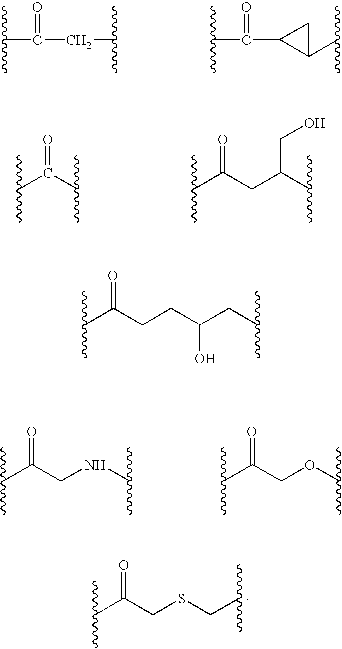

In a further embodiment, R¹ and L¹ together form a group selected from

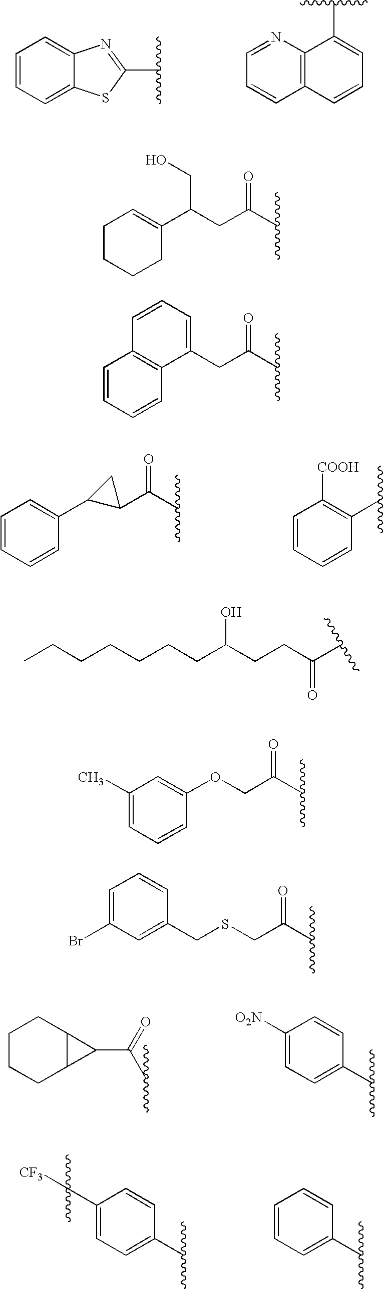

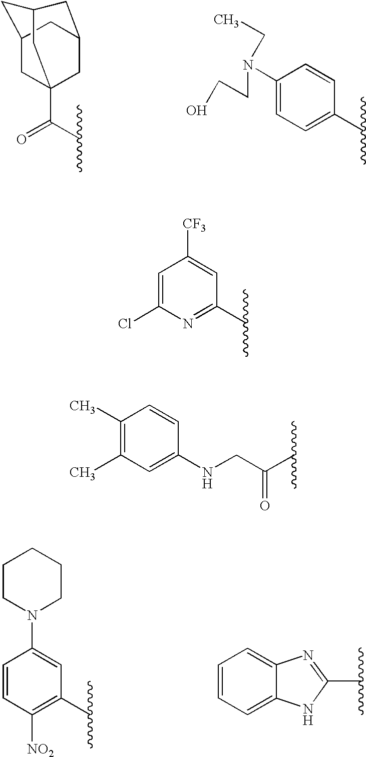

In another embodiment, R¹ and L¹ together form a group selected from the following:

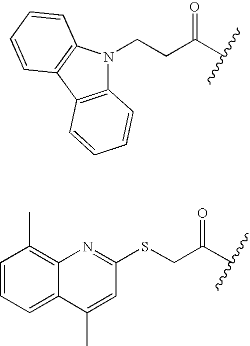

In one embodiment, L² is absent. Thus, according to this embodiment, R⁴ is bonded directly to the carbon atom which is bonded to the nitrogen atom by a double bond.

L² can also be a divalent linker that contains 2-10, preferably 2-6, carbon and heteroatoms. Such linkers include, by way of nonlimiting examples, alkyleneoxy, alkyleneamino, alkylenethio, alkylenedioxy. Other suitable examples include —CH₂CH₂C(O)—, —OCH₂—, —NHCH₂—, —OCH₂CH₂—, —NHCH₂CH₂—, and —OCH₂CH₂CH₂—. It is understood that a preferred linker containing both carbon and heteroatoms will be one in which a heteroatom is not directly attached to the nitrogen atom of Formula I.

The linker L² can also be a linker having 1-10 heteroatoms, preferably 1, 2, or 3 heteroatoms. Suitable heteroatom linkers include —O—, —S—, —NH—, —N═N—, and the like. For example, a suitable L¹ group is —SCH₂C(O)—.

In a further embodiment, R⁴ and L² together form a group selected from —N═N-aryl and —N═N-heteroaryl. Suitable examples of —N═N-aryl include, but are not limited to, —N═N-phenyl, in which the phenyl is optionally substituted, and -N=N-naphthyl, in which the naphthyl is optionally substituted.

In a further embodiment, R⁴ and L² together form a group selected from

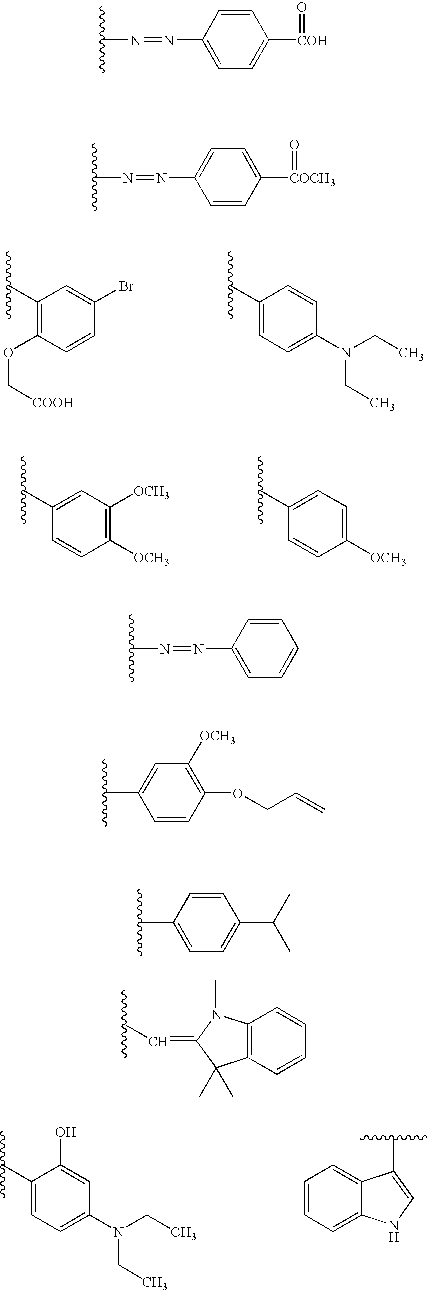

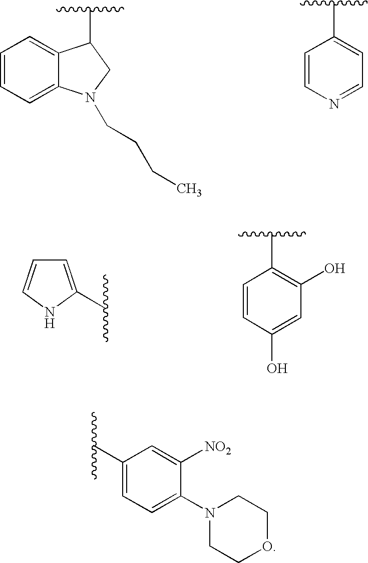

In a first subclass, the present invention is directed to a method of inhibiting a taste modulating protein, said method comprising contacting said protein with a compound of Formula I wherein

R¹ is optionally substituted C₆₋₁₀ to aryl;

R² is H or C₁₋₆ alkyl preferably C₁₋₄ alkyl

R³ is H or C₁₋₆ alkyl, preferably C₁₋₄ alkyl; and

R⁴ is optionally substituted C₆₋₁₀ aryl.

In one embodiment within this first subclass, R¹ is unsubstituted phenyl. In other instances, the C₆₋₁₀ aryl group, such as a phenyl group, is substituted with 1, 2, or 3 groups independently selected from the group consisting of amino, hydroxy, nitro, halogen, cyano, thiol, C₁₋₆ alkyl, C₂₋₆ alkenyl, C₂₋₆ alkynyl, C₁₋₆ haloalkyl, C₃₋₆ cycloalkyl, C₃₋₆ cycloalkenyl, C₃₋₆ cycloheteroalkyl, C₃₋₆ cycloheteroalkenyl, C₁₋₆ alkoxy, C₃₋₆ alkenyloxy, C₁₋₆ alkylthio, C₁₋₆ alkylenedioxy, C₁₋₆ alkoxy(C-₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, mono(C₁₋₄)alkylamino(C₂₋₆)alkoxy, di(Ci ₁₋₄)alkylamino(C₂₋₆)alkoxy, C₂₋₁₀ mono(carboxyalkyl)amino, bis(C₂₋₁₀ carboxyalkyl)amino, aminocarbonyl, C₂₋₆ alkynylcarbonyl, C₁₋₆ alkylsulfonyl, C₂₋₆ alkynylsulfonyl, C₁₋₆ alkylsulfinyl, C₁₋₆ alkylsulfonamido, C₆₋₁₀ arylsulfonamido, C₁₋₆ alkyliminoamino, formyliminoamino, C₂₋₆ carboxyalkoxy, C₂₋₆ carboxyalkyl, and carboxy(C₁₋₆)alkylamino.

In still further instances, the aryl group substituents are selected from the group consisting of amino, hydroxy, nitro, halogen, cyano, thiol, C₁₋₆ alkyl, C₂₋₆ alkenyl, C₁₋₆ haloalkyl, C₁₋₆ alkoxy, C₃₋₆ alkenyloxy, C₁₋₆ alkylenedioxy, C₁₋₆ alkoxy(C₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, C₂₋₆ carboxyalkoxy, and C₂₋₆ carboxyalkyl.

In another embodiment, the substituents on R¹ are independently selected from the group consisting of nitro, bromo, chloro, carboxy, methoxycarbonyl, methoxy, diethylamino, hydroxymethyl, methyl, allyloxy, trifluoromethylthio, hydroxy, trifluoromethyl, morpholinyl, and pyrrolidinyl.

In another embodiment within this first subclass, L¹ is a linker containing 1-6 carbon and/or heteroatoms and which is optionally substituted.

In another embodiment within this first subclass, L² is a linker containing 1-6 carbon and/or heteroatoms and which is optionally substituted.

In another embodiment within this first subclass, R⁴ is phenyl, optionally substituted with 1 to 3 substituents selected from the group consisting of nitro, bromo, chloro, carboxy, methoxycarbonyl, methoxy, diethylamino, hydroxymethyl, methyl, allyloxy, trifluoromethylthio, hydroxy, trifluoromethyl, morpholinyl, and pyrrolidinyl.

In a second subclass, the present invention is directed to a method of inhibiting a taste modulating protein, said method comprising contacting said protein with a compound of Formula I wherein

R¹ is optionally substituted 5-10 membered heteroaryl;

R² is H or C₁₋₆ alkyl;

R³ is H or C₁₋₆ alkyl; and

R⁴ is optionally substituted C₆₋₁₀ aryl.

In one embodiment within this second subclass, R¹ is an unsubstituted 5-10 membered heteroaryl, such as indolyl, pyridyl, benzothiazolyl, benzimidazolyl, or quinolinyl. Alternatively, R¹ is 5-10 membered heteroaryl subsituted with one or more substituents independently selected from the group consisting of amino, hydroxy, nitro, halogen, cyano, thiol, C₁₋₆ alkyl, C₂₋₆ alkenyl, C₂₋₆ alkynyl, C₁₋₆ haloalkyl, C₃₋₆ cycloalkyl, C₃₋₆ cycloalkenyl, C₃₋₆ cycloheteroalkyl, C₃₋₆ cycloheteroalkenyl, C₁₋₆ alkoxy, C₃₋₆ alkenyloxy, C₁₋₆ alkylthio, C₁₋₆ alkylenedioxy, C₁₋₆ alkoxy(C₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, mono(C₁₋₄)alkylamino(C₂₋₆)alkoxy, di(C₁₋₄)alkylamino(C₂₋₆)alkoxy, C₂₋₁₀ mono(carboxyalkyl)amino, bis(C₂₋₁₀ carboxyalkyl)amino, aminocarbonyl, C₂₋₆ alkynylcarbonyl, C₁₋₆ alkylsulfonyl, C₂₋₆ alkynylsulfonyl, C₁₋₆ alkylsulfinyl, C₁₋₆ alkylsulfonamido, C₆₋₁₀ arylsulfonamido, C₁₋₆ alkyliminoamino, formyliminoamino, C₂₋₆ carboxyalkoxy, C₂₋₆ carboxyalkyl, and carboxy(C₁₋₆)alkylamino.

In still further instances, the heteroaryl substituents are selected from the group consisting of amino, hydroxy, nitro, halogen, cyano, thiol, C₁₋₆ alkyl, C₂₋₆ alkenyl, C₁₋₆ haloalkyl, C₁₋₆ alkoxy, C₃₋₆ alkenyloxy, C₁₋₆ alkylenedioxy, C₁₋₆ alkoxy(C₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, C₂₋₆ carboxyalkoxy, and C₂₋₆ carboxyalkyl.

In another embodiment, the substituents on R¹ are independently selected from the group consisting of nitro, bromo, chloro, carboxy, methoxycarbonyl, methoxy, diethylamino, hydroxymethyl, methyl, allyloxy, trifluoromethylthio, hydroxy, trifluoromethyl, morpholinyl, and pyrrolidinyl.

In another embodiment within this first subclass, L¹ is a linker containing 1-10, preferably 1-4 carbon and/or heteroatoms and which is optionally substituted.

In another embodiment within this first subclass, L² is a linker containing 1-10, preferably 1-4 carbon and/or heteroatoms and which is optionally substituted.

In a third subclass, the present invention is directed to a method of inhibiting a taste modulating protein, said method comprising contacting said protein with a compound of Formula I wherein

R¹ is optionally substituted C₆₋₁₀ aryl;

R² is H or C₁₋₆ alkyl;

R³ is H or C₁₋₆ alkyl; and

R⁴ is optionally substituted 5-10 membered heteroaryl;

In one embodiment within this third subclass, R¹ is unsubstituted phenyl. In other instances, the C₆₋₁₀ aryl group, such as a phenyl group, is substituted with 1, 2, or 3 groups independently selected from the group consisting of amino, hydroxy, nitro, halogen, cyano, thiol, C₁₋₆ alkyl, C₂₋₆ alkenyl, C₂₋₆ alkynyl, C₁₋₆ haloalkyl, C₃₋₆ cycloalkyl, C₃₋₆ cycloalkenyl, C₃₋₆ cycloheteroalkyl, C₃₋₆ cycloheteroalkenyl, C₁₋₆ alkoxy, C₃₋₆ alkenyloxy, C₁₋₆ alkylthio, C₁₋₆ alkylenedioxy, C₁₋₆ alkoxy(C₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, mono(C₁₋₄)alkylamino(C₂₋₆)alkoxy, di(C₁₋₄)alkylamino(C₂₋₆)alkoxy, C₂₋₁₀ mono(carboxyalkyl)amino, bis(C₂₋₁₀ carboxyalkyl)amino, aminocarbonyl, C₂₋₆ alkynylcarbonyl, C₁₋₆ alkylsulfonyl, C₂₋₆ alkynylsulfonyl, C₁₋₆ alkylsulfinyl, C₁₋₆ alkylsulfonamido, C₆₋₁₀ arylsulfonamido, C₁₋₆ alkyliminoamino, formyliminoamino, C₂₋₆ carboxyalkoxy, C₂₋₆ carboxyalkyl, and carboxy(C₁₋₆)alkylamino.

In still further instances, the aryl group substituents are selected from a group consisting of amino, hydroxy, nitro, halogen, cyano, thiol, C₁₋₆ alkyl, C₂₋₆ alkenyl, C₁₋₆ haloalkyl, C₁₋₆ alkoxy, C₃₋₆ alkenyloxy, C₁₋₆ alkylenedioxy, C₁₋₆ alkoxy(C₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, C₂₋₆ carboxyalkoxy, and C₂₋₆ carboxyalkyl.

In another embodiment, the substituents on R¹ are independently selected from the group consisting of nitro, bromo, chloro, carboxy, methoxycarbonyl, methoxy, diethylamino, hydroxymethyl, methyl, allyloxy, trifluoromethylthio, hydroxy, trifluoromethyl, morpholinyl, and pyrrolidinyl.

In another embodiment within this first subclass, L¹ is a linker containing 1-10, preferably 1-4, carbon and/or heteroatoms and which is optionally substituted.

In another embodiment within this first subclass, L2 is a linker containing 1-10, preferably 1-4, carbon and/or heteroatoms and which is optionally substituted.

In one embodiment within this third subclass, R⁴ is an unsubstituted 5-10 membered heteroaryl, such as indolyl, pyridyl, benzothiazolyl, benzimidazolyl, or quinolinyl. Alternatively, R¹ is 5-10 membered heteroaryl subsituted with one or more substituents independently selected from the group consisting of nitro, bromo, chloro, carboxy, methoxycarbonyl, methoxy, diethylamino, hydroxymethyl, methyl, allyloxy, trifluoromethylthio, hydroxy, trifluoromethyl, morpholinyl, and pyrrolidinyl.

In a fourth subclass, the present invention is directed to a method of inhibiting a taste modulating protein, said method comprising contacting said protein with a compound of Formula I wherein

R¹ is optionally substituted 5-10 membered heteroaryl;

R² is H or C₁₋₆ alkyl;

R³ is H or C₁₋₆ alkyl; and

R⁴ is optionally substituted 5-10 membered heteroaryl.

In one embodiment within this fourth subclass, R¹ is an unsubstituted 5-10 membered heteroaryl, such as indolyl, pyridyl, or quinolinyl. Alternatively, R¹ is 5-10 membered heteroaryl subsituted with one or more substituents independently selected from the group consisting of amino, hydroxy, nitro, halogen, cyano, thiol, C₁₋₆ alkyl, C₂₋₆ alkenyl, C₂₋₆ alkynyl, C₁₋₆ haloalkyl, C₃₋₆ cycloalkyl, C₃₋₆ cycloalkenyl, C₃₋₆ cycloheteroalkyl, C₃₋₆ cycloheteroalkenyl, C₁₋₆ alkoxy, C₃₋₆ alkenyloxy, C₁₋₆ alkylthio, C₁₋₆ alkylenedioxy, C₁₋₆ alkoxy(C₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, mono(C₁₋₄)alkylamino(C₂₋₆)alkoxy, di(C₁₋₄)alkylamino(C₂₋₆)alkoxy, C₂₋₁₀ mono(carboxyalkyl)amino, bis(C₂₋₁₀ carboxyalkyl)amino, aminocarbonyl, C₂₋₆ alkynylcarbonyl, C₁₋₆ alkylsulfonyl, C₂₋₆ alkynylsulfonyl, C₁₋₆ alkylsulfinyl, C₁₋₆ alkylsulfonamido, C₆₋₁₀ arylsulfonamido, C₁₋₆ alkyliminoamino, formyliminoamino, C₂₋₆ carboxyalkoxy, C₂₋₆ carboxyalkyl, and carboxy(C₁₋₆)alkylamino.

In still further instances, the heteroaryl substituents are selected from a group consisting of amino, hydroxy, nitro, halogen, cyano, thiol, C₁₋₆ alkyl, C₂₋₆ alkenyl, C₁₋₆ haloalkyl, C₁₋₆ alkoxy, C₃₋₆ alkenyloxy, C₁₋₆ alkylenedioxy, C₁₋₆ alkoxy(C₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄)alkylamino, di(Ci ₁₋₄)alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, C₂₋₆ carboxyalkoxy, and C₂₋₆ carboxyalkyl.

In another embodiment, the substituents on R¹ are independently selected from the group consisting of nitro, bromo, chloro, carboxy, methoxycarbonyl, methoxy, diethylamino, hydroxymethyl, methyl, allyloxy, trifluoromethylthio, hydroxy, trifluoromethyl, morpholinyl, and pyrrolidinyl.

In one embodiment within this fourth subclass, R⁴ is an unsubstituted 5-10 membered heteroaryl, such as indolyl, pyridyl, benzothiazolyl, benzimidazolyl or quinolinyl. Alternatively, R¹ is a 5-10 membered heteroaryl subsituted with one or more substituents independently selected from the group consisting of amino, hydroxy, nitro, halogen, cyano, thiol, C₁₋₆ alkyl, C₂₋₆ alkenyl, C₂₋₆ alkynyl, C₁₋₆ haloalkyl, C₃₋₆ cycloalkyl, C₃₋₆ cycloalkenyl, C₃₋₆ cycloheteroalkyl, C₃₋₆ cycloheteroalkenyl, C₁₋₆ alkoxy, C₃₋₆ alkenyloxy, C₁₋₆ alkylthio, C₁₋₆ alkylenedioxy, C₁₋₆ alkoxy(C₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, mono(C₁₋₄)alkylamino(C₂₋₆)alkoxy, di(C₁₋₄)alkylamino(C₂₋₆)alkoxy, C₂₋₁₀ mono(carboxyalkyl)amino, bis(C₂₋₁₀ carboxyalkyl)amino, aminocarbonyl, C₂₋₆ alkynylcarbonyl, C₁₋₆ alkylsulfonyl, C₂₋₆ alkynylsulfonyl, C₁₋₆ alkylsulfinyl, C₁₋₆ alkylsulfonamido, C₆₋₁₀ arylsulfonamido, C₁₋₆ alkyliminoamino, formyliminoamino, C₂₋₆ carboxyalkoxy, C₂₋₆ carboxyalkyl, and carboxy(C₁₋₆)alkylamino.

In still further instances, the heteroaryl substituents are selected from a group consisting of amino, hydroxy, nitro, halogen, cyano, thiol, C₁₋₆ alkyl, C₂₋₆ alkenyl, C₁₋₆ haloalkyl, C₁₋₆ alkoxy, C₃₋₆ alkenyloxy, C₁₋₆ alkylenedioxy, C₁₋₆ alkoxy(C₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, C₂₋₆ carboxyalkoxy, and C₂₋₆ carboxyalkyl.

In another embodiment, the substituents on R⁴ are independently selected from the group consisting of nitro, bromo, chloro, carboxy, methoxycarbonyl, methoxy, diethylamino, hydroxymethyl, methyl, allyloxy, trifluoromethylthio, hydroxy, trifluoromethyl, morpholinyl, and pyrrolidinyl.

In a fifth subclass, the present invention is directed to a method of inhibiting a taste modulating protein, said method comprising contacting said protein with a compound of Formula I wherein

R¹ is optionally substituted C₆₋₁₀ aryl;

R² is H or C₁₋₆ alkyl;

R³ is H or C₁₋₆ alkyl; and

R⁴ is optionally substituted C₃₋₁₀ cycloalkyl.

In one embodiment within this fifth subclass, R¹ is unsubstituted phenyl. In other instances, the C₆₋₁₀ aryl group, such as a phenyl group, is substituted with 1, 2, or 3 groups independently selected from the group consisting of amino, hydroxy, nitro, halogen, cyano, thiol, C₁₋₆ alkyl, C₂₋₆ alkenyl, C₂₋₆ alkynyl, C₁₋₆ haloalkyl, C₃₋₆ cycloalkyl, C₃₋₆ cycloalkenyl, C₃₋₆ cycloheteroalkyl, C₃₋₆ cycloheteroalkenyl, C₁₋₆ alkoxy, C₃₋₆ alkenyloxy, C₁₋₆ alkylthio, C₁₋₆ alkylenedioxy, C₁₋₆ alkoxy(C₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, carboxy, (C₁₋₆) alkoxy(C₂₋₆)alkoxy, mono(C₁₋₄)alkylamino(C₂₋₆)alkoxy, di(C₁₋₄)alkylamino(C₂₋₆)alkoxy, C₂₋₁₀ mono(carboxyalkyl)amino, bis(C₂₋₁₀ carboxyalkyl)amino, aminocarbonyl, C₂₋₆ alkynylcarbonyl, C₁₋₆ alkylsulfonyl, C₂₋₆ alkynylsulfonyl, C₁₋₆ alkylsulfinyl, C₁₋₆ alkylsulfonamido, C₆₋₁₀ arylsulfonamido, C₁₋₆ alkyliminoamino, formyliminoamino, C₂₋₆ carboxyalkoxy, C₂₋₆ carboxyalkyl, and carboxy(C₁₋₆)alkylamino.

In still further instances, the aryl substituents are selected from a group consisting of amino, hydroxy, nitro, halogen, cyano, thiol, C₁₋₆ alkyl, C₂₋₆ alkenyl, C₁₋₆ haloalkyl, C₁₋₆ alkoxy, C₃₋₆ alkenyloxy, C₁₋₆alkylenedioxy, C₁₋₆ alkoxy(C₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, C₂₋₆ carboxyalkoxy, and C₂₋₆ carboxyalkyl.

In another embodiment, the substituents on R¹ are independently selected from the group consisting of nitro, bromo, chloro, carboxy, methoxycarbonyl, methoxy, diethylamino, hydroxymethyl, methyl, allyloxy, trifluoromethylthio, hydroxy, trifluoromethyl, morpholinyl, and pyrrolidinyl.

In a sixth subclass, the present invention is directed to a method of inhibiting a taste modulating protein, said method comprising contacting said protein with a compound of Formula I wherein

R¹ is optionally substituted 5-10 membered heteroaryl;

R² is H or C₁₋₆ alkyl;

R³ is H or C₁₋₆ alkyl; and

R⁴ and L² together form —N═N-aryl.

In one embodiment within this sixth subclass, R¹ is an unsubstituted 5-10 membered heteroaryl, such as indolyl, pyridyl, or quinolinyl. Alternatively, R¹ is a 5-10 membered heteroaryl subsituted with one or more substituents independently selected from the group consisting of amino, hydroxy, nitro, halogen, cyano, thiol, C₁₋₆ alkyl, C₂₋₆ alkenyl, C₂₋₆ alkynyl, C₁₋₆ haloalkyl, C₃₋₆ cycloalkyl, C₃₋₆ cycloalkenyl, C3-₆ cycloheteroalkyl, C₃₋₆ cycloheteroalkenyl, C₁₋₆ alkoxy, C₃₋₆ alkenyloxy, C₁₋₆ alkylthio, C₁₋₆ alkylenedioxy, C₁₋₆ alkoxy(C₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄) alkylamino, di(C₁₋₄) alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, mono(C₁₋₄)alkylamino(C₂₋₆)alkoxy, di(C₁₋₄)alkylamino(C₂₋₆)alkoxy, C₂₋₁₀ mono(carboxyalkyl)amino, bis(C₂₋₁₀ carboxyalkyl)amino, aminocarbonyl, C₂₋₆ alkynylcarbonyl, C₁₋₆ alkylsulfonyl, C₂₋₆ alkynylsulfonyl, C₁₋₆ alkylsulfinyl, C₁₋₆ alkylsulfonamido, C₆₋₁₀ arylsulfonamido, C₁₋₆ alkyliminoamino, formyliminoamino, C₂₋₆ carboxyalkoxy, C₂₋₆ carboxyalkyl, and carboxy(C₁₋₆)alkylamino. In another embodiment, the substituents on R¹ are independently selected from the group consisting of nitro, bromo, chloro, carboxy, methoxycarbonyl, methoxy, diethylamino, hydroxymethyl, methyl, allyloxy, trifluoromethylthio, hydroxy, trifluoromethyl, morpholinyl, and pyrrolidinyl.

In this sixth subclass, R⁴ and L² together form —N═N-aryl, wherein aryl is a C₆₋₁₀ optionally substituted aryl group, such as phenyl or naphthyl. Suitable substituents on the aryl group include, but are not limited to, nitro, bromo, chloro, carboxy, methoxycarbonyl, methoxy, diethylamino, hydroxymethyl, methyl, allyloxy, trifluoromethylthio, hydroxy, trifluoromethyl, morpholinyl, and pyrrolidinyl.

In a seventh subclass, the present invention is directed to a method of inhibiting a taste modulating protein, said method comprising contacting said protein with a compound of Formula I wherein

R¹ is optionally substituted 5-10 membered heteroaryl, such as pyridyl, quinolinyl, benzothiazolyl, benzimidazolyl and indolyl;

R⁴ is optionally substituted C₆₋₁₀ aryl, such as phenyl and naphthyl; and

L¹ and L² are absent.

In an eighth subclass, the present invention is directed to a method of inhibiting a taste modulating protein, said method comprising contacting said protein with a compound of Formula I wherein

R¹ is C₆₋₁₀ aryl, 5-10 membered heteroaryl, C₃₋₁₀ cycloalkyl, C₃₋₁₀ cycloalkenyl, 3-10 membered cycloheteroalkyl, 3-10 membered cycloheteroalkenyl, and C₁₋₆ alkyl, each of which is optionally substituted;

R² is H, C₁₋₆ alkyl, or C₆₋₁₀ aryl(C₁₋₆)alkyl;

L¹ is absent, or is a linker containing 1-10, preferably 1-6, carbon and/or heteroatoms and which is optionally substituted;

R³, R⁴, and L² together with the carbon atom form a group selected from C₆₋₁₀ aryl, 5-10 membered heteroaryl, C₃₋₁₀ cycloalkyl, C₃₋₁₀ cycloalkenyl, 3-10 membered cycloheteroalkyl, 3-10 membered cycloheteroalkenyl, each of which is optionally substituted.

In an eighth subclass, the present invention is directed to a method of inhibiting a taste modulating protein, said method comprising contacting said protein with a compound of Formula I wherein

R¹ is optionally substituted indolinyl;

R² is H, C₁₋₆ alkyl, or C₆₋₁₀ aryl(C₁₋₆)alkyl;

L¹ is absent, or is a linker containing 1-10, preferably 1-6, carbon and/or heteroatoms and which is optionally substituted;

R³, R⁴, and L² together with the carbon atom form a group selected from C₆₋₁₀ aryl, 5-10 membered heteroaryl, C₃₋₁₀ cycloalkyl, C₃₋₁₀ cycloalkenyl, 3-10 membered cycloheteroalkyl, 3-10 membered cycloheteroalkenyl, each of which is optionally substituted.

In a further subclass, the invention is directed to a method of inhibiting a taste modulating protein, said method comprising contacting said protein with a compound of Formula I wherein R¹ is heteroaryl; R² is H; R⁴ is heteroaryl; L¹ is absent; and L² is N═N.

In a further subclass, the invention is directed to a method of inhibiting a taste modulating protein, said method comprising contacting said protein with a compound of Formula I wherein R¹ is a bicycloalkyl; R² is H; R³ is H;

R⁴ is aryl or heteroaryl; L¹ is absent; and L² is absent.

In a further subclass, the invention is directed to a method of inhibiting a taste modulating protein, said method comprising contacting said protein with a compound of Formula I wherein R¹ is aryl; R² is H; R³ is H; R⁴ is aryl or heteroaryl; L¹ is an optionally substituted a linker containing 2-4 carbon or hetero atoms; and L² is absent.

In a further subclass, the invention is directed to a method of inhibiting a taste modulating protein, said method comprising contacting said protein with a compound of Formula I wherein R¹ is cycloalkenyl; R² is H; R³ is H;

R⁴ is aryl or heteroaryl; L¹ is an optionally substituted a linker containing 2-4 carbon or hetero atoms; and L² is absent.

In a further subclass, the invention is directed to a method of inhibiting a taste modulating protein, said method comprising contacting said protein with a compound of Formula I wherein R¹ is optionally substituted aryl; R² is H; R³ is H; R⁴ is optionally substituted aryl or optionally substituted heteroaryl; L¹ is —(CH₂)₁₋₆ —C(O)—; and L² is absent.

In a further subclass, the invention is directed to a method of inhibiting a taste modulating protein, said method comprising contacting said protein with a compound of Formula I wherein R¹ is optionally substituted naphthyl;

R² is H; R³ is H; R⁴ is optionally substituted aryl; L¹ is —(CH₂)—C(O)—; and L² is absent.

Other suitable compounds for use in the methods of the invention include a compound according to Formula I wherein R¹ is phenyl substituted with amino, alkylamino, or dialkylamino, and R² is an optionally substituted benzo[d][1,3]dioxol-5-yl group; wherein R¹ is a C3-6 cycloalky optionally substituted with hydroxy, and R² is phenyl optionally substituted with one or more hyrdroxy and/or C₁₋₄ alkoxy; wherein R¹ is phenyl and R⁴ is phenyl optionally substituted with one or more groups selected from hydroxy, amino, alkylamino, and dialkylamino; or wherein R¹ is 3-indolyl and R⁴ is phenyl optionally substituted with 1-4 C₁₋₄ alkoxy groups.

In a further subclass, the invention is directed to the use of a compound according to Formula I wherein R¹ is optionally substituted phenyl; R² is optionally substituted phenyl; L¹ is a C₃₋₅ linker, such as one containing a cyclopropyl group; and L² is absent. A subgroup of compounds within this subclass are compounds according to the following Formula II

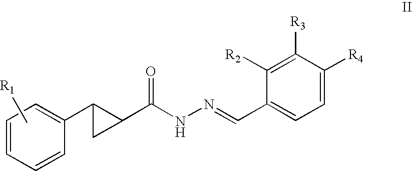

wherein R₁ is hydrogen or halogen; R₂ is hydrogen or C₁₋₄ haloalkyl;

R₃ is hydrogen, C₁₋₄ haloalkyl, C₁₋₄ alkoxy, or C₁₋₄ alkylthio; and R₄ is hydrogen, C₁₋₄ haloalkyl, C₁₋₄ alkoxy, or C₁₋₄ alkylthio. In another embodiment, R₁ is hydrogen or halogen; R₂ is CF₃; R₃ is hydrogen, C₁₋₄ haloalkyl, C₁₋₄ alkoxy, or C₁₋₄ alkylthio; and R₄ is hydrogen, C₁₋₄ haloalkyl, C₁₋₄ alkoxy, or C₁₋₄ alkylthio. Suitable alkoxy groups include methoxy. Suitable haloalkyl groups include trifluoromethoxy. Suitable alkylthio groups include —SCH₃. Preferably, the compounds are trans-cyclopropyl compounds. Examples of compounds of the present invention are described herein, for example in the Examples.

Examples of suitable compounds for use in the method of the present invention include:

methyl 4-((E)-((Z)-1-(2-(benzo[d]thiazol-2-yl)hydrazono)-2-methyl-propyl)diazenyl)benzoate;

(E)-2-(4-bromo-2-((2-(quinolin-8-yl)hydrazono)methyl)phenoxy)-acetic acid;

(E)-N′-(3,4-dimethoxybenzylidene)-2-(naphthalene-1 -yl)-acetohydrazide;

(E)-N′-(3,4-dimethoxybenzylidene)-2-phenylcyclopropane-carbohydrazide;

(E)-3 -cyclohexenyl-4-hydroxy-N′-(4-methoxybenzylidene)-butanehydrazide;

(E)-N′-(3,4-dimethoxybenzylidene)-4-hydroxyhexanehydrazide;

2-((Z)-2-(phenyl((E)-phenyldiazenyl)methylene)hydrazinyl)benzoic acid;

(E)-N′-(3,4-dimethoxybenzylidene)-2-(m-tolyloxy)acetohydrazide;

(E)-N′-(4-(allyloxy)-3 -methoxybenzylidene)-2-(3 -bromobenzylthio)-acetohydrazide;

(E)-N′-(4-isopropylbenzylidene)bicyclo[4.1.0]heptane-7-carbo-hydrazide;

(Z)-1,3,3-trimethyl-2-((E)-2-(2-(4-nitrophenyl)hydrazono)-ethylidene)indoline;

(E)-N′-(4-(diethylamino)-2-hydroxybenzylidene)-2-phenylcyclo-propanecarbohydrazide;

(4-(trifluoromethylthio)phenyl)carbonohydrazonoyldicyanide;

N-((E)-3-((Z)-2-(1,5-dimethyl-2-oxoindolin-3-ylidene)hydrazinyl)-3-oxo-1-phenylprop-1-en-2-yl)benzamide;

(Z)-2-(2-((1 -butyl-1H-indol-3-yl)methylene)hydrazinyl)benzoic acid;

(E)-4-((2-benzyl-2-phenylhydrazono)methyl)pyridine;

(Z)-N′-((1H-pyrrol-2-yl)methylene)tricyclo[3.3.1.1^{3.7}]decane-3-carbohydrazide;

(Z)-1-(2-(4-(ethyl(2-hydroxyethyl)amino)phenyl)hydrazono)-naphthalen-2-(1 H)-one;

(E)-4-((2-(5-chloro-3-(trifluoromethyl)pyridini-2-yl)-2-2-methyl -hydrazono)methyl)benzene-1,3-diol;

(E)-2-(3,4-dimethylphenylamino)-N′(4-morpholino-3-nitrobenzylidene)acetohydrazide;

(Z)-3-(2-nitro-5-(pyrrolidin-1 -yl)phenyl)hydrazono)quinuclidine;

(E)-2-((2-(1H-benzo[d]imidazol-2-yl)hydrazono)methyl)-5-(diethylamino)phenol;

and physiologically acceptable salts thereof.

Examples of suitable compounds for use in the method of the present invention include:

N-(3-(2-((6-Bromobenzo[d][1,3]dioxol-5-yl)methylene)hydrazinyl)-1-(dimethylamino)phenyl)-3-oxoprop-1-en-2-yl)benzamide;

N-(1-(4-(Diethylamino)phenyl)-3-(2-(4-hydroxy-3-iodo-5-methoxybenzylidene)hydrazinyl)-3-oxoprop-1-en-2-yl)benzamide;

N′-(4-Hydroxy-3-methoxybenzylidene)-3-(1-hydroxycyclopentyl)-propanehydrazide;

4-Nitro-N′-(3,4,5-trimethoxybenzylidene)benzohydrazide;

N′-(4-(diethylamino)-2-hydroxybenylidine)phenylcyclopropane-carboxhydrazide;

N′-(5-Bromo-2-oxoindolin-3-ylidene)-2-(2-bromo-4-methoxyphenoxy)acetohydrazide;

3-(1 H-indol-3-yl)-N′-(3,4,5-trimethoxybenzylidene)propanehydrazide;

N′-(2-oxoindolin-3-ylidene)-2-(2-methyl-4-(1,1-dimethylethyl)-phenoxy)acetohydrazide;

2-(4-chlorophenyl)-N′-(3,4-dimethoxybenylidine)cyclopropane-carboxhydrazide;

2-(2-chlorophenyl)-N′-(3,4-dimethoxybenylidine)cyclopropane-carboxhydrazide;

2-(3-chlorophenyl)-N′-(3,4-dimethoxybenylidine)cyclopropane-carboxhydrazide;

2-(2-fluorophenyl)-N′-(3,4-dimethoxybenylidine)cyclopropane-carboxhydrazide;

2-(3-fluorophenyl)-N′-(3,4-dimethoxybenylidine)cyclopropane-carboxhydrazide;

2-(4-fluorophenyl)-N′-(3,4-dimethoxybenylidine)cyclopropane-carboxhydrazide;

2-(2-chlorophenyl)-N′-(3-trifluoromethylbenylidine)cyclopropane-carboxhydrazide;

2-(3-chlorophenyl)-N′-(3-trifluoromethylbenylidine)cyclopropane-carboxhydrazide;

2-(4-chlorophenyl)-N′-(3-trifluoromethylbenylidine)cyclopropane-carboxhydrazide;

2-(2-fluorophenyl)-N′-(3-trifluoromethylbenylidine)cyclopropane-carboxhydrazide;

2-(3-fluorophenyl)-N′-(3-trifluoromethylbenylidine)cyclopropane-carboxhydrazide;

2-(4-fluorophenyl)-N′-(3-trifluoromethylbenylidine)cyclopropane-carboxhydrazide;

2-(2-chlorophenyl)-N′-(3-methoxybenylidine)cyclopropane-carboxhydrazide;

2-(3-chlorophenyl)-N′-(3-methoxybenylidine)cyclopropane-carboxhydrazide;

2-(4-chlorophenyl)-N′-(3-methoxybenylidine)cyclopropane-carboxhydrazide;

2-(2-fluorophenyl)-N′-(3-methoxybenylidine)cyclopropane-carboxhydrazide;

2-(3-fluorophenyl)-N′-(3-methoxybenylidine)cyclopropane-carboxhydrazide;

2-(4-fluorophenyl)-N′-(3-methoxybenylidine)cyclopropane-carboxhydrazide;

2-(2-chlorophenyl)-N′-(3-methylthiobenylidine)cyclopropane-carboxhydrazide;

2-(3-chlorophenyl)-N′-(3-methylthiobenylidine)cyclopropane-carboxhydrazide;

2-(4-chlorophenyl)-N′-(3-methylthiobenylidine)cyclopropane-carboxhydrazide;

2-(2-fluorophenyl)-N′-(3-methylthiobenylidine)cyclopropane-carboxhydrazide;

2-(3-fluorophenyl)-N′-(3-methylthiobenylidine)cyclopropane-carboxhydrazide;

2-(4-fluorophenyl)-N′-(3-methylthiobenylidine)cyclopropane-carboxhydrazide;

2-(2-chlorophenyl)-N′-(2-trifluoromethylbenylidine)cyclopropane-carboxhydrazide;

2-(3-chlorophenyl)-N′-(2-trifluoromethylbenylidine)cyclopropane-carboxhydrazide;

2-(4-chlorophenyl)-N′-(2-trifluoromethylbenylidine)cyclopropane-carboxhydrazide;

2-(2-fluorophenyl)-N′-(2-trifluoromethylbenylidine)cyclopropane-carboxhydrazide;

2-(3-fluorophenyl)-N′-(2-trifluoromethylbenylidine)cyclopropane-carboxhydrazide;

2-(4-fluorophenyl)-N′-(2-trifluoromethylbenylidine)cyclopropane-carboxhydrazide;

2-(2-chlorophenyl)-N′-(4-trifluoromethylbenylidine)cyclopropane-carboxhydrazide;

2-(3-chlorophenyl)-N′-(4-trifluoromethylbenylidine)cyclopropane-carboxhydrazide;

2-(4-chlorophenyl)-N′-(4-trifluoromethylbenylidine)cyclopropane-carboxhydrazide;

2-(2-fluorophenyl)-N′-(4-trifluoromethylbenylidine)cyclopropane-carboxhydrazide;

2-(3-fluorophenyl)-N′-(4-trifluoromethylbenylidine)cyclopropane-carboxhydrazide;

2-(4-fluorophenyl)-N′-(4-trifluoromethylbenylidine)cyclopropane-carboxhydrazide;

N′-(3,4-dimethoxybenzylidene)-2-(4,8-dimethylquinolin-2-ylthio)-acetohydrazide;

3-(9H-carbazol-9-yl)-N′-(3,4-dimethoxybenzylidene)propane-hydrazide;

and physiologically acceptable salts thereof

The methods of the present invention also include the use of a physiologically acceptable salt of a compound according to Formula I. The term physiologically acceptable salt refers to an acid- and/or base-addition salt of a compound according to Formula I. Acid-addition salts can be formed by adding an appropriate acid to the compound according to Formula I. Base-addition salts can be formed by adding an appropriate base to the compound according to Formula I. Said acid or base does not substantially degrade, decompose, or destroy said compound according to Formula I. Examples of suitable physiologically acceptable salts include hydrochloride, hydrobromide, acetate, furmate, maleate, oxalate, and succinate salts. Other suitable salts include sodium, potassium, carbonate, and tromethamine salts.

It is also to be understood that the present invention is considered to encompass the use of stereoisomers as well as optical isomers, e.g., mixtures of enantiomers as well as individual enantiomers and diastereomers, which arise as a consequence of structural asymmetry in selected compounds of the present series. It is further understood that the present invention encompasses the use of tautomers of a compound of Formula I. Tautomers are well-known in the art and include keto-enol tautomers.

It is also understood that the compounds of Formula I include both the E and Z isomers, in varying ratios, of the hydrazone. As is known in the art, the hydrazone moiety can isomerize between the E and Z isomers, as shown in the following schematic:

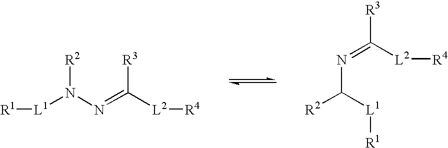

While the specific compounds listed above may indicate a particular stereochemistry of the hydrazone moiety, i.e., E or Z, the present invention explicitly includes both isomers.

The compounds of Formula I may also be solvated, including hydrated. Hydration may occur during manufacturing of the compounds or compositions comprising the compounds, or the hydration may occur over time due to the hygroscopic nature of the compounds.

Certain compounds within the scope of Formula I may be derivatives referred to as “prodrugs.” The expression “prodrug” denotes a derivative of a known direct acting agent, wherein the derivative has therapeutic value that may be similar to, greater than, or less than that of the agent. Generally, the prodrug is transformed into the active agent by an enzymatic or chemical process when delivered to the subject, cell, or test media. In certain instances, prodrugs are derivatives of the compounds of the invention which have metabolically cleavable groups and become by solvolysis or under physiological conditions the compounds of the invention which are pharmaceutically active in vivo. For example, ester derivatives of compounds of this invention are often active in vivo, but not in vitro. Other derivatives of the compounds of this invention have activity in both their acid and acid derivative forms, but the acid derivative form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with an amine. Simple aliphatic or aromatic esters derived from acidic groups pendent on the compounds of this invention are preferred prodrugs. In some cases, it is desirable to prepare double ester type prodrugs such as (acyloxy) alkyl esters or ((alkoxycarbonyl)oxy)alkyl esters.

When any variable occurs more than one time in any constituent or in Formula I, its definition on each occurrence is independent of its definition at every other occurrence, unless otherwise indicated- Also, combinations of substituents and/or variables are permissible only if such combinations result in stable compounds.

The term “alkyl,” as used herein by itself or as part of another group, refers to both straight and branched chain radicals of up to 10 carbons, unless the chain length is limited thereto, such as methyl, ethyl, propyl, isopropyl, butyl, 1-methylpropyl, 2-methylpropyl, pentyl, 1-methylbutyl, isobutyl, pentyl, t-amyl (CH₃CH₂(CH₃)₂C—), hexyl, isohexyl, heptyl, octyl, or decyl.

The term “alkenyl,” as used herein by itself or as part of another group, refers to a straight or branched chain radical of 2-10 carbon atoms, unless the chain length is limited thereto, including, but not limited to, ethenyl, 1-propenyl, 2-propenyl, 2-methyl-1-propenyl, 1-butenyl, 2-butenyl, 3-butenyl, pentenyl, 1-hexenyl, and 2-hexenyl.

The term “alkynyl,” as used herein by itself or as part of another group, refers to a straight or branched chain radical of 2-10 carbon atoms, unless the chain length is limited thereto, wherein there is at least one triple bond between two of the carbon atoms in the chain, including, but not limited to, ethynyl, 1-propynyl, 2-propynyl, 1-butynyl, 2-butynyl, 1-methyl-2-butynyl, 1-methyl-3-butynyl, 2-methyl-3-pentynyl, hexynyl, and heptynyl.

In instances herein where there is an alkenyl or alkynyl moiety as a substituent group, the unsaturated linkage is preferably not directly attached to a nitrogen, oxygen or sulfur moiety.

The term “cycloalkyl,” as used herein by itself or as part of another group, refers to cycloalkyl groups containing 3 to 14, preferably 3 to 10, carbon atoms. Typical examples are cyclopropyl, cyclobutyl, cyclopentyl, and cyclohexyl. Cycloalkyl also includes bicycloalkyl, polycycloalkyl, and other bridged cycloalkyl groups.

The term “cycloalkenyl,” as used herein by itself or as part of another group, refers to cycloalkenyl groups containing 3 to 10, carbon atoms and 1 to 3 carbon-carbon double bonds. Typical examples include cyclopropenyl, cyclobutenyl, cyclopentenyl, cyclohexenyl, cycloheptenyl, and cyclohexadienyl. Cycloalkenyl also includes bicycloalkenyl, polycycloalkenyl, and other bridged cycloalkenyl groups.

The term “cycloheteroalkyl,” as employed herein by itself or as part of another group, refers to a group having 3 to 14 ring atoms containing carbon atoms and 1, 2, 3, or 4 oxygen, nitrogen, or sulfur heteroatoms. Typical examples include, but are not limited to, 2-tetrahydrofuranyl, 2-tetrahydrothienyl, 2-pyrrolidinyl, 3-isoxazolidinyl, 3-isothiazolidinyl, 1,3,4-oxazolidin-2-yl, 2,3-dihydrothien-2-yl, 4,5-isoxazolin-3-yl, 3-piperidinyl, 1,3-dioxan-5-yl, 4-piperidinyl, 2-tetrahydropyranyl, 4-tetrahydropyranyl, pyrrolidinyl, imidazolidinyl, pirazolidinyl, tetrahydrofuranyl, tetrahydropyranyl, piperidyl, piperazinyl, quinuclidinyl, and morpholinyl.

The term “cycloheteroalkenyl,” as used by itself or as part of another group, refers to a group containing 3 to 14 ring atoms containing carbon atoms and 1, 2, 3, or 4 oxygen, nitrogen, or sulfur atoms and 1, 2, or 3 double bonds.

Typical examples include preferably the cycloheteroalkyl groups recited above, specifically pyrrolidinyl, imidazolidinyl, pirazolidinyl, tetrahydrofuranyl, tetrahydropyranyl, piperidyl, piperazinyl, quinuclidinyl, and morpholinyl, and modified so as to contain 1 or 2 double bonds.

The term “alkylene,” as used herein by itself or as a part of another group, refers to a diradical of an unbranched saturated hydrocarbon chain, having, unless otherwise indicated, from 1 to 15 carbon atoms, preferably 1 to 10 carbon atoms and more preferably 1 to 6 carbon atoms. This term is exemplified by groups such as methylene (—CH═CH₂—), ethylene (—CH₂CH₂—), propylene (—CH₂CH₂CH₂—), butylene, and the like.

The term “alkenylene,” as used herein by itself or part of another group, refers to a diradical of an unbranched, unsaturated hydrocarbon chain, having, unless otherwise indicated, from 2 to 15 carbon atoms, preferably 1 to 10 carbon atoms, more preferably 1 to 6 carbon atoms, and having at least 1 and preferably from 1 to 6 sites of vinyl unsaturation. This term is exemplified by groups such as ethenylene (—CH═CH—), propenylene (—CH₂CH═CH—, —CH═CHCH₂—), and the like.

The term “alkynylene,” as used herein by itself or part of another group, refers to a diradical of an unbranched, unsaturated hydrocarbon having, unless otherwise indicated, from 2 to 15 carbon atoms preferably 1 to 10 carbon atoms, more preferably 1 to 6 carbon atoms, and having at least 1 and preferably from 1 to 6 sites of acetylene (triple bond) unsaturation. Examples include alkynylene groups such as ethynylene (—C≡C—), propargylene (—CH₂—C≡—C—), and the like.

The term “heteroalkylene,” as used herein by itself or party of another group means alkylene, as defined above, wherein 1 to 5 of the carbon atoms indicated is replaced by a heteroatom chosen from N, O, or S (e.g., amino, oxy, thio, aminomethylene (—NHCH₂—), oxymethylene (—OCH₂—), etc.). Examples include alkyleneoxy, alkyleneamino, and alkylenethio. Preferably, the oxygen, nitrogen, and sulfur atoms contained therein do not form bonds with other heteroatoms. Suitable groups include ethyleneoxy, propyleneoxy, butyleneoxy, pentyleneoxy, heptyleneoxy, ethyleneamino, propyleneamino, butyleneamino, pentyleneamino, hexyleneamino, heptyleneamino, and octyleneamino. Further examples include —CH₂CH₂—S—CH₂CH₂—and —CH₂—S—CH₂CH₂—NH-CH₂—. In one embodiment of heteroalkylene groups, heteroatoms can also occupy either but not both of the chain termini.

The term “heteroalkenylene,” as used herein by itself or part of another group, means alkenylene, as defined above, wherein 1 to 5 of the carbon atoms indicated is replaced by a heteroatom chosen from N, O, or S. Examples include alkenyleneoxy, alkenyleneamino, and alkenylenethio. Preferably, the oxygen, nitrogen, and sulfur atoms contained therein do not form bonds with other heteroatoms. Suitable groups include ethenyleneoxy, propenyleneoxy, butyenleneoxy, pentenyleneoxy, hexenyleneoxy, ethenyleneamino, propenyleneamino, butyenleneamino, pentenyleneamino, and hexenyleneamino. In one embodiment of heteroalkenylene groups, heteroatoms can also occupy either, but not both, of the chain termini.

Additionally, in another embodiment, the heteroatom does not form part of the vinyl bond.

The term “heteroalkynylene,” as used herein by itself or as part of another group, means alkynylene, as defined above, wherein 1 to 5 of the carbon atoms indicated is replaced by a heteroatom chosen from N, O, or S. Examples include alkynyleneoxy, alkynyleneamino, and alkynylenethio. Preferably, the oxygen, nitrogen, and sulfur atoms contained therein do not form bonds with other heteroatoms. In one embodiment of heteroalkynylene groups, heteroatoms can occupy either, but not both, of the chain termini. Additionally, the heteroatom does not form part of the vinyl bond.

The term “cycloalkylene,” as used herein by itself or as part of another group, refers to a non-aromatic alicyclic divalent hydrocarbon radical having from 3 to 15 carbon atoms, preferably 3 to 10 carbon atoms. Examples of “cycloalkylene” as used herein include, but are not limited to, cyclopropyl-1,1-diyl, cyclopropyl-1,2-diyl, cyclobutyl-1,2-diyl, cyclopentyl-1,3-diyl, cyclohexyl-1,4-diyl, and the like. Further examples include divalent groups which also contain an alkylene group such as methylenecyclopropylene (i.e., —CH₂-cyclopropylene-), ethylenecyclopropylene (i.e., —CH₂CH₂-cyclopropylene-), and methylenecyclohexylene (i.e., —CH₂-cyclohexylene-).

The term “cycloalkenylene,” as used herein by itself or as part of another group, refers to a substituted alicyclic divalent hydrocarbon radical having from 3 to 15 carbon atoms, preferably 3 to 10, and at least one carbon-carbon double bond. Examples of “cycloalkenylene” as used herein include, but are not limited to, 4,5-cyclopentene-1,3-diyl, 3,4-cyclohexene-1,1-diyl, and the like. Cycloalkenylene additionally refers to a divalent hydrocarbon radical as defined for cycloalkylene and having at least one single bond replaced with a double bond. The double bond may be contained in the ring structure. Alternatively, when possible, the double bond may be located on an acyclic portion of the cycloalkeneylene moiety.

The term “cycloheteroalkylene,” as used herein by itself or as part of another group, refers to a cycloalkylene group as described above, wherein 1 to 5 of the carbon atoms indicated is replaced by a heteroatom chosen from N, O, or S. In one embodiment, the oxygen, nitrogen, and sulfur atoms contained therein do not form bonds with other heteroatoms. Suitable examples include the diradicals of piperidine, piperazine, morpholine, and pyrrolidine. Other suitable examples include methylenepiperidyl, ethylenepiperidyl, methylenepiperazinyl, ethylenepiperazinyl, and methylenemorpholinyl.

The term “cycloheteroalkenylene,” as used herein by itself or as part of another group, refers to a cycloalkenylene group as described above, wherein 1 to 5 of the carbon atoms indicated is replaced by a heteroatom chosen from N, O, or S. In one embodiment, the oxygen, nitrogen, and sulfur atoms contained therein do not form bonds with other heteroatoms.

The term “alkoxy,” as used herein by itself or as part of another group, refers to any of the above alkyl groups linked to an oxygen atom. Typical examples are methoxy, ethoxy, isopropyloxy, sec- butyloxy, and t-butyloxy.

The term “alkenyloxy,” as used herein by itself or as part of another group, refers to any of the above alkenyl groups linked to an oxygen atom. Typical examples include ethenyloxy, propenyloxy, butenyloxy, pentenyloxy, and hexenyloxy.

The term “aryl,” as used herein by itself or as part of another group, refers to monocyclic or bicyclic aromatic groups containing from 6 to 14 carbons in the ring portion, preferably 6-10 carbons in the ring portion.

Typical examples include phenyl, naphthyl, anthracenyl, or fluorenyl.

The term “aralkyl” or “arylalkyl,” as employed herein by itself or as part of another group, refers to C₁₋₆ alkyl groups as defined above having an aryl substituent, such as benzyl, phenylethyl, or 2-naphthylmethyl.

The term “heteroaryl,” as used herein by itself or as part of another group, refers to groups having 5 to 14 ring atoms; 6, 10, or 14 π electrons shared in a cyclic array; and containing carbon atoms and 1, 2, 3, or 4 oxygen, nitrogen, or sulfur atoms. Examples of heteroaryl groups are: thienyl, benzo[b]thienyl, naphtho[2,3-b]thienyl, thianthrenyl, furyl, pyranyl, isobenzofuranyl, benzoxazolyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, 4αH-carbazolyl, carbazolyl, β-carbolinyl, phenanthridinyl, acridinyl, perimidinyl, phenanthrolinyl, phenazinyl, isothiazolyl, phenothiazinyl, isoxazolyl, furazanyl, phenoxazinyl, and tetrazolyl groups. Further heteroaryls are described in A. R. Katritzky and C. W. Rees, eds., Comprehensive Heterocyclic Chemistry: The Structure, Reactions, Synthesis and Use of Heterocyclic Compounds, Vol. 1-8, Pergamon Press, NY (1984).

The term “alkylenedioxy,” as used herein by itself or as part of another group, refers for a ring and is especially C₁₋₄ alkylenedioxy. Alkylenedioxy groups may optionally be substituted with halogen (especially fluorine).

Typical examples include methylenedioxy (—OCH₂O—) or difluoromethylenedioxy (—OCF₂O—).

The term “halogen” or “halo,” as used herein by itself or as part of another group, refers to chlorine, bromine, fluorine or iodine.

The term “monoalkylamine” or “monoalkylamino,” as used herein by itself or as part of another group, refers to the group NH₂ wherein one hydrogen has been replaced by an alkyl group, as defined above.

The term “dialkylamine” or “dialkylamino,” as used herein by itself or as part of another group refers to the group, NH₂ wherein both hydrogens have been replaced by alkyl groups, as defined above.

The term “hydroxyalkyl,” as used herein by itself or as part of another group, refers to any of the above alkyl groups wherein one or more hydrogens thereof are substituted by one or more hydroxyl moieties.

The term “acylamino,” as used herein refers to a moiety of the formula —NR^{a}C(O)R^{b}, wherein R^{a} and R^{b} are independently hydrogen or alkyl groups is defined above.

The term “haloalkyl,” as used herein by itself or as part of another group, refers to any of the above alkyl groups wherein one or more hydrogens thereof are substituted by one or more halo moieties. Typical examples include fluoromethyl, trifluoromethyl, trichloroethyl, and trifluoroethyl.

The term “haloalkenyl,” as used herein by itself or as part of another group, refers to any of the above alkenyl groups wherein one or more hydrogens thereof are substituted by one or more halo moieties. Typical examples include fluoroethenyl, difluoroethenyl, and trichloroethenyl.

The term “carboxyalkyl,” as used herein by itself or as part of another group, refers to any of the above alkyl groups wherein one or more hydrogens thereof are substituted by one or more carboxylic acid moieties.

The term “heteroatom” is used herein to mean an oxygen atom (“O”), a sulfur atom (“S”) or a nitrogen atom (“N”). It will be recognized that when the heteroatom is nitrogen, it may form an NR^{a}R^{b} moiety, wherein R^{a} and R^{b} are, independently from one another, hydrogen or alkyl, or together with the nitrogen to which they are bound, form a saturated or unsaturated 5-, 6-, or 7-membered ring.

The term “oxy” means an oxygen (O) atom.

The term “thio” means a sulfur (S) atom.

Generally and unless defined otherwise, the phrase “optionally substituted” used herein refers to a group or groups being optionally substituted with one or more substituents independently selected from the group consisting of amino, hydroxy, nitro, halogen, cyano, thiol, C₁₋₆ alkyl, C₂₋₆ alkenyl, C₂₋₆ alkynyl, C₃₋₆ cycloalkyl, C₃₋₆ cycloalkenyl, C₃₋₆ cycloheteralkyl, C₃₋₆ cycloheteroalkenyl, C₆₋₁₀ aryl, 5-10 membered heteroaryl, C₁₋₆ alkoxy, C₃₋₆ alkenyloxy, C₁₋₆ alkylthio, C₁₋₆ alkylenedioxy, C₁₋₆ alkoxy(C₁₋₆)alkyl, C₆₋₁₀ aryl(C₁₋₆)alkyl, C₆₋₁₀ aryl(C₂₋₆)alkenyl, C₆₋₁₀ aryl(C₁₋₆)alkoxy, C₁₋₆ aminoalkyl, C₁₋₆ aminoalkoxy, C₁₋₆ hydroxyalkyl, C₂₋₆ hydroxyalkoxy, benzamido, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, mono(C₁₋₄)alkylamino(C₂₋₆)alkoxy, di(C₁₋₄)alkylamino(C₂₋₆)alkoxy C₂₋₁₀ mono(carboxyalkyl)amino, bis(C₂₋₁₀ carboxyalkyl)amino, aminocarbonyl, C₆₋₁₄ aryl(C₁₋₆) alkoxycarbonyl, C₂₋₆ alkynylcarbonyl, C₁₋₆ alkylsulfonyl, C₂₋₆ alkynylsulfonyl, C₆₋₁₀ arylsulfonyl, C₆₋₁₀ aryl(C₁₋₆)alkylsulfonyl, C₁₋₆ alkylsulfinyl, C₁₋₆ alkylsulfonamido, C₆₋₁₀ arylsulfonamido, C₆₋₁₀ aryl(C₁₋₆) alkylsulfonamido, C₁₋₆ alkyliminoamino, formyliminoamino, C₂₋₆ carboxyalkoxy, C₂₋₆ carboxyalkyl, and carboxy(C₁₋₆)alkylamino.

When the phrase “optionally substituted” is used with reference to an alkyl, alkenyl, or alkynyl group, the phrase “optionally substituted” herein refers to said group or groups being optionally substituted with one or more substituents independently selected from the group consisting of amino, hydroxy, nitro, halogen, cyano, thiol, C₃₋₆ cycloalkyl, C₃₋₆ cycloalkenyl, C₃₋₆ cycloheteralkyl, C₃₋₆ cycloheteroalkenyl, C₆₋₁₀ aryl, 5-10 membered heteroaryl, C₁₋₆ alkoxy, C₃₋₆ alkenyloxy, C₁₋₆ alkylthio, C₁₋₆ alkylenedioxy, C₁₋₆ alkoxy(C₁₋₆) alkyl, C₆₋₁₀ aryl(C₁₋₆)alkyl, C₆₋₁₀ aryl(C₂₋₆)alkenyl, C₆₋₁₀ aryl(C₁₋₆)alkoxy, C₁₋₆ aminoalkyl, C₁₋₆ aminoalkoxy, C₁₋₆ hydroxyalkyl, C₂₋₆ hydroxyalkoxy, benzamido, mono(C₁₋₄) alkylamino, di(C₁₋₄)alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, mono(C₁₋₄)alkylamino(C₂₋₆)alkoxy, di(C₁₋₄)alkylamino(C₂₋₆)alkoxy C₂₋₁₀ mono(carboxyalkyl)amino, bis(C₂₋₁₀ carboxyalkyl) amino, C₆₋₁₄ aryl(C₁₋₆)alkoxycarbonyl, C₂₋₆ alkynylcarbonyl, C₁₋₆ alkylsulfonyl, C₂₋₆ alkynylsulfonyl, C₆₋₁₀ arylsulfonyl, C₆₋₁₀ aryl(C₁₋₆)alkylsulfonyl, C₁₋₆ alkylsulfinyl, C₁₋₆ alkylsulfonamido, C₆₋₁₀ arylsulfonamido, C₆₋₁₀ aryl(C₁₋₆) alkylsulfonamido, C₁₋₆ alkyliminoamino, formyliminoamino, C₂₋₆ carboxyalkoxy, C₂₋₆ carboxyalkyl, and carboxy(C₁₋₆)alkylamino.

Although detailed definitions have not been provided for every term used above, each term is understood by one of ordinary skill in the art.

As defined above in certain embodiments, the linkers L¹ and L² may be a linker containing 1-10 carbon and/or heteroatoms and which is optionally substituted. This is understood to mean that the linkers may contain any combination of carbon atoms and heteroatoms, such that the sum of number of carbon and heteroatoms, excluding any optional substituents, equals an integer from 1 to 10. Thus, in accordance with the invention, suitable linkers may include, but not necessarily limited to: a linker containing 1 carbon atom (e.g., CH₂); a linker containing one heteroatom (e.g., O); a linker containing five carbon atoms (e.g., CH₂CH₂CH₂CH₂CH₂); a linker containing 3 carbon atoms and 2 heteroatoms (e.g., OCH₂CH₂NHCH₂); a linker containing 10 carbon atoms; or a linker containing nine carbon atoms and 1 heteroatom.

As mentioned above, the above described compounds may be used to inhibit a taste modulating protein. Such inhibition may be in vitro or in vivo. The amount of the compound of Formula I, or any of the specific subgroups, subclasses, or specific compounds described above, used to inhibit the taste modulating protein may not necessarily be the same when used in vivo compared to in vitro. Factors such as pharmacokinetics and pharmacodynamics of the particular compound may require that a larger or smaller amount of the compound of Formula I, or any of the specific subgroups, subclasses, or specific compounds described above, be used when inhibiting a taste modulating protein in vivo. Accordingly, one aspect of the present invention is a method of inhibiting a taste modulating protein, comprising contacting the taste modulating protein with a compound according to Formula I, or any of the specific subgroups, subclasses, or specific compounds described above. In one embodiment of this aspect of the present invention, the method comprises contacting a cell with a compound of Formula I, or any of the specific subgroups, subclasses, or specific compounds described above, wherein said cell expresses said taste modulating protein. In another embodiment of the present invention, the method comprises administering a compound of Formula I, or any of the specific subgroups, subclasses, or specific compounds described above, to a subject in an amount sufficient to inhibit a taste modulating protein, wherein said subject has or expresses said taste modulating protein. Furthermore, when administered orally, the compound may be dispersed or diluted by saliva.

By way of example, the present invention is directed to a method of inhibiting a taste modulating protein, comprising contacting said protein with a compound of Formula I, or any of the specific subclasses and specific compounds listed above, and inhibiting the protein by at least about 10%, 20%, 30%, 40%, 50%, 60%, 70%, 80%, 90%, or 95%, or from about 50% to about 99%. In another embodiment, the method comprises contacting said protein with a compound of Formula I, or any of the specific subclasses and specific compounds listed above, and inhibiting the protein by about 10% to about 50%. In another embodiment, the present invention is directed to a method of inhibiting a taste modulating protein, comprising contacting said protein with a compound of Formula I, or any of the specific subclasses and specific compounds listed above, and inhibiting the protein by at least about 10%, 20%, 30%, 40%, 50%, 60%, 70%, 80%, 90%, or 95%, or from about 50% to about 99%, or alternatively from about 10% to about 50%, and wherein said taste modulating protein is a naturally occurring taste modulating protein. In another embodiment, the present invention is directed to a method of inhibiting a taste modulating protein, comprising contacting said protein with a compound of Formula I, or any of the specific subclasses or specific compounds listed above, and inhibiting the protein by at least about 10%, 20%, 30%, 40%, 50%, 60%, 70%, 80%, 90%, or 95%, or from about 50% to about 99%, or alternatively from about 10% to about 50%, and wherein said protein is a naturally occurring human taste modulating protein.

Any amount of the compound of Formula I that provides the desired degree of inhibition can be used. For example, a compound of Formula I may be used at a concentration of about 0.1 μM to about 1,000, μM to inhibit a taste modulating protein. Alternatively, concentrations of about 1, 10 or 100 μM of a compound of Formula I may be used to inhibit a taste modulating protein. In certain embodiments, a single dose or two to four divided daily doses, provided on a basis of about 0.001 to 100 mg per kilogram of body weight per day, preferably about 0.01 to about 25 mg/kg of body weight per day is appropriate. The substance is preferably administered orally, but parenteral routes such as the subcutaneous, intramuscular, intravenous or intraperitoneal routes or any other suitable delivery system, such as intranasal or transdermal routes can also be employed.

As used herein, the term “inhibiting” and grammatical variants thereof refers to interfering with the normal activity of. For example, inhibiting a taste modulating protein means interfering with the normal activity of a taste modulating protein. Inhibiting includes but is not necessarily limited to modulating, modifying, inactivating, and the like.

As used herein, the phrase “taste modulating protein” refers to a TRPM5 protein, and includes naturally and recombinantly produced TRPM5 proteins; natural, synthetic, and recombinant biologically active polypeptide fragments of said protein; biologically active polypeptide variants of said protein or fragments thereof, including hybrid fusion proteins and dimers; biologically active polypeptide analogs of said protein or fragments or variants thereof, including cysteine substituted analogs. The taste modulating protein may be a nonhuman protein, for example a nonhuman mammalian protein, or in other embodiments a nonhuman protein such as but not limited to a cow, horse, sheep, pig, chicken, turkey, quail, cat, dog, mouse, rat, rabbit, monkey, or guinea pig taste modulating protein. The taste modulating protein may be generated and/or isolated by any means known in the art. An example of the taste modulating protein and methods of producing the protein are disclosed in, for example, Liu and Liman, Proc. Nat'l Acad. Sci. USA 100: 15160-15165 (2003); D. Prawitt, et al., Proc. Nat'l Acad. Sci. USA 100:15166-71 (2003); and Ulrich, N. D., et al., Cell Calcium 37: 267-2

(2005); each of which is fully incorporated by reference herein.

A homologue is a protein that may include one or more amino acid substitutions, deletions, or additions, either from natural mutations of human manipulation. Thus, by way of example, a taste modulating protein may include one or more amino acid substitutions, deletions or additions, either from natural mutations or human manipulation. As indicated, changes are preferably of a minor nature, such as conservative amino acid substitutions that do not significantly affect the folding or activity of the protein.

The variant taste modulating proteins which may be inhibited in accordance with the present invention comprise non-conservative modifications (e.g., substitutions). By “nonconservative” modification herein is meant a modification in which the wild-type residue and the mutant residue differ significantly in one or more physical properties, including hydrophobicity, charge, size, and shape. For example, modifications from a polar residue to a nonpolar residue or vice-versa, modifications from positively charged residues to negatively charged residues or vice versa, and modifications from large residues to small residues or vice versa are nonconservative modifications. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine. In one embodiment, the variant taste modulating proteins used in accordance with the present invention have at least one nonconservative modification.

In other embodiments, the method of the invention comprises inhibiting a taste modulating protein that is a nonhuman protein, such as but not limited to a cow, horse, sheep, pig, chicken, turkey, quail, cat, dog, mouse, rat, rabbit, monkey, or guinea pig taste modulating protein.

An additional aspect of the present invention is a method of inhibiting the depolarization of a taste receptor cell, comprising contacting the taste receptor cell with a compound according to Formula I, or any of the specific subgroups, subclasses, or specific compounds described above. For example, a compound of Formula I may inhibit the depolarization of a taste receptor cell be a mechanism other than, or in addition to, the mechanism of inhibiting a taste receptor protein. In one embodiment of this aspect of the present invention, the method comprises contacting a taste receptor cell with a compound of Formula I, or any of the specific subgroups, subclasses, or specific compounds described above, wherein said taste receptor cell can detect a sweet, bitter, sour, salty, or umami taste. In another embodiment of the present invention, the method comprises administering a compound of Formula I, or any of the specific subgroups, subclasses, or specific compounds described above, to a subject in an amount sufficient to inhibit the depolarization of a taste receptor cell. Furthermore, when administered orally, the compound may be dispersed or diluted by saliva.

By way of example, the present invention is directed to a method of inhibiting the depolarization of a taste receptor cell, comprising contacting said taste receptor cell with a compound of Formula I, or any of the specific subclasses and specific compounds listed above, and inhibiting the depolarization of the taste receptor cell by at least about 10%, 20%, 30%, 40%, 50%, 60%, 70%, 80%, 90%, or 95%, or from about 60% to about 99%, or alternatively from about 30% to about 75%. In another embodiment, the present invention is directed to a method of inhibiting the depolarization of a taste receptor cell, comprising contacting said protein with a compound of Formula I, or any of the specific subclasses and specific compounds listed above, and inhibiting the depolarization of the taste receptor cell by at least about 10%, 20%, 30%, 40%, 50%, 60%, 70%, 80%, 90%, or 95%, or from about 50% to about 99%, or alternatively from about 20% to about 60%, and wherein said taste receptor cell is a naturally occurring taste modulating protein. In another embodiment, the present invention is directed to a method of inhibiting a taste receptor cell, comprising contacting said protein with a compound of Formula I, or any of the specific subclasses or specific compounds listed above, and inhibiting the taste receptor cell by at least about 10%, 20%, 30%, 40%, 50%, 60%, 70%, 80%, 90%, or 95%, or from about 50% to about 99%, or alternatively from about 40% to about 80%, and wherein said taste receptor cell is a human taste receptor cell.

Any amount of the compound of Formula I that provides the desired degree of inhibition can be used. For example, a compound of Formula I may be used at a concentration of about 0.1 μM to about 1,000 μM to inhibit a taste receptor cell. Alternatively, concentrations of about 1 μM, 50 μM, or 100 μM of a compound of Formula I may be used to inhibit the depolarization of a taste receptor cell.

In certain embodiments, a single dose or two to four divided daily doses, provided on a basis of about 0.001 to 100 mg per kilogram of body weight per day, preferably about 0.01 to about 25 mg/kg of body weight per day is appropriate. When inhibiting a taste receptor cell in vivo, the compound of Formula I is preferably administered orally.

In one embodiment of this aspect of the present invention, the method comprises contacting a taste receptor cell with a compound of Formula I, or any of the specific subgroups, subclasses, or specific compounds described above, wherein said taste receptor cell can detect a sweet, bitter, sour, salty, or umami taste. In another embodiment of the present invention, the method comprises administering a compound of Formula I, or any of the specific subgroups, subclasses, or specific compounds described above, to a subject in an amount sufficient to inhibit the depolarization of a taste receptor cell. Furthermore, when administered orally, the compound may be dispersed or diluted by saliva.

In another embodiment, a compound according to Formula I, or any of the specific subgroups, subclasses, or specific compounds described above, is useful for inhibiting a taste, such as an undesirable taste of a food product. Examples of food products having an undesirable taste include, but are not necessarily limited to, citrus fruits such as grapefruit, orange, and lemon; vegetables such as tomato, pimento, celery, melon, carrot, potato and asparagus; seasoning or flavoring materials, such as soy sauce and red pepper; soybean products; fish products; meats and processed meats; dairy products such as cheese; breads and cakes; and confectioneries such as candies, chewing gum and chocolate. Other examples of food products envisioned in accordance with the present invention are described below and throughout the specification.

The method may be performed such that the taste of the food product being inhibited by the compound of Formula I is inhibited by at least about 10%, 20%, 30%, 40%, 50%, 60%, 70%, 80%, 90%, or 95%, or from about 60% to about 99%, or alternatively from about 20% to about 50%. Thus, in a more specific embodiment, the method comprises administering a food product comprising one or more food ingredients and one or more compounds according to Formula I, wherein the one or more compounds according to Formula I are present in an amount sufficient to inhibit a bitter taste, produced by the food product, by at least about 10%, 20%, 30%, 40%, 50%, 60%, 70%, 80%, 90%, or 95%, or from about 60% to about 99%, or alternatively from about 30% to about 70%. Of course, in other embodiments, a taste may be inhibited to differing extents.

Any amount of the compound of Formula I that provides the desired degree of taste inhibiting can be used. For example, a compound of Formula I may be used at a concentration of about 0.1 μM to about 5,000 μM to inhibit a bitter taste. Alternatively, concentrations of about 1 μM, 100 μM, or 500 μM of a compound of Formula I may be used to inhibit a sweet taste.

A food product may also include beverages and drinks. Examples of drinks having an undesirable or unwanted taste include, but are not limited to, juices of citrus fruits and vegetables, soybean, milk, coffee, cocoa, black tea, green tea, fermented tea, semi-fermented tea, refreshing drinks, beverages and milk. In certain embodiments, the taste inhibiting effective amount of a compound according to Formula I, or any of the specific subgroups, subclasses, or specific compounds described above, has a range of from about 0.01 to about 5.0 grams per 100 mL. In other embodiments, the taste inhibiting effective amount of a compound according to Formula I, or any of the specific subgroups, subclasses, or specific compounds described above, has a range of from about 0.5 to about 2 grams per 100 mL. Alternatively, a compound according to Formula I, or any of the specific subgroups, subclasses, or specific compounds described above, is administered in an amount of about 1 gram per 100 mL.

The method of the present invention in its various embodiments may be used to inhibit one or more tastes selected from the group consisting of sweet, bitter, sour, salty, or umami. Preferably, the method of the present invention inhibits a bitter and/or sweet taste.

As used herein, the phrase “inhibit a taste” and grammatical variants thereof, such as “taste inhibiting” and “inhibiting a taste,” refers to interfering with the perception of a taste. The taste may be sensed to a lesser degree or not sensed at all by application of the present invention.

An additional aspect of the present invention is a method of inhibiting a taste of a pharmaceutical composition, comprising administering a compound according to Formula I, or any of the specific subgroups, subclasses, or specific compounds described above, to a subject receiving the pharmaceutical composition. The compound of Formula I may be administered together with the pharmaceutical composition as separate compositions, for example either concurrently or sequentially. The compound of Formula I may administered, or caused to be administered, prior to the pharmaceutical agent producing the taste to be inhibited. Alternatively, the compound for Formula I may be administered as a component of the pharmaceutical composition.

By way of example, the method may be performed such that the taste being inhibited by the compound of Formula I is inhibited by at least about 10%, 20%, 30%, 40%, 50%, 60%, 70%, 80%, 90%, or 95%, or from about 60% to about 99%, or alternatively from about 25% to about 50%. Thus, in a more specific embodiment, the method comprises administering a pharmaceutical composition comprising a pharmaceutically active agent, optionally one or more excipients, and one or more compounds according to Formula I, wherein the one or more compounds according to Formula I are present in an amount sufficient to inhibit a bitter taste, produced by the pharmaceutically active agent, by at least about 10%, 20%, 30%, 40%, 50%, 60%, 70%, 80%, 90%, or 95%, or from about 60% to about 99%, or alternatively from about 30% to about 60%. In another embodiment, the compound of Formula I is administered in a ratio of from about 10:1 to about 1:10 in relation to the pharmaceutical agent.

By way of additional examples, the method of inhibiting a taste of a pharmaceutical composition may comprise inhibiting a taste produced by one or more agents selected from the group consisting of antipyretics, analgesics, laxatives, appetite depressants, antacidics, antiasthmatics, antidiuretics, agents active against flatulence, antimigraine agents, psychopharmacological agents, spasmolytics, sedatives, antihyperkinetics, tranquilizers, antihistaminics, decongestants, beta-receptor blockers, agents for alcohol withdrawal, antitussives, fluorine supplements, local antibiotics, corticosteroid supplements, agents against goiter formation, antiepileptics, agents against dehydration, antiseptics, NSAIDs, gastrointestinal active agents, alkaloids, supplements for trace elements, ion-exchange resins, cholesterol-depressant agents, lipid-lowering agents, antiarrhythmics, and expectorants. Further specific examples of pharmaceutical compostions in accordance with the method of the invention are described below.

Additionally, the method of inhibiting a taste of a pharmaceutical composition may comprise inhibiting a taste produced by a counterterrorism pharmaceutical. Because of the increased risk of terrorist attacks, such as chemical, nuclear, or biological attacks, the use of counterterrorism pharmaceutical agents is expected to increase in the future. A counterterrorism pharmaceutical agent includes those pharmaceutical agents that are useful in counteracting agents that can be used in a terrorist attack. Agents that have been used in terrorist acts, or considered as useful for carrying out future terrorist acts, include ricin, sarin, radioactive agents and materials, and anthrax. Pharmaceutical agents that counteract these agents are useful as a counterterrorism pharmaceutical. Such counterterrorism pharmaceuticals include, but are not limited to, antiobiotics such as ciprofloxacin and doxycycline; potassium iodide; and antiviral agents. Thus, in one embodiment of the present invention, the method may be performed such that the taste of a counterterrorism pharmaceutical, such as an antiobiotic such as ciprofloxacin and doxycycline; potassium iodide; or an antiviral agent, is inhibited by the compound of Formula I by at least about 10%, 20%, 30%, 40%, 50%, 60%, 70%, 80%, 90%, or 95%, or from about 60% to about 99%, or alternatively from about 25% to about 50%. In another embodiment, the compound of Formula I is administered in a ratio of from about 10:1 to about 1:10 in relation to the counterterrorism agent.

In another embodiment, a compound according to Formula I, or any of the specific subgroups, subclasses, or specific compounds described above, is useful for inhibiting an undesirable taste of a nutriceutical composition. Examples of nutriceutical compositions having an undesirable taste include, but are not necessarily limited to, enteral nutrition products for treatment of nutritional deficit, trauma, surgery, Crohn's disease, renal disease, hypertension, obesity and the like, to promote athletic performance, muscle enhancement or general well being or inborn errors of metabolism such as phenylketonuria. In particular, such nutriceutical formulations may contain one or more amino acids which have a bitter or metallic taste or aftertaste. Such amino acids include, but are not limited to, an essential amino acids selected from the group consisting of L isomers of leucine, isoleucine, histidine, lysine, methionine, phenylalanine, threonine, tryptophan, tyrosine, and valine. Further specific examples of nutraceutical compostions in accordance with the method of the invention are described below.

By way of example, the method may be performed such that the taste being inhibited by the compound of Formula I is inhibited by at least about 10%, 20%, 30%, 40%, 50%, 60%, 70%, 80%, 90%, or 95%, or from about 60% to about 99%, or alternatively from about 20% to about 50% Thus, in a more specific embodiment, the method comprises administering a nutraceutical composition comprising a nutraceutical agent, optionally one or more excipients, and one or more compounds according to Formula I, wherein the one or more compounds according to Formula I are present in an amount sufficient to inhibit a undesired taste, produced by the nutraceutical agent, by at least about 10%, 20%, 30%, 40%, 50%, 60%, 70%, 80%, 90%, or 95%, or from about 60% to about 99%, or alternatively from about 10% to about 50%.

A compound according to Formula I may be incorporated into medical and/or dental compositions. Certain compositions used in diagnostic procedures have an unpleasant taste, such as contrast materials and local oral anesthetics. The inhibitors of the invention may be used to improve the comfort of subjects undergoing such procedures by improving the taste of compositions. In addition, the inhibitors of the invention may be incorporated into pharmaceutical compositions, including tablets and liquids, to improve their flavor and improve patient compliance particularly where the patient is a child or a non-human animal).

In another embodiment, a compound according to Formula I, or any of the specific subgroups, subclasses, or specific compounds described above, is used to inhibit a taste of a cosmetic product. For example, but not by way of limitation, a compound according to Formula I may be incorporated into face creams, lipsticks, lipgloss, and the like. Also, a compound according to Formula I, or any of the specific subgroups, subclasses, or specific compounds described above, can be used to inhibit an unpleasant taste of lipbalm, such as Chapstick® or Burt's Beeswax® Lip Balm.

In addition, a compound according to Formula I, or any of the specific subgroups, subclasses, or specific compounds described above, may be incorporated into compositions that are not traditional foods, pharmaceuticals, or cosmetics, but which may contact taste membranes. Examples include, but are not limited to, soaps, shampoos, toothpaste, denture adhesive, and glue on the surfaces of stamps and envelopes. Thus, the present invention also covers a process of preparing a composition that is not a traditional food, pharmaceutical, or cosmetic, but which may contact taste membranes, according to conventional methods, wherein the improvement comprises adding a compound of Formula I to said composition.

In another embodiment, a compound according to Formula I, or any of the specific subgroups, subclasses, or specific compounds described above, is used to inhibit a bitter taste associated with one or more the following: bitter pharmaceutical alkaloids such as acetaminophen, ampicillin, chlorpheniramine, chlarithromycin, doxylamine, guaifenesin, ibuprofen, pseudoephidrine hydrochloride, and ranitidine, bitter pharmaceutical metallic salts such as zinc containing bioadhesives (denture adhesive), bitter vitamins, bitter components of foods such as creatine, limonin, naringin, quinizolate, and bitter components of beverages such as caffeine, and humulone. In one embodiment, the concentration of the compound according to Formula I used is in the range of 0.01 mM to 20 mM and may vary depending on the amount of bitter compound used and its bitterness.

In another embodiment, the present invention is directed to a method of inhibiting the taste of a veterinary product, such as veterinary medicines, veterinary food products, veterinary supplements, and the like, that are administered to domesticated animals. In a preferred embodiment, a compound according to Formula I, or any of the specific subgroups, subclasses, or specific compounds described above, is used to inhibit a taste of a veterinary product administered to a cat or dog.

In one embodiment, in each of the methods of inhibiting a taste described herein, a compound according to Formula I, or any of the specific subgroups, subclasses, or specific compounds described above, is administered in an amount effective to inhibit said taste. As a nonlimiting example, the taste inhibiting effective amount of a compound according to Formula I, or any of the specific subgroups, subclasses, or specific compounds described above, administered in one embodiment is from about 0.01 to about 5.0 grams per 100 mL.

In other embodiments, in the taste inhibiting methods described herein, a compound according to Formula I, or any of the specific subgroups, subclasses, or specific compounds described above, is administered in an amount that is sufficient, in combination with the administration of one or more additional taste inhibiting agents, to inhibit said taste. For example, in a method of inhibiting the bitter taste of a liquid pharmaceutical composition, the composition comprises a compound according to Formula I and another taste inhibiting agent, wherein the amount of the compound of Formula I is about 25% to about 75% of the amount required to inhibit the bitter taste in the absence of the other taste inhibiting agent.

In another embodiment, the present invention is directed to a method of decreasing the palatability and/or intake of food, comprising administering to a subject in need of such treatment one or more compounds according to Formula I, or any of the specific subgroups, subclasses, or specific compounds described above, in an amount sufficient to decrease the palatability and/or intake of food. Taste modulating protein knockout mice have been shown to have diminished taste preference for sucrose, artificial sweeteners, and umami flavors and diminished taste aversion to bitter solutions. See Zhang et al., Cell 112:293-301 (2003). Thus, according to the present invention, a compound according to Formula I, or any of the specific subgroups, subclasses, or specific compounds described above, may be administered to a subject so that the palatability of food, as experienced by said subject, is decreased. Without being bound by theory, it is believed that a lower palatability of food can lead to a lower intake of food by the subject. Thus, in certain embodiments, by administering a compound according to Formula I, or any of the specific subgroups, subclasses, or specific compounds described above, to a subject, the subject will consume a decreased amount of food compared to the subject's food intake when not being administered a compound of Formula I, or any of the specific subgroups, subclasses, or specific compounds described above. In other embodiments, by administering a compound according to Formula I, or any of the specific subgroups, subclasses, or specific compounds described above, to a subject, the subject will have a lower caloric intake compared to the subject's caloric intake when not being administered a compound of Formula I, or any of the specific subgroups, subclasses, or specific compounds described above. In other embodiments, administering a compound according to Formula I, or any of the specific subgroups, subclasses, or specific compounds described above, to a subject can be a dieting means to facilitate or aid weight loss.

In each of the embodiments of methods described above, the subject of the method, unless otherwise limited to, may be any animal which is need of the particular treatment or effect of the method. Such animals include but are not limited to a cow, horse, sheep, pig, chicken, turkey, quail, cat, dog, mouse, rat, rabbit, monkey, or guinea pig taste modulating protein. In other embodiments, the animal is a livestock animal, a domesticated animal, or an animal kept as a pet. In particular embodiments, the subject of the claimed method is a human.

Furthermore, in each of the embodiments of the methods described herein, a compound of Formula I may be used in varying ratios to the agent that is believed to cause the unwanted taste, such as a bitter or sweet taste. For example, a compound of Formula I may be administered in a molar ratio of about 1000:1 to about 1:1000, or alternatively administered in a molar ratio of about 500:1, about 200:1, about 10:1, about 1:1, about 1:10, about 1:200, or about 1:500, relative to the agent that is believed to cause the unwanted taste. In another example, the present invention is directed to a method of inhibiting a bitter taste of a pharmaceutical composition, comprising administering to a subject in need of such method a pharmaceutical composition and a compound according to Formula I, wherein the pharmaceutical composition comprises a pharmaceutically active agent and optionally one or more excipients, and wherein the compound according to Formula I is administered as either a component of the pharmaceutical composition or as a separate dosage form, and wherein molar ratio of the compound of Formula I to the pharmaceutically active agent about 1000:1 to about 1:1000, or alternatively administered in a molar ratio of about 500:1, about 200:1, about 10:1, about 1:1, about 1:10, about 1:200, or about 1:500. As will be appreciated, the various ranges and amounts of the compound of Formula I can be used, with modifications if preferred, in each of the embodiments described herein.

Compositions

The present invention is also directed to various, useful compositions comprising a compound of Formula I or a physiologically acceptable salt thereof.

In one aspect, the present invention is directed to a pharmaceutical composition comprising a compound of Formula I, as defined above, including any of the specific embodiments, subclasses, or species described above, and one or more pharmaceutically acceptable carriers. Preferred compositions of the present invention are pharmaceutical compositions comprising a compound selected from one or more embodiments listed above, and one or more pharmaceutically acceptable excipients. Pharmaceutical compositions that comprise one or more compounds of Formula I, or any of the specific subgroups, subclasses, or specific compounds described above, may be used to formulate pharmaceutical drugs containing one or more active agents that exert a biological effect other than taste inhibition and/or inhibition of a taste modulating protein.

The pharmaceutical composition preferably further comprises one or more active agents that exert a biological effect. Such active agents includes pharmaceutical and biological agents that have an activity other than taste inhibition. Such active agents are well known in the art. See, e.g., The Physician's Desk Reference. Such compositions can be prepared according to procedures known in the art, for example, as described in Remington's Pharmaceutical Sciences, Mack Publishing Co., Easton, Pa., USA. In one embodiment, such an active agent includes bronchodilators, anorexiants, antihistamines, nutritional supplements, laxatives, analgesics, anesthetics, antacids, H₂-receptor antagonists, anticholinergics, antidiarrheals, demulcents, antitussives, antinauseants, antimicrobials, antibacterials, antifungals, antivirals, expectorants, anti-inflammatory agents, antipyretics, and mixtures thereof. The pharmaceutical composition according to the present invention may comprise one or more compounds according to Formula I, as described above, or any of the specific subgroups, subclasses, or specific compounds described above; an active agent that has a bitter taste; and optionally one or more pharmaceutically acceptable carriers.

In another embodiment, the active agent is selected from the group consisting of antipyretics and analgesics, e.g., ibuprofen, acetaminophen, or aspirin; laxatives, e.g., phenolphthalein dioctyl sodium sulfosuccinate; appetite depressants, e.g., amphetamines, phenylpropanolamine, phenylpropanolamine hydrochloride, or caffeine; antacidics, e.g., calcium carbonate; antiasthmatics, e.g., theophylline; antidiuretics, e.g., diphenoxylate hydrochloride; agents active against flatulence, e.g., simethecon; migraine agents, e.g., ergotaminetartrate; psychopharmacological agents, e.g., haloperidol; spasmolytics or sedatives, e.g., phenobarbitol; antihyperkinetics, e.g., methyldopa or methylphenidate; tranquilizers, e.g., benzodiazepines, hydroxinmeprobramates or phenothiazines; antihistaminics, e.g., astemizol, chloropheniramine maleate, pyridamine maleate, doxlamine succinate, bromopheniramine maleate, phenyltoloxamine citrate, chlorocyclizine hydrochloride, pheniramine maleate, and phenindamine tartrate; decongestants, e.g., phenylpropanolamine hydrochloride, phenylephrine hydrochloride, pseudoephidrine hydrochloride, pseudoephidrine sulfate, phenylpropanolamine bitartrate, and ephedrine; beta-receptor blockers, e.g., propanolol; agents for alcohol withdrawal, e.g., disulfiram; antitussives, e.g., benzocaine, dextromethorphan, dextromethorphan hydrobromide, noscapine, carbetapentane citrate, and chlophedianol hydrochloride; fluorine supplements, e.g., sodium fluoride; local antibiotics, e.g., tetracycline or cleocine; corticosteroid supplements, e.g., prednisone or prednisolone; agents against goiter formation, e.g., colchicine or allopurinol; antiepileptics, e.g., phenytoine sodium; agents against dehydration, e.g., electrolyte supplements; antiseptics, e.g., cetylpyridinium chloride; NSAIDs, e.g., acetaminophen, ibuprofen, naproxen, or salts thereof; gastrointestinal active agents, e.g., loperamide and famotidine; various alkaloids, e.g., codeine phosphate, codeine sulfate, or morphine; supplements for trace elements, e.g., sodium chloride, zinc chloride, calcium carbonate, magnesium oxide, and other alkali metal salts and alkali earth metal salts; vitamins; ion-exchange resins, e.g., cholestyramine; cholesterol-depressant and lipid-lowering substances; antiarrhythmics, e.g., N-acetylprocainamide; and expectorants, e.g., guaifenesin.

Active substances which have a particularly unpleasant taste include antibacterial agents such as ciprofloxacin, ofloxacin, and pefloxacin; antiepileptics such as zonisamide; macrolide antibiotics such as erythromycin; beta-lactam antibiotics such as penicillins and cephalosporins; psychotropic active substances such as chlorpromazine; active substances such as sulpyrine; and agents active against ulcers, such as cimetidine.

In another embodiment, the pharmaceutical composition comprises one or more compounds according to Formula I, or any of the specific subgroups, subclasses, or specific compounds described above, and at least one amino acid selected from the group consisting of glycine, L-alanine, L-arginine, L-aspartic acid, L-cystine, L-glutamic acid, L-glutamine, L-histidine, L-isoleucine, L-leucine, L-lysine, L-methionine, L-ornithine, L-phenylalanine, L-proline, L-serine, L-threonine, L-tryptophan, L-tyrosine, L-valine, creatine, and mixtures thereof.

In another embodiment, the pharmaceutical composition comprises one or more compounds according to Formula I, or any of the specific subgroups, subclasses, or specific compounds described above; a biologically active agent that exhibits an activity other than taste inhibition; and at least one amino acid, such as one selected from the group consisting of glycine, L-alanine, L-arginine, L-aspartic acid, L-cystine, L-glutamic acid, L-glutamine, L-histidine, L-isoleucine, L-leucine, L-lysine, L-methionine, L-ornithine, L-phenylalanine, L-proline, L-serine, L-threonine, L-tryptophan, L-tyrosine, L-valine, creatine, and mixtures thereof.

The pharmaceutical compositions of the present invention can be in any form suitable to achieve their intended purpose. Preferably, however, the composition is one which can be administered buccally or orally. Alternatively, the pharmaceutical composition may be an oral or nasal spray.

The pharmaceutical compositions of the invention can be in any form suitable for administration to any animal that can experience the beneficial effects of one or more compounds according to Formula I, or any of the specific subgroups, subclasses, or specific compounds described above. Foremost among such animals are humans, although the invention is not intended to be so limited. Other suitable animals include canines, felines, dogs, cats, livestock, horses, cattle, sheep, and the like. A veterinary composition, as used herein, refers to a pharmaceutical composition that suitable for non-human animals. Such veterinary compositions are known in the art.

The pharmaceutical preparations of the present invention can be manufactured using known methods, for example, by means of conventional mixing, granulating, dragée-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Pharmaceutical excipients are well known in the art. Suitable excipients include fillers such as saccharides, for example, lactose or sucrose, mannitol or sorbitol, cellulose preparations and/or calcium phosphates, for example, tricalcium phosphate or calcium hydrogen phosphate, as well as binders, such as, starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents can be added, such as, the above-mentioned starches and also carboxymethyl-starch, cross-linked polyvinyl pyrrolidone, agar, or alginic acid or a salt thereof, such as, sodium alginate. Auxiliaries are, above all, flow-regulating agents and lubricants, for example, silica, talc, stearic acid or salts thereof, such as, magnesium stearate or calcium stearate, and/or polyethylene glycol. Dragée cores are provided with suitable coatings that, if desired, are resistant to gastric juices. For this purpose, concentrated saccharide solutions can be used, which may optionally contain gum arabic, talc, polyvinyl pyrrolidone, polyethylene glycol, and/or titanium dioxide, lacquer solutions and suitable organic solvents or solvent mixtures. In order to produce coatings resistant to gastric juices, solutions of suitable cellulose preparations, such as, acetylcellulose phthalate or hydroxypropylmethyl-cellulose phthalate, are used. Dye stuffs or pigments can be added to the tablets or dragee coatings, for example, for identification or in order to characterize combinations of active compound doses.

Liquid dosage forms for oral administration include pharmaceutically acceptable emulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

In a further embodiment, the invention is directed to a chewable tablet comprising one or more compounds according to Formula I and one or more biologically active agents. Chewable tablets are known in the art. See, e.g., U.S. Pat. Nos. 4,684,534 and 6,060,078 each of which is incorporated by reference in its entirety. Any kind of medicament may be contained in the chewable tablet, preferably a medicament of bitter taste, natural plant extracts or other organic compounds. More preferably, vitamins such as vitamin A, vitamin B, vitamin B₁, vitamin B₂, vitamin B₆, vitamin C, vitamin E and vitamin K; natural plant extracts such as Sohgunjung-tang extracts, Sipchundaebo-tang extracts and Eleutherococcus senticosus extracts; organic compounds such as dimenhydrinate, meclazine, acetaminophen, aspirin, phenylpropanolamine, and cetylpyridinium chloride; or gastrointestinal agents such as dried aluminum hydroxide gel, domperidone, soluble azulene, L-glutamine and hydrotalcite may be contained in the core.

In another embodiment, the present invention is directed to an orally disintegrating composition wherein said orally disintegrating composition further comprises one or more compounds according to Formula I, or any of the specific subgroups, subclasses, or specific compounds described above. Orally disintegrating tablets are known in the art. See, e.g., U.S. Pat. Nos. 6,368,625 and 6,316,029, each of which is hereby incorporated by reference in its entirety.

In another embodiment, the present invention is further directed to a nasal composition further comprising one or more compounds according to Formula I, or any of the specific subgroups, subclasses, or specific compounds described above. Nasal sprays are known in the art. See, e.g., U.S. Pat. No. 6,187,332. Addition of one or more compounds according to Formula I to a nasal spray can reduce the experience of an unpleasant taste associated with the composition of the nasal spray. By way of a nonlimiting example, a nasal spray composition according to the present invention comprises water (such as 95-98 weight percent), a citrate (such as 0.02 M citrate anion to 0.06 M citrate anion), a compound according to Formula I, and optionally phosphate (such as 0.03 M phosphate to 0.09 M phosphate).

In another embodiment, the present invention is directed to a solid dosage form comprising a water and/or saliva activated effervescent granule, such as one having a controllable rate of effervescence, and a compound according to Formula I, or any of the specific subgroups, subclasses, or specific compounds described above. The effervescent composition may further comprise a pharmaceutically active compound. Effervescent pharmaceutical compositions are known in the art. See, e.g., U.S. Pat. No. 6,649,186, which is incorporated by reference in its entirety. The effervescent composition can be used in pharmaceutical, veterinary, horticultural, household, food, culinary, pesticidal, agricultural, cosmetic, herbicidal, industrial, cleansing, confectionery and flavoring applications. Formulations incorporating the effervescent composition comprising a compound according to Formula I can further include one or more additional adjuvants and/or active ingredients which can be chosen from those known in the art including flavors, diluents, colors, binders, filler, surfactant, disintegrant, stabilizer, compaction vehicles, and non-effervescent disintegrants.

In another embodiment, the present invention is directed to a film-shaped or wafer-shaped pharmaceutical composition that comprises a compound -according to Formula I, or any of the specific subgroups, subclasses, or specific compounds described above, and is capable of disintegrating. Such a film-shaped or wafer-shaped pharmaceutical composition can be configured, for example, as quickly disintegrating administration forms, e.g., administration forms disintegrating within a period of 1 second up to 3 minutes, or as slowly disintegrating administration forms, e.g., administration forms disintegrating within a period of 3 to 15 minutes.

The indicated disintegration times can be set to the above-mentioned ranges by using, for example, matrix-forming polymers which have different disintegrating, or solubility, characteristics. Thus, by mixing the corresponding polymer components, the disintegration time can be adjusted. In addition, disintegrants are known which “draw” water into the matrix and cause the matrix to burst open from within. As a consequence, certain embodiments of the invention include such disintegrants for the purpose of adjusting the disintegration time.

Suitable are polymers for use in the film-shaped or wafer-shaped pharmaceutical composition include cellulose derivatives, polyvinyl alcohol (e.g. MOWIOL™), polyacrylates, polyvinyl pyrrolidone, cellulose ethers, such as ethyl cellulose, as well as polyvinyl alcohol, polyurethane, polymethacrylates, polymethyl methacrylates and derivatives and copolymerisates of the aforementioned polymers.

In certain embodiments, the total thickness of the film-shaped or wafer-shaped pharmaceutical composition according to the invention is preferably 5 μm up to 10 μm, preferably 30 μm to 2 mm, and with particular preference 0.1 mm to 1 mm. The pharmaceutical preparations may round, oval, elliptic, triangular, quadrangular or polygonal shape, but they may also have any rounded shape.

In another embodiment, the present invention is directed to a composition comprising a medicament or agent contained in a coating that surrounds a gum base formulation and further comprising a taste-inhibiting amount of a compound according to Formula I, or any of the specific subgroups, subclasses, or specific compounds described above. Preferably, the coating comprises at least 50% by weight of the entire product. As the center is chewed, the medicament or agent is released into the saliva. For example, U.S. Pat. No. 6,773,716, which is incorporated herein by reference in its entirety, discloses a suitable medicament or agent contained in a coating that surrounds a gum base formulation. One or more compounds according to Formula I, or any of the specific subgroups, subclasses, or specific compounds described above, can be used in preparing the coating. Optionally, the composition may further comprise high-intensity sweeteners and appropriate flavors. It has been found that with respect to certain medicaments or agents that may have an astringent or bitter taste that by adding a inhibiting agent to the formulation, that a much more palatable formulation, including the medicament, can be provided. In this regard, even though the medicament in, for example, its powder form may be bitter or have an offensive taste, the matrix used as the coating of the present invention, including the inhibiting agent, will afford a product having acceptable medicinal properties. The compound according to Formula I, or any of the specific subgroups, subclasses, or specific compounds described above, may be present in varying amounts, such as about 30% 50%, 75%, or 90%. In another embodiment, the compound according to Formula I may be present in about 30% to about 99%.

In other embodiments, the compound according to Formula I is present in about 1% to about 30%.

In yet another embodiment, the present invention is directed to a process of preparing an improved composition comprising a medicament or agent contained in a coating that surrounds a gum base formulation, wherein the improvement comprises adding a compound according to Formula I, or any of the specific subgroups, subclasses, or specific compounds described above, to the coating that surrounds the gum base formulation. The compound according to Formula I may be added in varying amounts, such as about 30% 50%, 75%, 80%, or 90%, or from about 10% to about 90%. In other embodiments, the compound according to Formula I is present in about 1% to about 30%.

In a further embodiment, the invention is directed to a pharmaceutical composition suitable for aerosol administration, comprising a compound according to Formula I, or any of the specific subgroups, subclasses, or specific compounds described above, and a suitable carrier. The aerosol composition may further comprises pharmaceutically active agent. Aerosol compositions are known in the art. See, e.g., U.S. Pat. No. 5,011,678, which is hereby incorporated by reference in its entirety. As a nonlimiting example, an aerosol composition according to the present invention may comprise a medically effective amount of a pharmaceutically active substance, one or more compounds according to Formula I, or any of the specific subgroups, subclasses, or specific compounds described above, and a biocompatible propellant, such as a (hydro/fluoro)carbon propellant.

In certain embodiments, the pharmaceutical compositions of the invention comprise from about 0.001 mg to about 1000 mg of a compound of Formula I, or any of the specific subgroups, subclasses, or specific compounds described above. In another embodiment, the compositions of the invention comprise from about 0.01 mg to about 10 mg of a compound of Formula I, or any of the specific subgroups, subclasses, or specific compounds described above.

In another embodiment, the composition of the invention comprises a compound of Formula I, or any of the specific subgroups, subclasses, or specific compounds described above, in an amount sufficient to inhibit a taste modulating protein. By way of example, the present invention is pharmaceutical or veterinary composition, comprising a compound of Formula I, or any of the specific subclasses and specific compounds listed above, in an amount sufficient to a taste modulating protein by at least about 10%, 20%, 30%, 40%, 50%, 60%, 70%, 80%, 90%, or 95%, or from about 50% to about 99%, or alternatively from about 10% to about 40%. In another embodiment, the present invention is directed to a method of inhibiting a taste modulating protein, comprising contacting said taste modulating protein with a compound of Formula I, or any of the specific subclasses and specific compounds listed above, and inhibiting the protein by at least about 10%, 20%, 30%, 40%, 50%, 60%, 70%, 80%, 90%, or 95%, or from about 50% to about 99%, or alternatively from about 20% to about 60%, and wherein said taste modulating protein is a naturally occurring taste modulating protein. In another embodiment, the present invention is directed to a method of inhibiting a taste modulating protein, comprising contacting said protein with a compound of Formula I, or any of the specific subclasses and specific compounds listed above, and inhibiting the protein by at least about 10%, 20%, 30%, 40%, 50%, 60%, 70%, 80%, 90%, or 95%, or from about 50% to about 99%, or alternatively from about 20% to about 40%, and wherein said protein is a naturally occurring human taste modulating protein.

In another embodiment, the present invention is directed to a nutriceutical composition comprising one or more nutriceuticals, one or more compounds according to Formula I, or any of the specific subgroups, subclasses, or specific compounds described above, and optionally one or more carriers. Examples of nutriceutical compositions having an undesirable taste include, but are not necessarily limited to, enteral nutrition products for treatment of nutritional deficit, trauma, surgery, Crohn's disease, renal disease, hypertension, obesity and the like, to promote athletic performance, muscle enhancement or general well being or inborn errors of metabolism such as phenylketonuria. In particular, such nutriceutical formulations may contain one or more amino acids which have a bitter or metallic taste or aftertaste. Such amino acids include, but are not limited to, an essential amino acids selected from the group consisting of L isomers of leucine, isoleucine, histidine, lysine, methionine, phenylalanine, threonine, tryptophan, tyrosine, and valine. Additionally, the invention is directed to a process of preparing an improved nutriceutical composition, wherein the improvement comprises adding one or more compounds according to Formula I, or any of the specific subgroups, subclasses, or specific compounds described above, to a nutriceutical composition. In certain embodiments, the one or more compounds according to Formula I, or any of the specific subgroups, subclasses, or specific compounds described above, are added to a nutriceutical composition in an amount of about 1% to about 50% , or about 5%, 10%, or 15%, by weight.

In another embodiment, the present invention is directed to a dental hygienic composition comprising one or more compounds according to Formula I, or any of the specific subgroups, subclasses, or specific compounds described above. Dental hygienic compositions are known in the art and include but are not necessarily limited to toothpaste, mouthwash, plaque rinse, dental floss, dental pain relievers (such as Anbesol™), and the like. For example, the invention includes a dental bleaching composition which comprises one or more compounds of Formula I, or any of the specific subgroups, subclasses, or specific compounds described above, in an amount sufficient to inhibit a bitter taste. Dental bleaching compositions are known in the art. See, e.g., U.S. Pat. No. 6,485,708, which is herein incorporated by reference in its entirety. A dental bleaching composition of the present invention intended for use with dental trays may utilize a sticky carrier formed from a fluid and a thickener. The sticky carrier accordingly may comprise finely divided silica, such as silica fume, dispersed in a liquid, such as a polyol. Examples of suitable polyols include propylene glycol, glycerin, polypropylene glycols, sorbitol, polyethylene glycols and the like. While the carrier preferably includes thickeners, the carrier may also be only a liquid such as water or any of the liquid polyols without any thickeners.

Additionally, the invention is directed to a process of preparing an improved dental hygienic composition, wherein the improvement comprises adding one or more compounds according to Formula I, or any of the specific subgroups, subclasses, or specific compounds described above, to a dental bleaching composition. In certain embodiments, the one or more compounds according to Formula I are added to a dental hygienic composition in an amount of about 1% to about 20%, preferably about 1% to about 5%, or about 5%, 10%, or 15%, by weight.

In another embodiment, the present invention is directed to a cosmetic product comprising one or more compounds according to Formula I, or any of the specific subgroups, subclasses, or specific compounds described above. For example, but not by way of limitation, the cosmetic product comprising a compound according to Formula I, or any of the specific subgroups, subclasses, or specific compounds described above, may be a face cream, lipstick, lipgloss, and the like. Other suitable compositions of the invention include lipbalm, such as Chapstick™ or Burt's Beeswax™ Lip Balm, further comprising one or more compounds according to Formula I, or any of the specific subgroups, subclasses, or specific compounds described above.

Additionally, the invention is directed to a process of preparing an improved cosmetic product, wherein the improvement comprises adding one or more compounds according to Formula I, or any of the specific subgroups, subclasses, or specific compounds described above, to a cosmetic product. In certain embodiments, the one or more compounds according to Formula I, or any of the specific subgroups, subclasses, or specific compounds described above, are added to a cosmetic product in an amount of about 1% to about 20%, preferably about 1% to about 5%, or about 1%, 2%, or 3%, by weight.

In another embodiment, the present invention is directed to a food product comprising one or more compounds according to Formula I, or any of the specific subgroups, subclasses, or specific compounds described above. Preferably, the food product is one which exhibits an undesirable taste, such as a bitter taste, which can be inhibited by a compound according to Formula I, or any of the specific subgroups, subclasses, or specific compounds described above. Furthermore, in a preferred embodiment, the food product comprises a compound of Formula I, or any of the specific subgroups, subclasses, or specific compounds described above in an amount sufficient to inhibit an unpleasant taste.

Specific food products and food ingredients to which one of more compounds of Formula I, or any of the specific subgroups, subclasses, or specific compounds described above, can be added include but are not necessarily limited to, potassium chloride, ammonium chloride, sodium chloride (e.g., table salt), magnesium chloride, halide salts, naringin, caffeine, urea, magnesium sulfate, saccharin, acetosulfames, aspirin, potassium benzoate, potassium bicarbonate, potassium carbonate, potassium nitrate, potassium nitrite, potassium sulfate, potassium sulfite, potassium glutamate, food preservatives in their physiologically acceptable salts, antibiotics, unsweetened chocolate, cocoa beans, yogurt, preservatives, flavor enhancers, dietary supplements, gelling agents, pH control agents, nutrients, processing aids, bodying agents, dispersing agents, stabilizers, colorings, coloring diluents, anticaking agents, antimicrobial agents, formulation aids, leavening agents, surface active agents, anticaking agents, nutrient supplements, alkali, acids, sequestrants, denuding agents, general purpose buffers, thickeners, cooked out juice retention agents, color fixatives in meat and meat products, color fixatives in poultry and poultry products, dough conditioners, maturing agents, yeast foods, mold retardants, emulsifiers, texturizers, binders, water correctives, miscellaneous and general purpose food additives, tableting aids, lye peeling agents, washing water agents, oxidizers, antioxidants, enzymes, extenders, fungicides, cake mixes, coffee, tea, dry mixes, non-dairy creamers, salts, animal glue adjuvant, cheese, nuts, meat and meat products, poultry and poultry product, pork and pork products, fish and fish products, vegetable and vegetable products, fruit and fruit products, smoked products such as meat, cheese fish, poultry, and vegetables, whipping agents, masticatory substances in chewing gums, dough strengtheners, animal feed, poultry feed, fish feed, pork feed, defoaming agents, juices, liquors, substances or drinks containing alcohol, beverages including but not limited to alcoholic beverages and non-alcoholic carbonated and/or non-carbonated soft drinks, whipped toppings, bulking agents used in eatables including but not limited to starches, corn solids, polysaccharides and other polymeric carbohydrates, icings, as well as potassium-containing or metal-containing substances with undesirable tastes and the like.

Moreover, the present invention contemplates the preparation of eatables such as breads, biscuits, pancakes, cakes, pretzels, snack foods, baked goods etc. prepared using for example potassium bicarbonate or potassium carbonate in place of the sodium salts as leavening agents in conjunction with a compound according to Formula I, or any of the specific subgroups, subclasses, or specific compounds described above, in an amount sufficient to eliminate one or more undesirable tastes. The compound according to Formula I, or any of the specific subgroups, subclasses, or specific compounds described above, can be typically present in an amount ranging from about 0.001% to about 50% by weight, preferably about 0.1% to about 10% by weight, or alternatively, from 0.1% to about 1% by weight, of the material with the undesirable taste. The present invention also contemplates the preparation of preservatives for eatables comprising the potassium salts of benzoate, nitrate, nitrite, sulfate, and sulfite and so on, in conjunction with an appropriate concentration of a compound according to Formula I, or any of the specific subgroups, subclasses, or specific compounds described above, to eliminate undesirable tastes in foodstuffs. Thus, the invention is directed to a process of preparing an improved food product, wherein the improvement comprises adding one or more compounds according to Formula I, or any of the specific subgroups, subclasses, or specific compounds described above, to a food product. In certain embodiments, the one or more compounds according to Formula I, or any of the specific subgroups, subclasses, or specific compounds described above, are added to a food product in an amount of about 1% to about 20%, preferably about 1% to about 5%, about 1%, 3%, or 4%, by weight.

In another embodiment, the present invention is directed to an animal food product comprising one or more compounds according to Formula I, or any of the specific subgroups, subclasses, or specific compounds described above. The one or more compounds are preferably in an amount sufficient to inhibit one or more undesirable tastes associated with the animal food product. Animal food products are well known in the art, see, e.g., U.S. Pat. No. 6,403,142, and include dog food, cat food, rabbit food, and the like. The animal food product may also be food products useful for feeding livestock, such as cattle, bison, pigs, chicken, and the like. In another embodiment, the animal food composition of the present invention is a solid hypoallergenic pet food comprising a component that contains protein or protein fragments wherein all of said component is partially hydrolyzed and further comprises one or more compounds according to Formula I, or any of the specific subgroups, subclasses, or specific compounds described above.

Additionally, the invention is directed to a process of preparing an improved animal food product, wherein the improvement comprises adding one or more compounds according to Formula I, or any of the specific subgroups, subclasses, or specific compounds described above, to an animal food product. In certain embodiments, the one or more compounds according to Formula I, or any of the specific subgroups, subclasses, or specific compounds described above, are added to an animal food product in an amount of about 1% to about 25%, about 1% to about 10%, or about 5%, 10%, or 15%, by weight.

In further embodiments of the present invention, any of the compositions described herein and containing a compound according to Formula I may further comprise one or more additional taste masking agents. Such masking agents include but are not limited to the group consisting of sucralose; zinc gluconate; ethyl maltol; glycine; acesulfame-k; aspartame; saccharin; fructose; xylitol; malitol; isomalt; salt; spray dried licorice root; glycyrrhizin; dextrose; sodium gluconate; sucrose; glucono-delta-lactone; ethyl vanillin; and vanillin.

In another embodiment, the present invention is directed to a composition comprising a compound of Formula I, or any of the specific subgroups, subclasses, or specific compounds described above, and a carrier, wherein said carrier is suitable for an assay. Such carriers may include solid carriers and/or liquid carriers. A composition suitable for an assay may, but not necessarily, be sterile. Examples of suitable carriers for assays include dimethylsulfoxide, ethanol, dichloromethane, methanol, and the like. In another embodiment, a composition comprises a compound of Formula I, or any of the specific subgroups, subclasses, or specific compounds described above, and a carrier, wherein the compound is in an amount suitable for inhibiting a taste modulating protein.

In each of the embodiments of the compositions described herein, a compound of Formula I, or any of the specific subgroups, subclasses, or specific compounds described above, may be used in varying ratios to the agent that is believed to cause the unwanted taste, such as a bitter or sweet taste. For example, a composition of the invention may comprise a compound of Formula I in a molar ratio of about 1000:1 to about 1 :1000, or alternatively administered in a molar ratio of about 500:1, about 200:1, about 10:1, about 1:1, about 1:10, about 1:200, or about 1:500, relative to the agent that is believed to cause the unwanted taste, such as a bitter or sweet taste. In another example, the present invention is directed to a food product comprising one or more food ingredients and a compound according to Formula I, wherein the molar ratio of the compound of Formula I to the food agent that causes, or is believed to cause, a bitter taste about 1000:1 to about 1:1000, or alternatively administered in a molar ratio of about 500:1, about 200:1, about 10:1, about 1:1, about 1:10, about 1:200, or about 1:500. As will be appreciated, the various ranges and amounts of the compound of Formula I can be used, with modifications if preferred, in each of the embodiments described herein.

The activity of a compound according to Formula I, or any of the specific subgroups, subclasses, or specific compounds described above can be determined by testing said compound using a number of methods known in the art. For example, one can evaluate the ability of a compound to inhibit a bitter taste by using an in vivo taste assay. This in vivo assay identifies the bitter blockers that by testing their activity using human subjects. A concentration of the bitter compound quinine in water is found that the subject rates as 5 for bitterness on a scale of 0 to 10, where 0 is no bitterness and 10 is the most intense bitterness the subject has ever encountered. This concentration of quinine is then made up containing a concentration of a compound according to Formula I to be tested, and the subject rates the bitterness of this solution on the same scale.

The activity of a compound according to Formula I, or any of the specific subgroups, subclasses, or specific compounds described above, can also be determined by means of the assay described in Example 23. The assay is described in complete detail in copending application Ser. No. ______(Attorney Docket No. 2305.0170001), filed Nov. 3, 2006, which is incorporated by reference herein in its entirety.

Compounds

An additional aspect of the present invention is directed to novel compounds according to Formula I. Novel compounds according to Formula I are useful in the methods and compositions as described herein. The various embodiments of the compounds include any and all of the specific genera, subgenera, subgroups, and individual compounds described herein.

In a further embodiment, the invention is directed to a compound according to the following formula

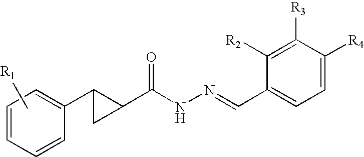

wherein R¹ is hydrogen or halogen; R² is hydrogen or C₁₋₄ haloalkyl; R³ is hydrogen, C₁₋₄ haloalkyl, C₁₋₄ alkoxy, or C₁₋₄ alkylthio; and R⁴ is hydrogen, C₁₋₄ haloalkyl, C₁₋₄ alkoxy, or C₁₋₄ alkylthio. In another embodiment, R¹ is hydrogen or halogen; R² is CF₃; R³ is hydrogen, C₁₋₄ haloalkyl, C₁₋₄ alkoxy, or C₁₋₄ alkylthio; and R⁴ is hydrogen, C₁₋₄ haloalkyl, C₁₋₄ alkoxy, or C₁₋₄ alkylthio. Suitable alkoxy groups include methoxy. Suitable haloalkyl groups include trifluoromethoxy. Suitable alkylthio groups include —SCH₃. Preferably, the compounds are trans-cyclopropyl comopunds. Examples of compounds of the present invention are described herein, for example in the Examples.

Methods of Preparation of Compounds

A compound according to Formula I can be synthesized according to methods outlined in the following descriptions. The compounds for use in the present invention can be synthesized using procedures known in the art.

The following general schemes illustrate synthetic methods used to prepare compounds of the present invention. In one process, a compound of Formula I can be prepared by condensing a suitable acylated hydrazide with a suitable ketone or aldehyde in a suitable organic solvent, such as ethanol, 2-propanol, tetrahydrofuran, toluene, etc., and mixtures thereof, as shown in Scheme 1 (wherein R¹, R², R³, R⁴, L¹, and L² are defined as above). The presence of a water quenching agent such as molecular sieves or dry potassium carbonate may be useful in the process. An acid or a base catalysis may be used to facilitate the condensation. Acid catalysts include, but are not limited to, p-toluenesulfonic acid, methylsulfonic acid, phosphoric acid, and sulfuric acid. Base catalysts include, but are not limited to, triethylamine, diisopropylethylamine, pyridine, N-methylmorpholine, sodium carbonate, potassium carbonate, and sodium carbonate.

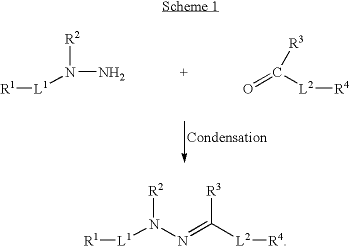

In an alternative process, certain compounds according to Formula I, wherein R² is H, can be prepared as shown in Scheme 2 (wherein R¹, R², R³, R⁴, L¹, and L²are defined as above). According to this process, a suitable carboxylic acid is treated with a hydrazone of a suitable aldehyde or ketone to provide a compound according to Formula I. Carbonyldiimidazole and triethylamine can be employed as condensing agents in this reaction, although other suitable condensing agents may be used as well.

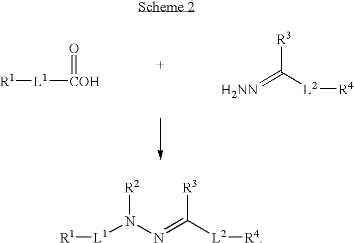

As a further example, the compounds of Formula I, wherein R¹ and R² are aryl groups, can be prepared by condensing an acylated hydrazide (such as compound 1) with an aldehyde (such as compound 2) in a suitable organic solvent, such as ethanol, 2-propanol, tetrahydrofuran, toluene, etc., and mixtures thereof, and in the presence of a water quenching agent such as molecular sieves or dry potassium carbonate (Scheme 1). An acid or a base catalysis may be used to facilitate the condensation. Acid catalysts include, but are not limited to, p-toluenesulfonic acid, methylsulfonic acid, phosphoric acid, and sulfuric acid. Base catalysts include, but are not limited to, triethylamine, diisopropylethylamine, pyridine, N-methylmorpholine, sodium carbonate, potassium carbonate, and sodium carbonate. An example of this process is shown in Scheme 3.

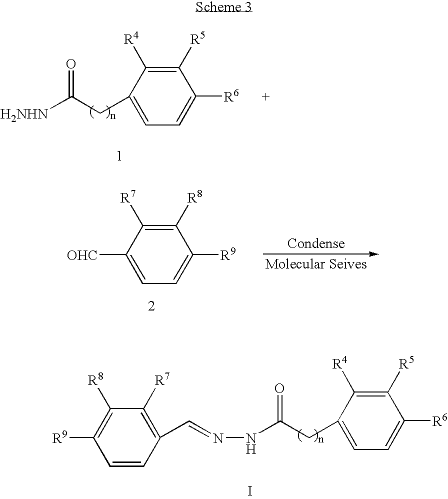

The variation of this method would include treating a suitable carboxylic acid (such as compound 3) with a hydrazone of a suitable aldehyde (such as compound 4) to provide compound I. The carbonyldiimidazole and triethylamine are usually employed as condensing agents in this reaction. An example of this process is shown in Scheme 4.

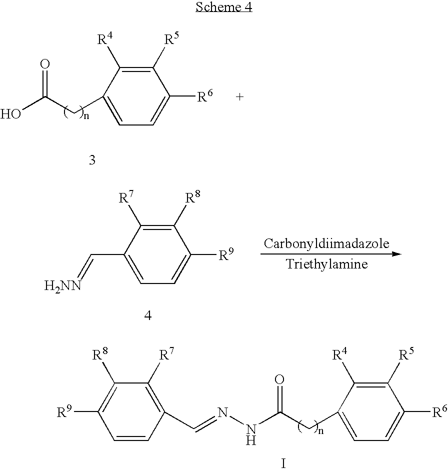

The reaction can also be carried out neat (e.g., without a solvent). After the reaction is complete, the product can be isolated by crystallization from solvents such as ethanol, dichloromethane, ethyl acetate, and toluene etc.

Similarly other compounds of this invention can be obtained from commercial sources and prepared by those skilled in the art. Starting materials are commercially available or they can be prepared by ordinary persons trained in the art. For example, compound 1 shown above can be prepared by reacting a carboxylic acid (such as compound 3) with a protected hydrazine (such as compound 5) in the presence of carbonyldiimidazole/triethyl amine to provide a protected acid hydrazide (such as compound 6). After the reaction is complete, the protecting group from the acid hydrazide (such as compound 6) can be removed under standard conditions (such as acidic conditions, e.g., trifluoroacetic acid) to provide a compound of formula 1. An example of this process is shown in Scheme 5.

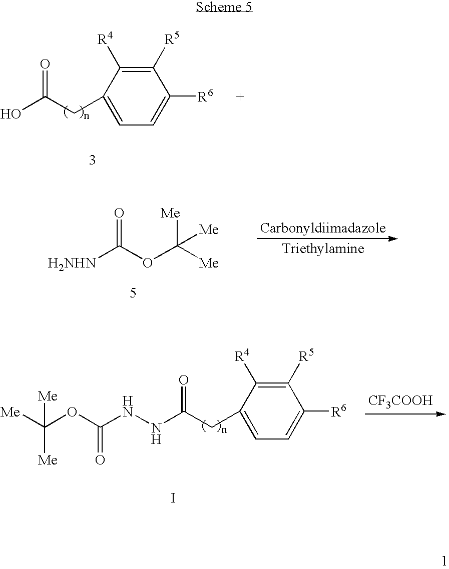

Other compounds of this invention can be prepared by slight variation of the methods described herein. These methods and others are described in the literature, such as Wyrzykiewicz and Prukala, Polish J. Chem. 72:694-702 (1998); Elderfield and Wood, J. Org. Chem. 27:2463-2465 (1962), each of which is incorporated by reference in its entirety.

Of course, other methods and procedures known in the art may be used to prepare certain compounds of Formula I.

The following examples are illustrative, but not limiting, of the method, compounds, and compositions of the present invention. Each of the compounds listed below was obtained from commercially available catalog companies, such as Aldrich RarechemLib, Aldrich Sigma, AlsInEx, Biotech Corp., Brandon/Berlex, Calbiochem, ChemBridge, Comgenex West, Foks H, G. & J. Research, IBS, ICN Biochemicals, Institute for Chemotherapy, Kodak, Lederle Labs, Ligand-CGX, Maybridge PRI, Menai Organics, Menai/Neurocrine, MicroSource, MPA Chemists, Mybrgd/ONYX, PRI-Peakdale, RADIAN, Receptor Research, RGI, Rhone-Poulenc, SPECS/BioSPECS/SYNTHESIA, T. Glinka, Tripos Modern, VWR, Zaleska, Zelinksy/Berlex, Aeros, and Chemica. The compounds were purified using conventional purificiation procedures, such as HPLC. The identity of the compound was confirmed using HPLC and mass spectrometry. Analytical LC-MS was performed on a 75×4.6 mm Atlantis DC₁₈ column using a solvent system of Buffer A (100% water with 0.1% formic acid) and Buffer B (100% acetonitrile). At a flow rate of 1.0 mL/min, 1.5 mL of 70% Buffer B was passed over the column, followed by a 1.5 mL linear gradient to 95% Buffer B, followed by an isocratic wash with 1.5 mL of 95% Buffer B. As is known in the art and noted above, the hydrazone moiety can exist in either the E or the Z conformation. Thus, while a particular stereochemistry may be indicated for particular compounds described herein, it is understood that the invention includes all stereoisomers, and in particular all E and Z isomers. Other suitable modifications and adaptations of the variety of conditions and parameters normally encountered and obvious to those skilled in the art are within the spirit and scope of the invention.

### EXAMPLES

### Example 1

### Methyl 4-((E)-((Z)-1-(2-(benzo[d]thiazol-2-yl)hydrazono) -2-methylpropyl)diazenyl)benzoate

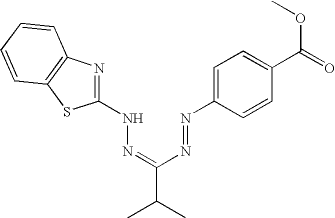

Molecular Formula: C₁₉H₁₉N₅O₂S; Molecular Weight: 381.5 (calculated).

### Example 2

### (E)-2-(4-Bromo-2-((2-(quinolin -8-yl)hydrazono)methyl)phenoxy)acetic acid

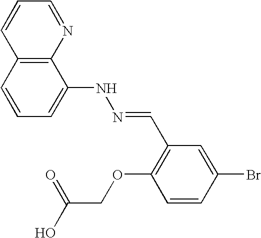

Molecular Formula: C₁₈H₁₄BrN₃O₃; Molecular Weight: 400 (calculated).

### Example 3

### (E)-N′-(3,4-Dimethoxybenzylidene)-2-(naphthalene-1-yl)acetohydrazide

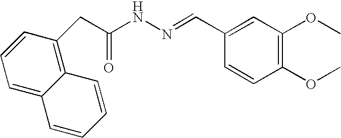

Molecular Formula: C₂₁H₂₀N₂O₃; Molecular Weight: 348 (calculated), 348 (found).

### Example 4

### (E)-N′-(3,4-Dimethoxybenzylidene)-2-phenylcyclopropanecarbohydrazide

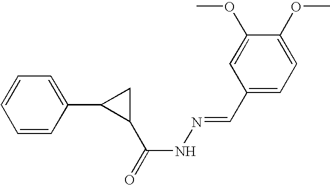

Molecular Formula: C₁₉H₂₀N₂O₃; Molecular Weight: 324 (calculated), 324 (found).

### Example 5

### (E)-3-Cyclohexenyl-4-hydroxy-N′-(4-methoxybenzylidene)butanehydrazide

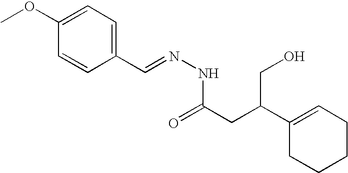

Molecular Formula: C₁₈H₂₄N₂O₃; Molecular Weight 316.40 (calculated).

### Example 6

### (E)-N′-(3,4-Dimethoxybenzylidene)-4-hydroxyhexanehydrazide

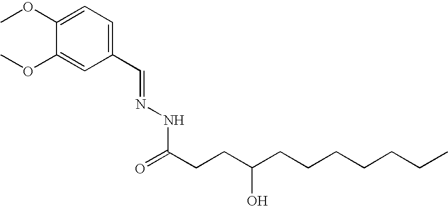

Molecular Formula: C₂₀H₃₀N₂O₄; Molecular Weight: 364.5 (calculated), 364 (found).

### Example 7

### 2-((Z)-2-(Phenyl-((E)-phenyldiazenyl) -methylene)hydrazinyl)benzoic acid

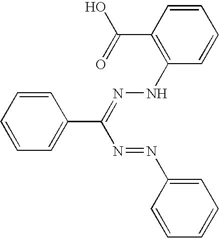

Molecular Formula: C₂₀H₁₆N₄O₂; Molecular Weight: 344.7 (calculated).

### Example 8

### (E)-N′-(3,4-Dimethoxybenzylidene)-2-(m-tolyloxy)acetohydrazide

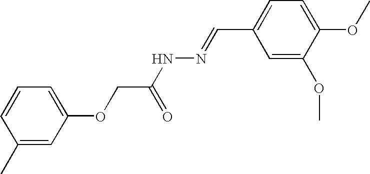

Molecular Formula: C₁₈H₂₀N₂O₄; Molecular Weight: 328 (calculated), 328 (found).

### EXAMPLE 9

### (E)-N′-(4-(Allyloxy)-3-methoxybenzylidene)-2-(3-bromobenzylthio)acetohydrazide

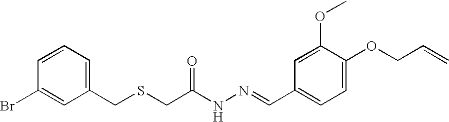

Molecular Formula: C₂₀H₂₁BrN₂O₃S; Molecular Weight: 449 (calculated), 447.9 (found).

### Example 10

### (E)-N′-(4-Isopropylbenzylidene)bicyclo[4.1.0.]heptane-7-carbohydrazide

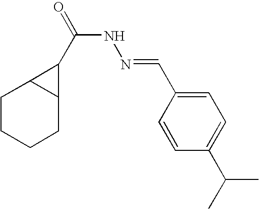

Molecular Formula: C₁₈H₂₄N₂O; Molecular Weight: 284 (calculated), 284 (found).

### Example 11

### (Z)-1,3,3-Trimethyl-2-((E)-2-(2-(4-nitrophenyl)hydrazono)ethylidene)indoline

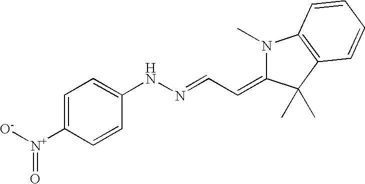

Molecular Formula: C₁₉H₂₀N₄O₂; Molecular Weight: 336 (calculated), 336 (found).

### Example 12

### (E)-N′-(4-(Diethylamino)-2-hydroxybenzylidene) -2-phenylcyclopropanecarbohydrazide

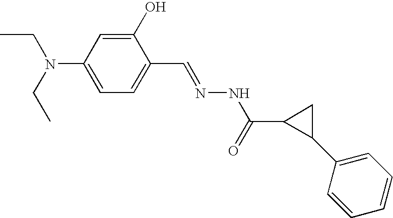

Molecular Formula: C₂₁H₂₅N₃O₂; Molecular Weight: 351 (calculated), 351 (found).

### Example 13

### (4-(Trifluoromethylthio)penyl)carbonohydrazonoyldicyanide

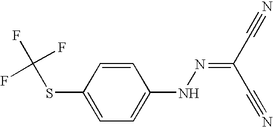

Molecular Formula: C₁₀H₅F₃N₄S; Molecular Weight: 270.24 (calculated).

### Example 14

### N-((E)-3-((Z)-2-(1,5-Dimethyl-2-oxoindolin-3-ylidene)hydrazinyl)-3-oxo-1-phenylprop-1-en-2-yl)benzamide

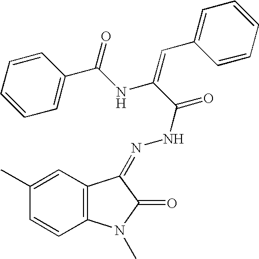

Molecular Formula: C₂₆H₂₂N₄O₃; Molecular Weight: 438.5 (cal'd).

### Example 15

### (Z)-2-(2-((1-Butyl-1H-indol-3-yl)methylene)hydrazinyl)benzoic acid

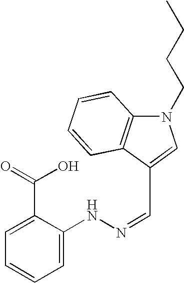

Molecular Formula: C₂₀H₂₁N₃O₂; Molecular Weight: 335.4 (calculated).

### Example 16

### (E)-4-((2-Benzyl-2-phenylhydrazono)methyl)pyridine

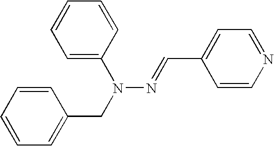

Molecular Formula: C₁₉H₁₇N₃; Molecular Weight: 287 (calculated), 287.2 (found).

### Example 17

### (Z)-N′-((1H-Pyrrol-2-yl)methylene)tricyclo [3.3.1^{3,7}]decane-3-carbohydrazide

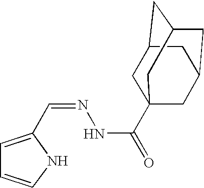

Molecular Formula: C₁₆H₁₂N₃O; Molecular Weight: 271 (calculated).

### Example 18

### (Z)-1-(2-(4-(Ethyl-(2-hydroxyethyl)-amino)phenyl)hydrazono)naphthalen-2(1H)-one

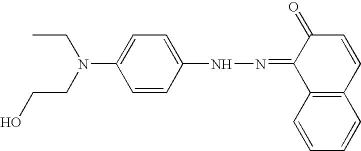

Molecular Formula: C₂₀H₂₁N₃O₂; Molecular Weight: 335 (calculated), 333.2 (found).

### Example 19

### (E)-4-((2-(5-Chloro-3-(trifluoromethyl)pyridini-2-yl)-2-2-methylhydrazono)methyl)benzene-1,3-diol

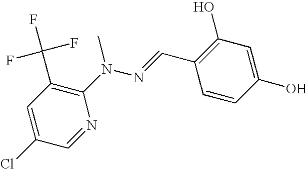

Molecular Formula: C₁₄H₁₁ClF₃N₃O; Molecular Weight: 345.7 (calculated), 344.9 (found).

### Example 20

### (E)-2-(3,4-Dimethylphenylamino)-N′-(4-morpholino-3-nitrobenzylidene)acetohydrazide

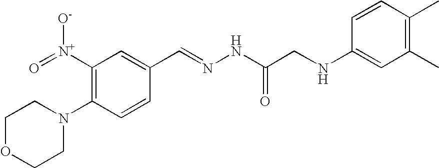

Molecular Formula: C₂₁H₂₅N₅O₄; Molecular Weight: 411.4 (calculated), 411.3 (found).

### Example 21

### (Z)-3-(2-Nitro-5-(pyrrolidin-1-yl)phenyl)hydrazono)quinuclidine

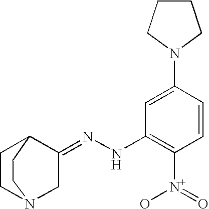

Molecular Formula: C₁₇H₂₃N₅O₂; Molecular Weight: 329.4 (calculated).

### Example 22

### (E)-2-((2-(1H-Benzo[d]imidazol-2-yl)hydrazono)methyl)-5-(diethylamino)phenol

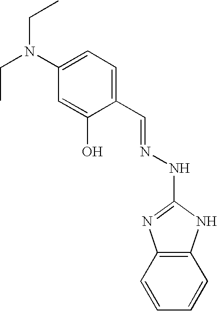

Molecular Formula: C₁₈H₂₁N₅O; Molecular Weight: 323.4 (calculated).

### Example 23

### N-(3-(2-((6-Bromobenzo[d][1,3]dioxol-5-yl)methylene)-hydrazinyl)-1-(4-(dimethylamino)phenyl)-3-oxoprop-1-en-2-yl)benzamide

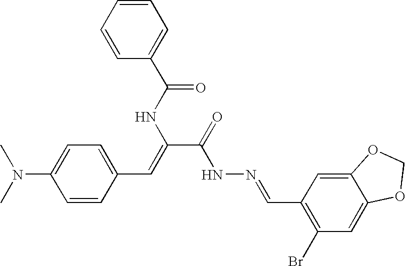

Molecular Formula: C₂₆H₂₃BrN₄O₄; Molecular Weight: 535.4 (calc'd)

### Example 24

### N-(1-(4-(Diethylamino)phenyl)-3-(2-(4-hydroxy-3-iodo-5-methoxybenzylidene)hydrazinyl)-3-oxoprop-1-en-2-yl)benzamide

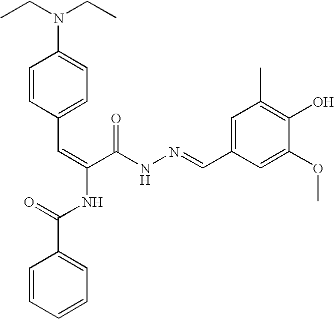

Molecular Formula: C₂₈H₂₉IN₄O₄; Molecular Weight: 612.5 (calculated)

### Example 25

### N′-(4-Hydroxy-3-methoxybenzylidene)-3-(1-hydroxy-cyclopentyl)propanehydrazide

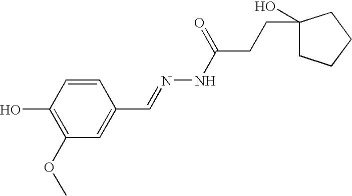

Molecular Formula: C₁₆H₂₂N₂O₄; Molecular Weight: 306.4 (calculated)

### Example 26

### 4-Nitro-N′-(3,4,5-trimethoxybenzylidene)benzohydrazide

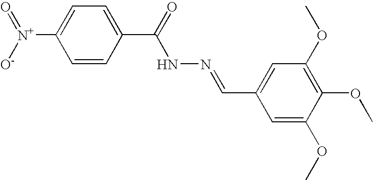

Molecular Formula: C₁₇H₁₇N₃O₆; Molecular Weight: 359.3 calculated)

### Example 27

### N′-(4-(diethylamino)-2-hydroxybenylidine) -phenylcyclopropanecarboxhydrazide

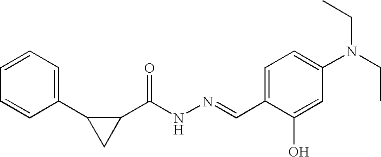

Molecular Formula: C₂₁H₂₅N₃O₂; Molecular Weight: 351.4 calculated)

### Example 28

### N′-(5-Bromo-2-oxoindolin-3-ylidene)-2-(2-bromo-4-methoxyphenoxy)acetohydrazide

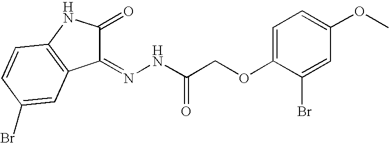

Molecular Formula: C₁₇H₁₃Br₂N₃O₄; Molecular Weight: 483.1 (calculated)

### Example 29

### 3-(1H-indol-3-yl)-N′-(3,4,5-trimethoxybenzylidene)propanehydrazide

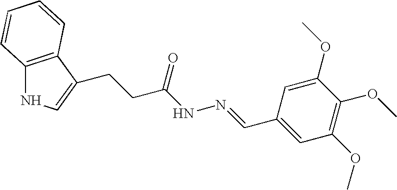

Molecular Formula: C₂₁H₂₃N₃O₄; Molecular Weight: 381.4 (calculated)

### Example 30

### N′-(2-oxoindolin-3-ylidene)-2-(2-methyl-4-(1,1-dimethylethyl)phenoxy)acetohydrazide

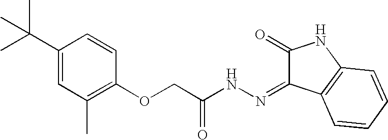

Molecular Formula: C₂₁H₂₃N₃O₃; Molecular Weight: 365.4 (calculated)

### Example 31

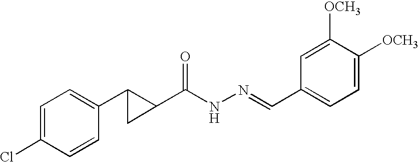

A mixture of 4-chlorobenzaldehyde (10 g, 71 mmol), malonic acid (8.1 g, 78 mmol), piperidine (0.70 mL), and pyridine (60 mL) was heated to reflux for 4 hours. The reaction mixture was cooled to 0° C. and acidified with 6 N hydrochloric acid to form a precipitate. The precipitate was collected by filtration and dried to provide 4-chlorocinnamic acid.

Thionyl chloride (12.4 mL, 0.167 mmol) was added dropwise over a 20 minute period to a 0° C. solution of a portion of the preceding solid (12.2 g, 66.8 mmol) in methanol (130 mL). The solution was then heated at 80° C. for 20 hours. The solution was cooled to room temperature and the volatiles were removed in vacuo. The residue was taken up in ethyl acetate (200 mL). The mixture was washed (3×100 mL with saturated sodium bicarbonate, 2×200 mL with water, 1×100 mL with saturated sodium chloride), dried (sodium sulfate) and concentrated in vacuo to provide methyl 4-chlorocinnamate.

A portion of the preceding product (5.0 g, 25.4 mmol) was dissolved in dichloromethane (50 mL). The solution was protected from light, palladium acetate was added and the mixture was cooled to −30° C. Ethereal diazomethane (prepared from 21.0 g of N-methyl-N-nitrosourea) was added dropwise to the stirred mixture. The excess diazomethane was quenched with acetic acid and the mixture was concentrated in vacuo. The residue was taken up in dichloromethane. The resultant mixture was washed (2×60 mL with saturated sodium bicarbonate, 2×60 mL with water, 1×60 mL with saturated sodium chloride), dried (sodium sulfate) and concentrated in vacuo. The residue was chromatographed (silica, ethyl acetete/hexanes) to provide methyl 2-(4-chlorophenyl)cyclopropane carboxylate.

Hydrazine hydrate (1.45 g, 29 mmol) was added to a stirred solution of a portion of the preceding product (5.1 g, 24 mmol) in methanol (50 mL). After stirring overnight, the reaction mixture was diluted with water and concentrated to remove methanol. The resultant mixture was extracted with ethyl acetate. The organic layers were washed with water (50 mL) and saturated sodium chloride (50 mL), dried (sodium sulfate) and concentrated in vacuo. The product was triturated with ether (4×) and was then dried to provide 2-(4-chlorophenyl)cyclopropane carboxhydrazide.

A solution of 2-(4-chlorophenyl)cyclopropane carboxhydrazide (50 mg, 0.24 mmol) in ethanol (5 mL) was stirred for 10 min. Acetic acid (4 drops) was added to the solution. After stirring for 3 hours, the solvent was removed in vacuo. The product was purified by trituration to provide 2-(4-chlorophenyl)-N′-(3,4-dimethoxybenzylidene)cyclopropanecarboxhydrazide: LCMS m/z 359/361, t_{R}=1.39 min.

### Examples 32-66

The following examples were prepared using the method described in Example 31.

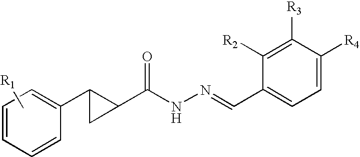

| | | | | | LC-MS(t_{R} | |
|---|---|---|---|---|---|---|
| Example | R₁ | R₂ | R₃ | R₄ | (min), m/z | Name |
| 32 | 2-Cl | — | OMe | OMe | 1.31, | 2-(2-chlorophenyl)-N′-(3,4- |
| | | | | | 359/361 | dimethoxybenylidine)cyclopropane- |
| | | | | | | carboxhydrazide |
| 33 | 3-Cl | — | OMe | OMe | 1.41, | 2-(3-chlorophenyl)-N′-(3,4- |
| | | | | | 359/361 | dimethoxybenylidine)cyclopropane- |
| | | | | | | carboxhydrazide |
| 34 | 2-F | — | OMe | OMe | 1.20, 343 | 2-(2-fluorophenyl)-N′-(3,4- |
| | | | | | | dimethoxybenylidine)cyclopropane- |
| | | | | | | carboxhydrazide |
| 35 | 3-F | — | OMe | OMe | 1.21, 343 | 2-(3-fluorophenyl)-N′-(3,4- |
| | | | | | | dimethoxybenylidine)cyclopropane- |
| | | | | | | carboxhydrazide |
| 36 | 4-F | — | OMe | OMe | 1.19, 343 | 2-(4-fluorophenyl)-N′-(3,4- |
| | | | | | | dimethoxybenylidine)cyclopropane- |
| | | | | | | carboxhydrazide |
| 37 | 2-Cl | — | CF₃ | — | 2.26, | 2-(2-chlorophenyl)-N′-(3- |
| | | | | | 367/369 | trifluoromethylbenylidine)cyclopropane- |
| | | | | | | carboxhydrazide |
| 38 | 3-Cl | — | CF₃ | — | 2.45, | 2-(3-chlorophenyl)-N′-(3- |
| | | | | | 367/369 | trifluoromethylbenylidine)cyclopropane- |
| | | | | | | carboxhydrazide |
| 39 | 4-Cl | — | CF₃ | — | 2.45, | 2-(4-chlorophenyl)-N′-(3- |
| | | | | | 367/369 | trifluoromethylbenylidine)cyclopropane- |
| | | | | | | carboxhydrazide |
| 40 | 2-F | — | CF₃ | — | 1.96, 351 | 2-(2-fluorophenyl)-N′-(3- |
| | | | | | | trifluoromethylbenylidine)cyclopropane- |
| | | | | | | carboxhydrazide |
| 41 | 3-F | — | CF₃ | — | 1.97, 351 | 2-(3-fluorophenyl)-N′-(3- |
| | | | | | | trifluoromethylbenylidine)cyclopropane- |
| | | | | | | carboxhydrazide |
| 42 | 4-F | — | CF₃ | — | 1.93, 351 | 2-(4-fluorophenyl)-N′-(3- |
| | | | | | | trifluoromethylbenylidine)cyclopropane- |
| | | | | | | carboxhydrazide |
| 43 | 2-Cl | — | OMe | — | 1.65, | 2-(2-chlorophenyl)-N′-(3- |
| | | | | | 329/331 | methoxybenylidine)cyclopropane- |
| | | | | | | carboxhydrazide |
| 44 | 3-Cl | — | OMe | — | 1.79, | 2-(3-chlorophenyl)-N′-(3- |
| | | | | | 329/331 | methoxybenylidine)cyclopropane- |
| | | | | | | carboxhydrazide |
| 45 | 4-Cl | — | OMe | — | 1.79, | 2-(4-chlorophenyl)-N′-(3- |
| | | | | | 329/331 | methoxybenylidine)cyclopropane- |
| | | | | | | carboxhydrazide |
| 46 | 2-F | — | OMe | — | 1.47, 313 | 2-(2-fluorophenyl)-N′-(3- |
| | | | | | | methoxybenylidine)cyclopropane- |
| | | | | | | carboxhydrazide |
| 47 | 3-F | — | OMe | — | 1.49, 313 | 2-(3-fluorophenyl)-N′-(3- |
| | | | | | | methoxybenylidine)cyclopropane- |
| | | | | | | carboxhydrazide |
| 48 | 4-F | — | OMe | — | 1.46, 313 | 2-(4-fluorophenyl)-N′-(3- |
| | | | | | | methoxybenylidine)cyclopropane- |
| | | | | | | carboxhydrazide |
| 49 | 2-Cl | — | SMe | — | 2.02, | 2-(2-chlorophenyl)-N′-(3- |
| | | | | | 345/347 | methylthiobenylidine)cyclopropane- |
| | | | | | | carboxhydrazide |
| 50 | 3-Cl | — | SMe | — | 2.24, | 2-(3-chlorophenyl)-N′-(3- |
| | | | | | 345/347 | methylthiobenylidine)cyclopropane- |
| | | | | | | carboxhydrazide |
| 51 | 4-Cl | — | SMe | — | 2.21, | 2-(4-chlorophenyl)-N′-(3- |
| | | | | | 345/347 | methylthiobenylidine)cyclopropane- |
| | | | | | | carboxhydrazide |
| 52 | 2-F | — | SMe | — | 1.78, 329 | 2-(2-fluorophenyl)-N′-(3- |
| | | | | | | methylthiobenylidine)cyclopropane- |
| | | | | | | carboxhydrazide |
| 53 | 3-F | — | SMe | — | 1.79, 329 | 2-(3-fluorophenyl)-N′-(3- |
| | | | | | | methylthiobenylidine)cyclopropane- |
| | | | | | | carboxhydrazide |
| 54 | 4-F | — | SMe | — | 1.76, 329 | 2-(4-fluorophenyl)-N′-(3- |
| | | | | | | methylthiobenylidine)cyclopropane- |
| | | | | | | carboxhydrazide |
| 55 | 2-Cl | CF₃ | — | — | 2.41, | 2-(2-chlorophenyl)-N′-(2- |
| | | | | | 367/369 | trifluoromethylbenylidine)cyclopropane- |
| | | | | | | carboxhydrazide |
| 56 | 3-Cl | CF₃ | — | — | 2.66, | 2-(3-chlorophenyl)-N′-(2- |
| | | | | | 367/369 | trifluoromethylbenylidine)cyclopropane- |
| | | | | | | carboxhydrazide |
| 57 | 4-Cl | CF₃ | — | — | 2.67, | 2-(4-chlorophenyl)-N′-(2- |
| | | | | | 367/369 | trifluoromethylbenylidine)cyclopropane- |
| | | | | | | carboxhydrazide |
| 58 | 2-F | CF₃ | — | — | 2.09, 351 | 2-(2-fluorophenyl)-N′-(2- |
| | | | | | | trifluoromethylbenylidine)cyclopropane- |
| | | | | | | carboxhydrazide |
| 59 | 3-F | CF₃ | — | — | 2.12, 351 | 2-(3-fluorophenyl)-N′-(2- |
| | | | | | | trifluoromethylbenylidine)cyclopropane- |
| | | | | | | carboxhydrazide |
| 60 | 4-F | CF₃ | — | — | 2.07, 351 | 2-(4-fluorophenyl)-N′-(2- |
| | | | | | | trifluoromethylbenylidine)cyclopropane- |
| | | | | | | carboxhydrazide |
| 61 | 2-Cl | — | — | CF₃ | 2.29, | 2-(2-chlorophenyl)-N′-(4- |
| | | | | | 367/369 | trifluoromethylbenylidine)cyclopropane- |
| | | | | | | carboxhydrazide |
| 62 | 3-Cl | — | — | CF₃ | 2.50, | 2-(3-chlorophenyl)-N′-(4- |
| | | | | | 367/369 | trifluoromethylbenylidine)cyclopropane- |
| | | | | | | carboxhydrazide |
| 63 | 4-Cl | — | — | CF₃ | 2.50, | 2-(4-chlorophenyl)-N′-(4- |
| | | | | | 367/369 | trifluoromethylbenylidine)cyclopropane- |
| | | | | | | carboxhydrazide |
| 64 | 2-F | — | — | CF₃ | 2.00, 351 | 2-(2-fluorophenyl)-N′-(4- |
| | | | | | | trifluoromethylbenylidine)cyclopropane- |
| | | | | | | carboxhydrazide |
| 65 | 3-F | — | — | CF₃ | 2.02, 351 | 2-(3-fluorophenyl)-N′-(4- |
| | | | | | | trifluoromethylbenylidine)cyclopropane- |
| | | | | | | carboxhydrazide |
| 66 | 4-F | — | — | CF₃ | 1.97, 351 | 2-(4-fluorophenyl)-N′-(4- |
| | | | | | | trifluoromethylbenylidine)cyclopropane |
| | | | | | | carboxhydrazide |

Chemical names for Examples 23-66 can be converted to structures standard nomenclature rules or ChemDraw Ultra 10.0.

### Example 67

### N′-(3,4-dimethoxybenzylidene)-2-(4,8-dimethylquinolin-2-ylthio)acetohydrazide

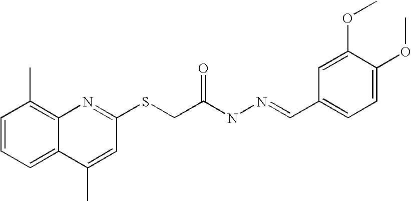

Molecular Formula: C₂₂H₂₃N₃O₃S; Molecular Weight (calc'd): 409.5.

### Example 68

### 3-(9H-Carbazol-9-yl)-N′-(3,4-dimethoxy-benzylidene)propanehydrazide

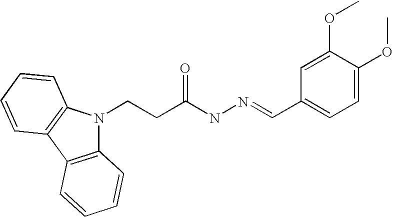

Molecular Formula: C₂₄H₂₃N₃O₃; Molecular Weight (calc'd): 401.5.

### Example 69

### Activity of Selected Compounds

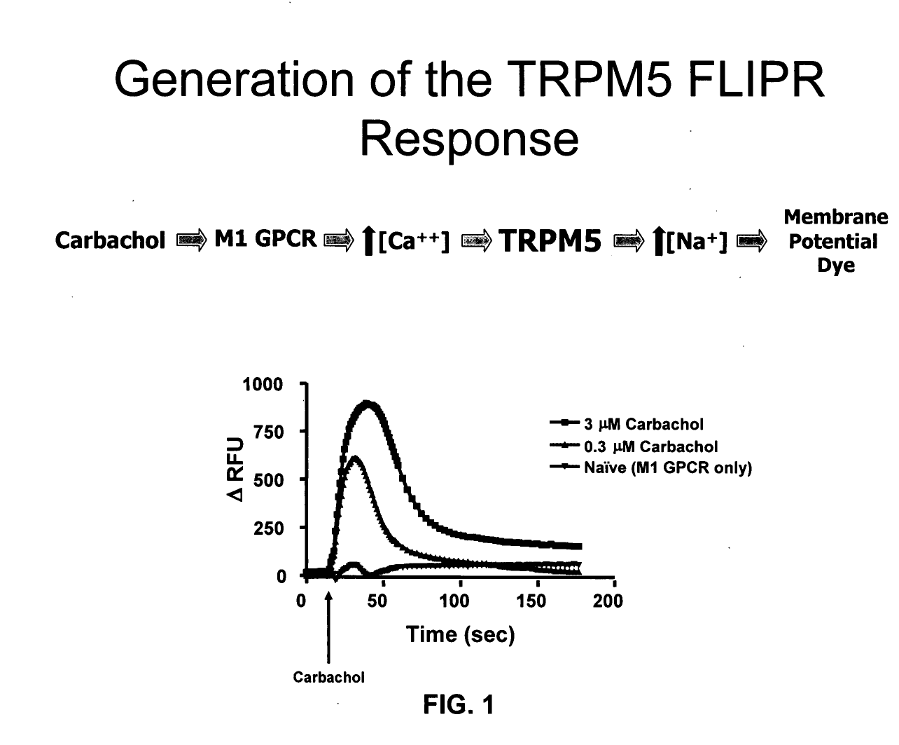

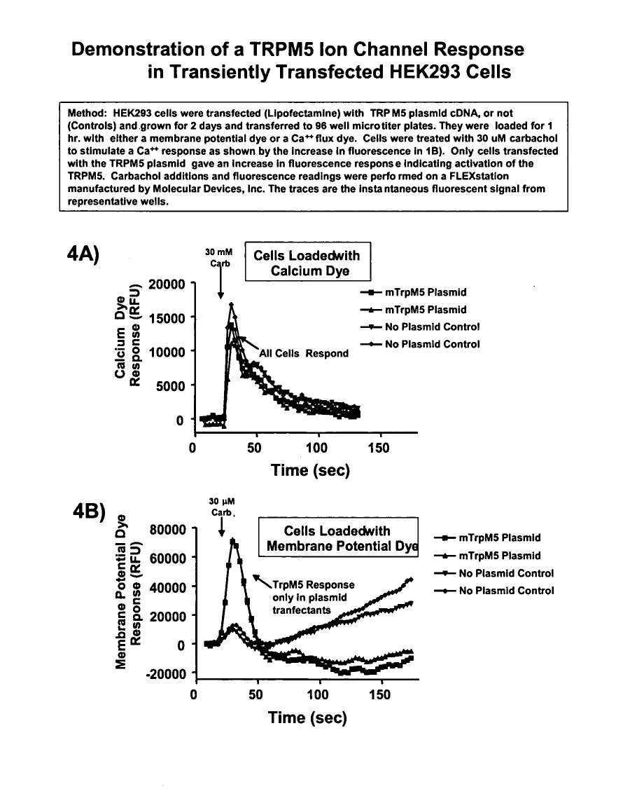

The activity of human TRPM5 ion channel was measured in live cells on a fluorescent imaging plate reader (FLIPR). The basis of the assay (shown in FIG. 1) is the calcium-dependent activation of the ion channel which occurs via by activation of a G-protein coupled receptor (GPCR). GPCR activation by an appropriate agonist causes a transient increase in intercellular Ca²⁺ ion concentration which in turn causes the ion channel to open, letting in Na⁺ ions. This influx causes a change in the membrane potential of the cell which can be monitored as a change in the fluorescent signal from voltage-dependent (membrane potential) fluorescent dyes. A demonstration of the assay is shown in FIGS. 4A and 4B, where traces of fluorescent response (Ex 530nm/Em565nm) versus time are shown for cells containing the plasmid and sham plasmid controls. While all cells gave a Ca²⁺ response to the endogenous muscarinic GPCR agonist carbachol (upper panel), only cells containing the plasmid showed a sharp peak for the membrane potential dye response (lower panel).

For the screening assay, the human TRPM5 gene was cloned, put into HEK293 cells, and a stable, high expression clone was used for screening. Cells were grown in standard media at 37° C. The day before screening, the cells were removed from flasks and added to 384 well clear bottom plates (8K cells in 20 μL/well). On the assay day, 20 μL of membrane potential dye (Part No. R8123, Molecular Devices Corp.) was added to the cells and dye was allowed to be taken up, i.e., load, into the cells for 1 hr at 37° C. The dye-loaded cell plate was placed in the FLIPR along with a second 384 well plate containing test compounds as well as positive (fully inhibited) and negative (non-inhibited) controls. The assay was started by addition of 10 μL of solution from the compound plate into the cell plate. During this process, continuous fluorescent recordings were made simultaneously for all wells. After addition of the compound solution, the tips were automatically washed and a stimulation solution of 3 μM ATP (an agonist for an endogenous purinurgic GPCR, was added to all wells of the cell plate. The height of the response was calculated and percent inhibition values, versus negative control wells, was calculated for the test samples.

Two counterscreen assays were run on separate cell plates utilizing the same cells as described above. In the calcium counterscreen, the cells were loaded with a calcium sensitive dye (Calcium3 Dye, Part no. 8090, Molecular Devices Corp.) and stimulated by ATP to check for compounds that block the GPCR-mediated calcium activation step. In the KCl counterscreen, cells are stimulated with 10 mM KCl instead of ATP to check for compounds that inhibit the membrane potential response by virtue of being non-specific ion channel blockers.

Unless otherwise indicated, the data in the table below were determined using the three assays described above, providing percent inhibition data at 10 μM.

| | | Calcium | KC1 |
|---|---|---|---|
| Example No. | TRPM5 Activity | Counterscreen | Counterscreen |
| 1 | 60 | −11 | 20 |
| 2 | 87 | −10 | 70 |
| 3 | 97 | 2 | 6 |
| 4 | 99 | −1 | −4 |
| 5 | 96 | −7 | 29 |
| 6 | 93 | 0 | −15 |
| 7 | 83 | −17 | 81 |
| 8 | 76 | −3 | 7 |
| 9 | 80 | 2 | 21 |
| 10 | 78 | −38 | −11 |
| 11 | 67 | 23 | 14 |
| 12 | 48 | −35 | −7 |
| 13 | 78 | 2 | 65 |
| 14 | 78 | −29 | 40 |
| 15 | 74 | 4 | 43 |
| 16 | 74 | −6 | −2 |
| 17 | 40 | −13 | 8 |
| 18 | 87 | −9 | 33 |
| 19 | 65 | 5 | 36 |
| 20 | 70 | −3 | 16 |
| 21 | 51 | 6 | 42 |
| 22 | 58 | −23 | 32 |

### Example 70

### Electrophysiological Results

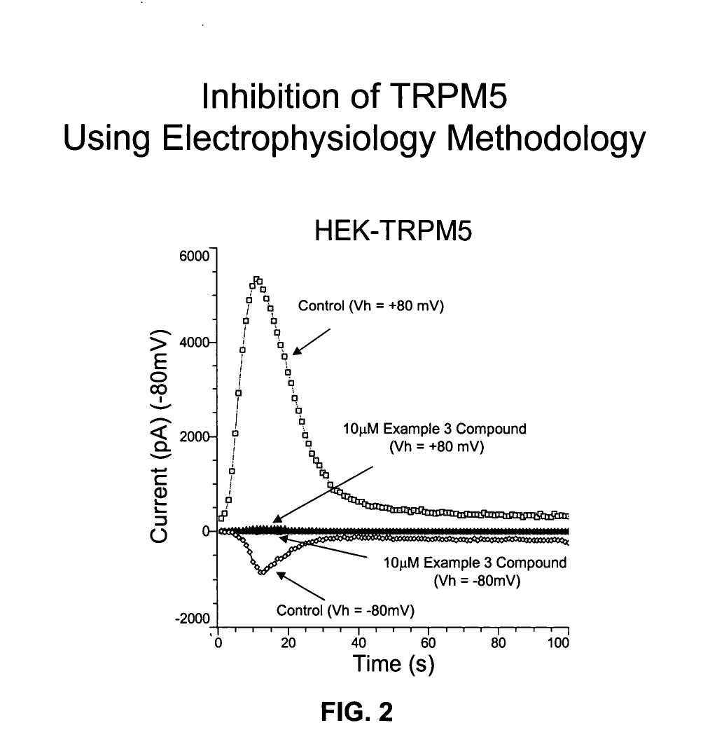

Standard whole-cell recordings were obtained from HEK cells stably transfected with human TRPM5. Internal solution contained 135 mM CsGlutamate, 10 mM HEPES, 2 mM MgATP, 5 mM CaCl₂ and 10 mM EGTA. External solution was HBSS (Gibco) buffered with 20 mM HEPES to pH 7.2. Currents were recorded with Multiclamp 700B amplifier using PClamp software; filered at 1 kHz, sampled at 5 kHz. Holding potential was −80 mV. TRPM5 current was activated by intracellular calcium dialysis (170 nM free calcium) and sampled with 200 ms ramps from −80 to 80 mV at 1 Hz. Current amplitudes were measured at −80 and 80 mV potted versus time. FIG. 2A shows a large >5 nA current (+80V) activated by calcium. Note that no significant current was seen in non-transfected, sham HEK cells (not shown) FIG. 2B shows >90% inhibition of TRPM5 current when TRPM5 transfected cells are pre-treated with 10 μM of Example 3.

Having now fully described this invention, it will be understood by those of ordinary skill in the art that the same can be performed within a wide and equivalent range of conditions, formulations and other parameters without affecting the scope of the invention or any embodiment thereof. All patents and publications cited herein are fully incorporated by reference herein in their entirety.

What is claimed is:

1. A method of inhibiting a taste, comprising administering to a subject in need of said taste inhibiting one or more compounds of Formula I: 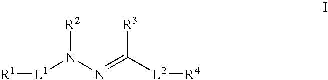
or a physiologically acceptable salt thereof, wherein
R¹ is C₆₋₁₄ aryl, 5-14 membered heteroaryl, C₃₋₁₄ cycloalkyl, C₃₋₁₄ cycloalkenyl, 3-14 membered cycloheteroalkyl, 3-14 membered cycloheteroalkenyl, and C-₁₋₆ alkyl, each of which is optionally substituted;
R² is H, C₁₋₆ alkyl, C₆₋₁₀ aryl, or C₆₋₁₀ aryl(C₁₋₆)alkyl;
R³ is H, C₁₋₆ alkyl, C₆₋₁₀ aryl, or cyano;
R⁴ is C₁₋₆ alkyl, C₆₋₁₄ aryl, 5-14 membered heteroaryl, C₃₋₁₄ cycloalkyl, C₃₋₁₄ cycloalkenyl, 3-14 membered cycloheteroalkyl, or 3-14 membered cycloheteroalkenyl, each of which is optionally substituted, or is cyano;
L¹ is absent, or is a linker containing 1-10 carbon and/or heteroatoms and which is optionally substituted;
L² is absent, or is a linker containing 1-10 carbon and/or heteroatoms and which is optionally substituted; or
R³, R⁴, and L², together with the carbon atom to which L²and R³ are attached, form a group selected from C₆₋₁₄ aryl, 5-14 membered heteroaryl, C₃₋₁₄ cycloalkyl, C₃₋₁₄ cycloalkenyl, 3-14 membered cycloheteroalkyl, 3-14 membered cycloheteroalkenyl, each of which is optionally substituted;
wherein said compound is administered in an amount sufficient to inhibit said taste.

2. The method according to claim 1, wherein R¹ is optionally substituted C₆₋₁₀ aryl.

3. The method according to claim 1, wherein R¹ is optionally substituted 5-14 membered heteroaryl.

4. The method according to claim 1, wherein R¹ is optionally substituted C₃₋₁₀ cycloalkyl or optionally substituted C₃₋₁₀ cycloalkenyl.

5. The method according to claim 1, wherein R¹ is optionally substituted 3-10 membered cycloheteroalkyl or optionally substituted 3-10 membered cycloheteroalkenyl.

6. The method according to claim 1, wherein R¹ is optionally substituted C₁₋₆ alkyl.

7. The method according to claim 1, wherein R² is H.

8. The method according to claim 1, wherein R² is C-₁₋₆ alkyl.

9. The method according to claim 1, wherein R² is C₆₋₁₀ aryl or C₆₋₁₀ aryl(C₁₋₆)alkyl.

10. The method according to claim 1, wherein R³ is H.

11. The method according to claim 1, wherein R³ is C₁₋₆ alkyl.

12. The method according to claim 1, wherein R³ is C₆₋₁₀ aryl.

13. The method according to claim 1, wherein R³ is cyano.

14. The method according to claim 1, wherein R⁴ is optionally substituted C₁₋₆ alkyl.

15. The method according to claim 1, wherein R⁴ is optionally substituted C₆₋₁₀ .

16. The method according to claim 1, wherein R⁴ is optionally substituted 5-10 membered heteroaryl.

17. The method according to claim 1, wherein R⁴ is optionally substituted C₃₋₁₀ cycloalkyl or optionally substituted C₃₋₁₀ cycloalkenyl.

18. The method according to claim 1, wherein R⁴ is optionally substituted 3-10 membered cycloheteroalkyl or optionally substituted 3-10 membered cycloheteroalkenyl.

19. The method according to claim 1, wherein L¹ is absent.

20. The method according to claim 1, wherein L¹ is a linker containing 1-10 carbon and/or heteroatoms and which is optionally substituted.

21. The method according to claim 1, wherein L¹ contains a cyclopropyl group.

22. The method according to claim 1, wherein L² is absent.

23. The method according to claim 1, wherein L² is a linker containing 1-10 carbon and/or heteroatoms and which is optionally substituted.

24. The method according to claim 1, wherein R¹ is unsubstituted phenyl.

25. The method according to claim 1, wherein R¹ is phenyl or naphthyl, each of which is substituted 1, 2, or 3 substituents independently selected from the group consisting of amino, hydroxy, nitro, halogen, cyano, thiol, C₁₋₆ alkyl, C₂₋₆ alkenyl, C₁₋₆ haloalkyl, C₁₋₆ alkoxy, C₃₋₆ alkenyloxy, C₁₋₆ alkylenedioxy, C₁₋₆ alkoxy(C₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, C₂₋₆ carboxyalkoxy, and C₂₋₆ carboxyalkyl.

26. The method according to claim 1, wherein R¹ is a nitrogen-containing heteroaryl.

27. The method according to claim 1, R¹ is selected from the group consisting of pyridyl, pyrimidinyl, imidazolyl, tetrazolyl, furanyl, thienyl, indolyl, azaindolyl, quinolinyl, pyrrolyl, benzimidazolyl, and benzothiazolyl, each of which is optionally substituted.

28. The method according to claim 1, wherein R⁴ is unsubstituted phenyl.

29. The method according to claim 1, wherein R⁴ is phenyl or naphthyl, each of which is substituted 1, 2, or 3 substituents independently selected from the group consisting of amino, hydroxy, nitro, halogen, cyano, thiol, C₁₋₆ alkyl, C₂₋₆ alkenyl, C₁₋₆ haloalkyl, C₁₋₆ alkoxy, C₃₋₆ alkenyloxy, C₁₋₆ alkylenedioxy, C₁₋₆ alkoxy(C₁₋₆)alkyl, C₁₋₆ aminoalkyl, C₁₋₆ aminoalkoxy, C₁₋₆ hydroxyalkyl, C₂₋₆ hydroxyalkoxy, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, carboxy, (C₁₋₆)alkoxy(C₂₋₆)alkoxy, C₂₋₆ carboxyalkoxy, and C₂₋₆ carboxyalkyl.

30. The method according to claim 1, wherein R⁴ is a nitrogen-containing heteroaryl.

31. The method according to claim 1, wherein R⁴ is selected from the group consisting of pyridyl, pyrimidinyl, imidazolyl, tetrazolyl, furanyl, thienyl, indolyl, azaindolyl, quinolinyl, pyrrolyl, benzimidazolyl, and benzothiazolyl, each of which is optionally substituted.

32. The method according to claim 1, wherein R¹ is optionally substituted C₆₋₁₀ aryl; R²is H or C₁₋₆ alkyl; R³ is H or C₁₋₆ alkyl; and R⁴ is optionally substituted C₆₋₁₀ aryl.

33. The method according to claim 1, wherein R¹is optionally substituted C₆₋₁₀ aryl; R² is H or C₁₋₆ alkyl; R³ is H or C₁₋₆ alkyl; and R⁴ is optionally substituted 5-10 membered heteroaryl.

34. The method according to claim 1, wherein R¹ is optionally substituted C₆₋₁₀ aryl; R² is H or C₁₋₆ alkyl; R³ is H or C₁₋₆ alkyl; and R⁴ is optionally substituted 5-10 membered heteroaryl.

35. The method according to claim 1, wherein R¹ is optionally substituted 5-10 membered heteroaryl; R² is H or C₁₋₆ alkyl; R³ is H or C₁₋₆ alkyl; and R⁴ is optionally substituted 5-10 membered heteroaryl.

36. The method according to claim 1, wherein R¹ is optionally substituted C₆₋₁₀ aryl; R² is H or C₁₋₆ alkyl; R³ is H or C₁₋₆ alkyl; and R⁴ is optionally substituted C₃₋₁₀ cycloalkyl.

37. The method according to claim 1, wherein R¹ is optionally substituted 5-10 membered heteroaryl; R² is H or C₁₋₆ alkyl; R³ is H or C₁₋₆ alkyl; and R⁴ and L² together form —N═N-aryl.

38. The method according to claim 1, wherein R¹ is optionally substituted 5-10 membered heteroaryl; R⁴ is optionally substituted C₆₋₁₀ aryl, such as phenyl and naphthyl; and L¹ and L² are absent.

39. The method according to claim 1, wherein R² is H, C₁₋₆ alkyl, or C₆₋₁₀ aryl(C₁₋₆)alkyl; L¹ is absent, or is a linker containing 1-6 carbon and/or heteroatoms and which is optionally substituted; R³, R⁴, and L² together with the carbon atom form a group selected from C₆₋₁₀ aryl, 5-10 membered heteroaryl, C₃₋₁₀ cycloalkyl, C₃₋₁₀ cycloalkenyl, 3-10 membered cycloheteroalkyl, 3-10 membered cycloheteroalkenyl, each of which is optionally substituted.

40. The method according to claim 1, wherein R¹ is heteroaryl; R² is H; R⁴ is heteroaryl; L¹ is absent; and L² is N═N.

41. The method according to claim 1, wherein R¹ is a bicycloalkyl; R² is H; R³ is H; R⁴ is aryl or heteroaryl; L¹ is absent; and L² is absent.

42. The method according to claim 1, wherein R¹ is aryl; R² is H; R³ is H; R⁴ is aryl or heteroaryl; L¹ is an optionally substituted a linker containing 2-4 carbon or hetero atoms; and L² is absent.

43. The method according to claim 1, wherein R¹ is cycloalkenyl; R² is H; R³ is H; R⁴ is aryl or heteroaryl; L¹ is an optionally substituted a linker containing 2-4 carbon or hetero atoms; and L² is absent.

44. The method according to claim 1, wherein the compound of Formula I selected from the group consisting of
methyl 4-((E)-((Z)-1-(2-(benzo[d]thiazol-2-1)hydrazono)-2-methyl-propyl)diazenyl)benzoate;
(E)-2-(4-bromo-2-((2-(quinolin-8-yl)hydrazono)methyl)phenoxy)acetic acid;
(E)-N′-(3,4-dimethoxybenzylidene)-2-(naphthalene-1-yl)acetohydrazide;
(E)-N′-(3,4-dimethoxybenzylidene)-2-phenylcyclopropane-carbohydrazide;
(E)-3-cyclohexenyl-4-hydroxy-N′-(4-methoxybenzylidene)-butanehydrazide;
(E)-N′-(3,4-dimethoxybenzylidene)-4-hydroxyhexanehydrazide;
2-((Z)-2-(phenyl-((E)-phenyldiazenyl)methylene)hydrazinyl)benzoic acid;
(E)-N′-(3,4-dimethoxybenzylidene)-2-(m-tolyloxy)acetohydrazide;
(E)-N′-(4-(allyloxy)-3-methoxybenzylidene)-2-(3-bromobenzylthio)-acetohydrazide;
(E)-N′-(4-isopropylbenzylidene)bicyclo[4.1.0]heptane-7-carbohydrazide;
(Z)-1,3,3-trimethyl-2-((E)-2-(2-(4-nitrophenyl)hydrazono)-ethylidene)indoline;
(E)-N′-(4-(diethylamino)-2-hydroxybenzylidene)-2-phenylcyclo-propanecarbohydrazide;
(4-(trifluoromethylthio)phenyl)carbonohydrazonoyldicyanide;
N-((E)-3-((Z)-2-(1,5-dimethyl-2-oxoindolin-3-ylidene)hydrazinyl)-3-oxo-1-phenylprop-1-en-2-yl)benzamide;
(Z)-2-(2-((1-butyl-1H-indol-3-yl)methylene)hydrazinyl)benzoic acid;
(E)-4-((2-benzyl-2-phenylhydrazono)methyl)pyridine;
(Z)-N′-((1H-Pyrrol-2-yl)methylene)tricyclo[3.3.1.1^{3,7}]decane-3-carbohydrazide;
(Z)-1-(2-(4-(ethyl(2-hydroxyethyl)amino)phenyl)hydrazono)-naphthalen-2-(1H)-one;
(E)-4-((2-(5-chloro-3-(trifluoromethyl)pyridini-2-yl)-2-2-methyl-hydrazono)methyl)benzene-1,3-diol;
(E)-2-(3,4-dimethylphenylamino)-N′-(4-morpholino-3-nitro-benzylidene)acetohydrazide;
(Z)-3-(2-nitro-5-(pyrrolidin-1-yl)phenyl)hydrazono)quinuclidine; and
(E)-2-((2-(1H-benzo[d]imidazol-2-yl)hydrazono)methyl)-5-(diethylamino)phenol.

45. The method according to claim 1, wherein the compound of Formula I is selected from the group consisting of
N-(3-(2-((6-Bromobenzo[d][1,3]dioxol-5-yl)methylene)hydrazinyl)-1-(4-(dimethylamino)phenyl)-3-oxoprop-1-en-2-yl)benzamide;
N-(1-(4-(Diethylamino)phenyl)-3-(2-(4-hydroxy-3-iodo-5-methoxybenzylidene)hydrazinyl)-3-oxoprop-1-en-2-yl)benzamide;
N′-(4-Hydroxy-3-methoxybenzylidene)-3-(1-hydroxycyclopentyl)-propanehydrazide;
4-Nitro-N′-(3,4,5-trimethoxybenzylidene)benzohydrazide;
N′-(4-(diethylamino)-2-hydroxybenylidine)phenylcyclopropane-carboxhydrazide;
N′-(5-Bromo-2-oxoindolin-3-ylidene)-2-(2-bromo-4-methoxyphenoxy)acetohydrazide;
3-(1H-indol-3-yl)-N′-(3,4,5-trimethoxybenzylidene)propanehydrazide;
N′-(2-oxoindolin-3-ylidene)-2-(2-methyl-4-(1,1-dimethylethyl)-phenoxy)acetohydrazide;
2-(4-Chlorophenyl)-N′-(3,4-dimethoxybenylidine)cyclopropane-carboxhydrazide;
2-(2-chlorophenyl)-N′-(3,4-dimethoxybenylidine)cyclopropane-carboxhydrazide;
2-(3-chlorophenyl)-N′-(3,4-dimethoxybenylidine)cyclopropane-carboxhydrazide;
2-(2-fluorophenyl)-N′-(3,4-dimethoxybenylidine)cyclopropane-carboxhydrazide;
2-(3-fluorophenyl)-N′-(3,4-dimethoxybenylidine)cyclopropane-carboxhydrazide;
2-(4-fluorophenyl)-N′-(3,4-dimethoxybenylidine)cyclopropane-carboxhydrazide;
2-(2-chlorophenyl)-N′-(3-trifluoromethylbenylidine)cyclopropane-carboxhydrazide;
2-(3-chlorophenyl)-N′-(3-trifluoromethylbenylidine)cyclopropane-carboxhydrazide;
2-(4-chlorophenyl)-N′-(3-trifluoromethylbenylidine)cyclopropane-carboxhydrazide;
2-(2-fluorophenyl)-N′-(3-trifluoromethylbenylidine)cyclopropane-carboxhydrazide;
2-(3-fluorophenyl)-N′-(3-trifluoromethylbenylidine)cyclopropane-carboxhydrazide;
2-(4-fluorophenyl)-N′-(3-trifluoromethylbenylidine)cyclopropane-carboxhydrazide;
2-(2-chlorophenyl)-N′-(3-methoxybenylidine)cyclopropane-carboxhydrazide;
2-(3-chlorophenyl)-N′-(3-methoxybenylidine)cyclopropane-carboxhydrazide;
2-(4-chlorophenyl)-N′-(3-methoxybenylidine)cyclopropane-carboxhydrazide;
2-(2-fluorophenyl)-N′-(3-methoxybenylidine)cyclopropane-carboxhydrazide;
2-(3-fluorophenyl)-N′-(3-methoxybenylidine)cyclopropane-carboxhydrazide;
2-(4-fluorophenyl)-N′-(3-methoxybenylidine)cyclopropane-carboxhydrazide;
2-(2-chlorophenyl)-N′-(3-methylthiobenylidine)cyclopropane-carboxhydrazide;
2-(3-chlorophenyl)-N′-(3-methylthiobenylidine)cyclopropane-carboxhydrazide;
2-(4-chlorophenyl)-N′-(3-methylthiobenylidine)cyclopropane-carboxhydrazide;
2-(2-fluorophenyl)-N′-(3-methylthiobenylidine)cyclopropane-carboxhydrazide;
2-(3-fluorophenyl)-N′-(3-methylthiobenylidine)cyclopropane-carboxhydrazide;
2-(4-fluorophenyl)-N′-(3-methylthiobenylidine)cyclopropane-carboxhydrazide;
2-(2-chlorophenyl)-N′-(2-trifluoromethylbenylidine)cyclopropane-carboxhydrazide;
2-(3-chlorophenyl)-N′-(2-trifluoromethylbenylidine)cyclopropane-carboxhydrazide;
2-(4-chlorophenyl)-N′-(2-trifluoromethylbenylidine)cyclopropane-carboxhydrazide;
2-(2-fluorophenyl)-N′-(2-trifluoromethylbenylidine)cyclopropane-carboxhydrazide;
2-(3-fluorophenyl)-N′-(2-trifluoromethylbenylidine)cyclopropane-carboxhydrazide;
2-(4-fluorophenyl)-N′-(2-trifluoromethylbenylidine)cyclopropane-carboxhydrazide;
2-(2-chlorophenyl)-N′-(4-trifluoromethylbenylidine)cyclopropane-carboxhydrazide;
2-(3-chlorophenyl)-N′-(4-trifluoromethylbenylidine)cyclopropane-carboxhydrazide;
2-(4-chlorophenyl)-N′-(4-trifluoromethylbenylidine)cyclopropane-carboxhydrazide;
2-(2-fluorophenyl)-N′-(4-trifluoromethylbenylidine)cyclopropane-carboxhydrazide;
2-(3-fluorophenyl)-N′-(4-trifluoromethylbenylidine)cyclopropane-carboxhydrazide;
2-(4-fluorophenyl)-N′-(4-trifluoromethylbenylidine)cyclopropane-carboxhydrazide;
N′-(3,4-dimethoxybenzylidene)-2-(4,8-dimethylquinolin-2-ylthio)-acetohydrazide;
3-(9H-carbazol-9-yl)-N′-(3,4-dimethoxybenzylidene)propane-hydrazide;
and physiologically acceptable salts thereof.

46. The method according to claim 1, wherein said subject is human.

47. The method according to claim 1, wherein the compound is administered in an amount from about 0.01 mg to about 100 mg.

48. The method according to claim 1, wherein the compound is administered as component of a pharmaceutical product.

49. The method according to claim 48, wherein the compound is present in the pharmaceutical product in an amount from about 0.01% to 50% by weight.

50. The method according to claim 1, wherein the compound is administered as component of a food product.

51. The method according to claim 50, wherein the compound is present in the food product in an amount from about 0.01% to 10% by weight.

52. The method according to claim 1, wherein the compound is administered as component of a dental hygienic product.

53. The method according to claim 52, wherein the compound is present in the dental hygienic product in an amount from about 0.01% to 20% by weight.

54. The method according to claim 1, wherein the taste is produced by a biologically active agent.

55. The method according to claim 1, wherein the taste is produced by one or more agents selected from the group consisting of antipyretics, analgesics, laxatives, appetite depressants, antacidics, antiasthmatics, antidiuretics, agents active against flatulence, antimigraine agents, psychopharmacological agents, spasmolytics, sedatives, antihyperkinetics, tranquilizers, antihistaminics, decongestants, beta-receptor blockers, agents for alcohol withdrawal, antitussives, fluorine supplements, local antibiotics, corticosteroid supplements, agents against goiter formation, antiepileptics, agents against dehydration, antiseptics, NSAIDs, gastrointestinal active agents, alkaloids, supplements for trace elements, ion-exchange resins, cholesterol-depressant agents, lipid-lowering agents, antiarrhythmics, and expectorants.

56. The method according to claim 1, wherein the taste is a bitter taste.

57. A method of inhibiting the depolarization of a taste receptor cell, comprising contacting said taste receptor cell with one or more compounds of Formula I: 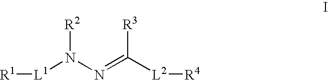
or a physiologically acceptable salt thereof, wherein
R¹ is C₆₋₁₄ aryl, 5-14 membered heteroaryl, C₃₋₁₄ cycloalkyl, C₃₋₁₄ cycloalkenyl, 3-14 membered cycloheteroalkyl, 3-14 membered cycloheteroalkenyl, and C₁₋₆ alkyl, each of which is optionally substituted;
R² is H, C₁₋₆ alkyl, C₆₋₁₀ aryl, or C₆₋₁₀ aryl(C₁₋₆)alkyl;
R³ is H, C₁₋₆ alkyl, C₆₋₁₀ aryl, or cyano;
R⁴ is C₁₋₆ alkyl, C₆₋₁₄ aryl, 5-14 membered heteroaryl, C₃₋₁₄ cycloalkyl, C₃₋₁₄ cycloalkenyl, 3-14 membered cycloheteroalkyl, or 3-14 membered cycloheteroalkenyl, each of which is optionally substituted, or is cyano;
L¹ is absent, or is a linker containing 1-10 carbon and/or heteroatoms and which is optionally substituted;
L² is absent, or is a linker containing 1-10 carbon and/or heteroatoms and which is optionally substituted; or
R³, R⁴, and L², together with the carbon atom to which L² and R³ are attached, form a group selected from C₆₋₁₄ aryl, 5-14 membered heteroaryl, C₃₋₁₄ cycloalkyl, C₃₋₁₄ cycloalkenyl, 3-14 membered cycloheteroalkyl, 3-14 membered cycloheteroalkenyl, each of which is optionally substituted;
wherein said compound is administered in an amount sufficient to inhibit the depolarization of a taste receptor cell.

58. A pharmaceutical composition comprising one or more pharmaceutically acceptable carriers and one or more compounds according to Formula I: 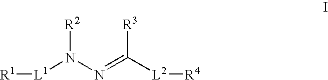
or physiologically acceptable salt thereof wherein
R¹ is C₆₋₁₄ aryl, 5-14 membered heteroaryl, C₃₋₁₄ cycloalkyl, C₃₋₁₄ cycloalkenyl, 3-14 membered cycloheteroalkyl, 3-14 membered cycloheteroalkenyl, and C₁₋₆ alkyl, each of which is optionally substituted;
R² is H, C₁₋₆ alkyl, C₆₋₁₀ aryl, or C₆₋₁₀ aryl(C₁₋₆)alkyl;
R³ is H, C₁₋₆ alkyl, C₆₋₁₀ aryl, or cyano;
R⁴ is C₁₋₆ alkyl, C₆₋₁₄ aryl, 5-14 membered heteroaryl, C₃₋₁₄ cycloalkyl, C₃₋₁₄ cycloalkenyl, 3-14 membered cycloheteroalkyl, or 3-14 membered cycloheteroalkenyl, each of which is optionally substituted, or is cyano;
L¹ is absent, or is a linker containing 1-10 carbon and/or heteroatoms and which is optionally substituted;
L² is absent, or is a linker containing 1-10 carbon and/or heteroatoms and which is optionally substituted; or
R³, R⁴, and L², together with the carbon atom to which L² and R³ are attached, form a group selected from C₆₋₁₄ aryl, 5-14 membered heteroaryl, C₃₋₁₄ cycloalkyl, C₃₋₁₄ cycloalkenyl, 3-14 membered cycloheteroalkyl, 3-14 membered cycloheteroalkenyl, each of which is optionally substituted.

59. A method of preparing an improved pharmaceutical composition, wherein the improvement comprises adding to a pharmaceutical composition one or more compounds according to Formula I: 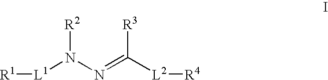
or physiologically acceptable salt thereof wherein
R¹ is C₆₋₁₄ aryl, 5-14 membered heteroaryl, C₃₋₁₄ cycloalkyl, C₃₋₁₄ cycloalkenyl, 3-14 membered cycloheteroalkyl, 3-14 membered cycloheteroalkenyl, and C₁₋₆ alkyl, each of which is optionally substituted;
R² is H, C₁₋₆ alkyl, C₆₋₁₀ aryl, or C₆₋₁₀ aryl(C₁₋₆)alkyl;
R³ is H, C₁₋₆ alkyl, C₆₋₁₀ aryl, or cyano;
R⁴ is C₁₋₆ alkyl, C₆₋₁₄ aryl, 5-14 membered heteroaryl, C₃₋₁₄ cycloalkyl, C₃₋₁₄ cycloalkenyl, 3-14 membered cycloheteroalkyl, or 3-14 membered cycloheteroalkenyl, each of which is optionally substituted, or is cyano;
L¹ is absent, or is a linker containing 1-10 carbon and/or heteroatoms and which is optionally substituted;
L² is absent, or is a linker containing 1-10 carbon and/or heteroatoms and which is optionally substituted; or
R³, R⁴, and L², together with the carbon atom to which L and R³ are attached, form a group selected from C₆₋₁₄ aryl, 5-14 membered heteroaryl, C₃₋₁₄ cycloalkyl, C₃₋₁₄ cycloalkenyl, 3-14 membered cycloheteroalkyl, 3-14 membered cycloheteroalkenyl, each of which is optionally substituted.

60. A food product comprising one or more food ingredients and one or more compounds according to Formula I: 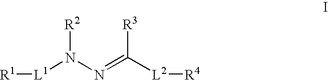
or physiologically acceptable salt thereof wherein
R¹ is C₆₋₁₄ aryl, 5-14 membered heteroaryl, C₃₋₁₄ cycloalkyl, C₃₋₁₄ cycloalkenyl, 3-14 membered cycloheteroalkyl, 3-14 membered cycloheteroalkenyl, and C₁₋₆ alkyl, each of which is optionally substituted;
R² is H, C₁₋₆ alkyl, C₆₋₁₀ aryl, or C₆₋₁₀ aryl(C₁₋₆)alkyl;
R³ is H, C₁₋₆ alkyl, C₆₋₁₀ aryl, or cyano;
R⁴ is C₁₋₆ alkyl, C₆₋₁₄ aryl, 5-14 membered heteroaryl, C₃₋₁₄ cycloalkyl, C₃₋₁₄ cycloalkenyl, 3-14 membered cycloheteroalkyl, or 3-14 membered cycloheteroalkenyl, each of which is optionally substituted, or is cyano;
L¹ is absent, or is a linker containing 1-10 carbon and/or heteroatoms and which is optionally substituted;
L² is absent, or is a linker containing 1-10 carbon and/or heteroatoms and which is optionally substituted; or
R³, R⁴, and L², together with the carbon atom to which L² and R³ are attached, form a group selected from C₆₋₁₄ aryl, 5-14 membered heteroaryl, C₃₋₁₄ cycloalkyl, C₃₋₁₄ cycloalkenyl, 3-14 membered cycloheteroalkyl, 3-14 membered cycloheteroalkenyl, each of which is optionally substituted.

61. A cosmetic product comprising one or more cosmetic ingredients and a compound according to Formula I: 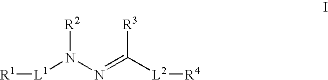
or physiologically acceptable salt thereof wherein
R¹ is C₆₋₁₄ aryl, 5-14 membered heteroaryl, C₃₋₁₄ cycloalkyl, C₃₋₁₄ cycloalkenyl, 3-14 membered cycloheteroalkyl, 3-14 membered cycloheteroalkenyl, and C₁₋₆ alkyl, each of which is optionally substituted;
R² is H, C₁₋₆ alkyl, C₆₋₁₀ aryl, or C₆₋₁₀ aryl(C₁₋₆)alkyl;
R³ is H, C₁₋₆ alkyl, C₆₋₁₀ aryl, or cyano;
R⁴ is C-₁₋₆ alkyl, C₆₋₁₄ aryl, 5-14 membered heteroaryl, C₃₋₁₄ cycloalkyl, C₃₋₁₄ cycloalkenyl, 3-14 membered cycloheteroalkyl, or 3-14 membered cycloheteroalkenyl, each of which is optionally substituted, or is cyano;
L¹ is absent, or is a linker containing 1-10 carbon and/or heteroatoms and which is optionally substituted;
L² is absent, or is a linker containing 1-10 carbon and/or heteroatoms and which is optionally substituted; or
R³, R⁴, and L², together with the carbon atom to which L² and R³ are attached, form a group selected from C₆₋₁₄ aryl, 5-14 membered heteroaryl, C₃₋₁₄ cycloalkyl, C₃₋₁₄ cycloalkenyl, 3-14 membered cycloheteroalkyl, 3-14 membered cycloheteroalkenyl, each of which is optionally substituted.

62. A method of preparing an improved cosmetic product, wherein the improvement comprises adding to a cosmetic product a compound according to Formula I: 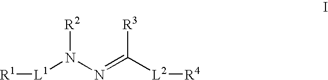
or physiologically acceptable salt thereof wherein
R¹ is C₆₋₁₄ aryl, 5-14 membered heteroaryl, C₃₋₁₄ cycloalkyl, C₃₋₁₄ cycloalkenyl, 3-14 membered cycloheteroalkyl, 3-14 membered cycloheteroalkenyl, and C₁₋₆ alkyl, each of which is optionally substituted;
R² is H, C₁₋₆ alkyl, C₆₋₁₀ aryl, or C₆₋₁₀ aryl(C₁₋₆)alkyl;
R³ is H, C₁₋₆ alkyl, C₆₋₁₀ aryl, or cyano;
R⁴ is C₁₋₆ alkyl, C₆₋₁₄ aryl, 5-14 membered heteroaryl, C₃₋₁₄ cycloalkyl, C₃₋₁₄ cycloalkenyl, 3-14 membered cycloheteroalkyl, or 3-14 membered cycloheteroalkenyl, each of which is optionally substituted, or is cyano;
L¹ is absent, or is a linker containing 1-10 carbon and/or heteroatoms and which is optionally substituted;
L² is absent, or is a linker containing 1-10 carbon and/or heteroatoms and which is optionally substituted; or
R³, R⁴, and L², together with the carbon atom to which L² and R³ are attached, form a group selected from C₆₋₁₄ aryl, 5-14 membered heteroaryl, C₃₋₁₄ cycloalkyl, C₃₋₁₄ cycloalkenyl, 3-14 membered cycloheteroalkyl, 3-14 membered cycloheteroalkenyl, each of which is optionally substituted.

63. A dental hygienic product comprising one or more dental hygienic ingredients and a compound according to Formula I: 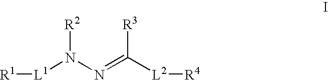
or physiologically acceptable salt thereof wherein
R¹ is C₆₋₁₄ aryl, 5-14 membered heteroaryl, C₃₋₁₄ cycloalkyl, C₃₋₁₄ cycloalkenyl, 3-14 membered cycloheteroalkyl, 3-14 membered cycloheteroalkenyl, and C₁₋₆ alkyl, each of which is optionally substituted;
R² is H, C₁₋₆ alkyl, C₆₋₁₀ aryl, or C₆₋₁₀ aryl(C₁₋₆)alkyl;
R³ is H, C₁₋₆ alkyl, C₆₋₁₀ aryl, or cyano;
R⁴ is C₁₋₆ alkyl, C₆₋₁₄ aryl, 5-14 membered heteroaryl, C₃₋₁₄ cycloalkyl, C₃₋₁₄ cycloalkenyl, 3-14 membered cycloheteroalkyl, or 3-14 membered cycloheteroalkenyl, each of which is optionally substituted, or is cyano;
L¹ is absent, or is a linker containing 1-10 carbon and/or heteroatoms and which is optionally substituted;
L² is absent, or is a linker containing 1-10 carbon and/or heteroatoms and which is optionally substituted; or
R³, R⁴, and L², together with the carbon atom to which L² and R³ are attached, form a group selected from C₆₋₁₄ aryl, 5-14 membered heteroaryl, C₃₋₁₄ cycloalkyl, C₃₋₁₄ cycloalkenyl, 3-14 membered cycloheteroalkyl, 3-14 membered cycloheteroalkenyl, each of which is optionally substituted.
